SEQUENCE LISTING (118 sequences)

SEQ ID NO: 1 - protein, 439 aa, synthetic construct
QVQLQQSGAELVKPGASVKLSCKASGYTFTSYDINWVRQRPEQGLEWIGWIFPGDDTTNYNENFKGKATLTADKSSSTAYMQLTRLTSEDSAVFFCARSVGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 19 aa, synthetic construct
MGWSCVFLFLLSVTAGVHS

SEQ ID NO: 3 - protein, 115 aa, synthetic construct
QVQLQQSGAELVKPGASVKLSCKASGYTFTSYDINWVRQRPEQGLEWIGWIFPGDDTTNYNENFKGKATLTADKSSSTAYMQLTRLTSEDSAVFFCARSVGFDYWGQGTTLTVSS

SEQ ID NO: 4 - protein, 5 aa, synthetic construct
SYDIN

SEQ ID NO: 5 - protein, 17 aa, synthetic construct
WIFPGDDTTNYNENFKG

SEQ ID NO: 6 - protein, 6 aa, synthetic construct
SVGFDY

SEQ ID NO: 7 - protein, 30 aa, synthetic construct
QVQLQQSGAELVKPGASVKLSCKASGYTFT

SEQ ID NO: 8 - protein, 14 aa, synthetic construct
WVRQRPEQGLEWIG

SEQ ID NO: 9 - protein, 32 aa, synthetic construct
KATLTADKSSSTAYMQLTRLTSEDSAVFFCAR

SEQ ID NO: 10 - protein, 11 aa, synthetic construct
WGQGTTLTVSS

SEQ ID NO: 11 - protein, 212 aa, synthetic construct
QIVLIQSPAIMSASPGEKVTMTCSASSSISYMYWYQQKPGTSPKRWIFETSKLASGVPARFSGSGSGTSYSLTISSMEAEDAATYYCHQRSSYPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 22 aa, synthetic construct
MDFQVRIFSFLLISASVILSRG

SEQ ID NO: 13 - protein, 105 aa, synthetic construct
QIVLIQSPAIMSASPGEKVTMTCSASSSISYMYWYQQKPGTSPKRWIFETSKLASGVPARFSGSGSGTSYSLTISSMEAEDAATYYCHQRSSYPTFGGGTKLEIK

SEQ ID NO: 14 - protein, 10 aa, synthetic construct
SASSSISYMY

SEQ ID NO: 15 - protein, 7 aa, synthetic construct
ETSKLAS

SEQ ID NO: 16 - protein, 8 aa, synthetic construct
HQRSSYPT

SEQ ID NO: 17 - protein, 23 aa, synthetic construct
QIVLIQSPAIMSASPGEKVTMTC

SEQ ID NO: 18 - protein, 15 aa, synthetic construct
WYQQKPGTSPKRWIF

SEQ ID NO: 19 - protein, 32 aa, synthetic construct
GVPARFSGSGSGTSYSLTISSMEAEDAATYYC

SEQ ID NO: 20 - protein, 10 aa, synthetic construct
FGGGTKLEIK

SEQ ID NO: 21 - DNA, 1317 bp, synthetic construct
  source 1..1317: mol_type = other DNA
caggttcagctgcagcagtctggagctgaactggtaaagcctggggcttcagtgaagttgtcctgcaaggcttctggctacaccttcacaagttatgatataaactgggtgaggcagaggcctgaacagggacttgagtggattggatggatttttcctggagatgatactactaactacaatgagaatttcaagggcaaggccacactgactgcagacaaatcctccagcacagcctacatgcagctcaccaggctgacatctgaggactctgctgtctttttctgtgcaagatcggtgggctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 22 - DNA, 57 bp, synthetic construct
  source 1..57: mol_type = other DNA
atgggatggagctgtgtctttctcttcctcctgtcagtaactgcaggtgtccactcc

SEQ ID NO: 23 - DNA, 345 bp, synthetic construct
  source 1..345: mol_type = other DNA
caggttcagctgcagcagtctggagctgaactggtaaagcctggggcttcagtgaagttgtcctgcaaggcttctggctacaccttcacaagttatgatataaactgggtgaggcagaggcctgaacagggacttgagtggattggatggatttttcctggagatgatactactaactacaatgagaatttcaagggcaaggccacactgactgcagacaaatcctccagcacagcctacatgcagctcaccaggctgacatctgaggactctgctgtctttttctgtgcaagatcggtgggctttgactactggggccaaggcaccactctcacagtctcctca

SEQ ID NO: 24 - DNA, 636 bp, synthetic construct
  source 1..636: mol_type = other DNA
caaattgttctcatccagtctccagcaatcatgtctgcatctccaggggagaaggtcaccatgacctgcagtgccagttcaagtataagttacatgtactggtaccagcagaagccaggcacctcccccaaaagatggatttttgaaacatccaaactggcttctggagtccctgctcgcttcagtggcagtgggtctgggacctcttattctctcacaatcagcagcatggaggctgaagatgctgccacttattactgccatcagcggagtagttaccccacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 25 - DNA, 66 bp, synthetic construct
  source 1..66: mol_type = other DNA
atggattttcaggtgcggattttcagcttcctgctaatcagtgcctcagtcatactgtccagagga

SEQ ID NO: 26 - DNA, 315 bp, synthetic construct
  source 1..315: mol_type = other DNA
caaattgttctcatccagtctccagcaatcatgtctgcatctccaggggagaaggtcaccatgacctgcagtgccagttcaagtataagttacatgtactggtaccagcagaagccaggcacctcccccaaaagatggatttttgaaacatccaaactggcttctggagtccctgctcgcttcagtggcagtgggtctgggacctcttattctctcacaatcagcagcatggaggctgaagatgctgccacttattactgccatcagcggagtagttaccccacgttcggaggggggaccaagctggaaataaaa

SEQ ID NO: 27 - protein, 5 aa, synthetic construct
DKTHT

SEQ ID NO: 28 - protein, 4 aa, synthetic construct
CPPC

SEQ ID NO: 29 - protein, 15 aa, synthetic construct
CPEPKSCDTPPPCPR

SEQ ID NO: 30 - protein, 12 aa, synthetic construct
ELKTPLGDTTHT

SEQ ID NO: 31 - protein, 10 aa, synthetic construct
KSCDKTHTCP

SEQ ID NO: 32 - protein, 7 aa, synthetic construct
KCCVDCP

SEQ ID NO: 33 - protein, 7 aa, synthetic construct
KYGPPCP

SEQ ID NO: 34 - protein, 15 aa, synthetic construct
EPKSCDKTHTCPPCP

SEQ ID NO: 35 - protein, 17 aa, synthetic construct
ELKTPLGDTTHTCPRCP

SEQ ID NO: 36 - protein, 12 aa, synthetic construct
SPNMVPHAHHAQ

SEQ ID NO: 37 - protein, 12 aa, synthetic construct
ESKYGPPCPPCP

SEQ ID NO: 38 - protein, 15 aa, synthetic construct
EPKSCDKTYTCPPCP

SEQ ID NO: 39 - protein, 45 aa, synthetic construct
TTTPAPRPPTPAPTIASQPLSLRPEACRPAAGGAVHTRGLDFACD

SEQ ID NO: 40 - protein, 110 aa, synthetic construct
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 41 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 27 aa, synthetic construct
FWVLVVVGGVLACYSLLVTVAFIIFWV

SEQ ID NO: 43 - protein, 23 aa, synthetic construct
LGLLVAGVLVLLVSLGVAIHLCC

SEQ ID NO: 44 - protein, 25 aa, synthetic construct
ALIVLGGVAGLLLFIGLGIFFCVRC

SEQ ID NO: 45 - protein, 23 aa, synthetic construct
LCYLLDGILFIYGVILTALFLRV

SEQ ID NO: 46 - protein, 26 aa, synthetic construct
WVLVVVGGVLACYSLLVTVAFIIFWV

SEQ ID NO: 47 - protein, 26 aa, synthetic construct
VAAILGLGLVLGLLGPLAILLALYLL

SEQ ID NO: 48 - protein, 24 aa, synthetic construct
ALPAALAVISFLLGLGLGVACVLA

SEQ ID NO: 49 - protein, 113 aa, synthetic construct
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 50 - protein, 112 aa, synthetic construct
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 51 - protein, 102 aa, synthetic construct
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 52 - protein, 101 aa, synthetic construct
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 53 - protein, 86 aa, synthetic construct
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 54 - protein, 21 aa, synthetic construct
DGVYTGLSTRNQETYETLKHE

SEQ ID NO: 55 - protein, 171 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 56 - protein, 127 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 57 - protein, 21 aa, synthetic construct
DQVYQPLRDRDDAQYSHLGGN

SEQ ID NO: 58 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 59 - protein, 21 aa, synthetic construct
NPDYEPIRKGQRDLYSGLNQR

SEQ ID NO: 60 - protein, 182 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 61 - protein, 21 aa, synthetic construct
DQLYQPLKDREDDQYSHLQGN

SEQ ID NO: 62 - protein, 163 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 21 aa, synthetic construct
NQLYNELNLGRREEYDVLDKR

SEQ ID NO: 66 - protein, 22 aa, synthetic construct
EGLYNELQKDKMAEAYSEIGMK

SEQ ID NO: 67 - protein, 21 aa, synthetic construct
DGLYQGLSTATKDTYDALHMQ

SEQ ID NO: 68 - protein, 226 aa, synthetic construct
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 69 - protein, 188 aa, synthetic construct
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 70 - protein, 21 aa, synthetic construct
ENLYEGLNLDDCSMYEDISRG

SEQ ID NO: 71 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 72 - protein, 42 aa, synthetic construct
KRGRKKLLYIFKQPFMRPVQTTQEEDGCSCRFPEEEEGGCEL

SEQ ID NO: 73 - protein, 44 aa, synthetic construct
FWVRSKRSRLLHSDYMNMTPRRPGPTRKHYQPYAPPRDFAAYRS

SEQ ID NO: 74 - protein, 35 aa, synthetic construct
TKKKYSSSVHDPNGEYMFMRAVNTAKKSRLTDVTL

SEQ ID NO: 75 - protein, 37 aa, synthetic construct
RRDQRLPPDAHKPPGGGSFRTPIQEEQADAHSTLAKI

SEQ ID NO: 76 - protein, 114 aa, synthetic construct
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 77 - protein, 49 aa, synthetic construct
HQRRKYRSNKGESPVEPAEPCRYSCPREEEGSTIPIQEDYRKPEPACSP

SEQ ID NO: 78 - protein, 187 aa, synthetic construct
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 79 - protein, 54 aa, synthetic construct
HIWQLRSQCMWPRETQLLLEVPPSTEDARSCQFPEEERGERSAEEKGRLGDLWV

SEQ ID NO: 80 - protein, 60 aa, synthetic construct
CVKRRKPRGDVVKVIVSVQRKRQEAEGEATVIEALQAPPDVTTVAVEETIPSFTGRSPNH

SEQ ID NO: 81 - protein, 41 aa, synthetic construct
RSKRSRGGHSDYMNMTPRRPGPTRKHYQPYAPPRDFAAYRS

SEQ ID NO: 82 - protein, 9 aa, synthetic construct
YPYDVPDYA

SEQ ID NO: 83 - protein, 8 aa, synthetic construct
DYKDDDDK

SEQ ID NO: 84 - protein, 10 aa, synthetic construct
EQKLISEEDL

SEQ ID NO: 85 - protein, 4 aa, synthetic construct
GGSG

SEQ ID NO: 86 - protein, 5 aa, synthetic construct
GGSGG

SEQ ID NO: 87 - protein, 5 aa, synthetic construct
GSGSG

SEQ ID NO: 88 - protein, 5 aa, synthetic construct
GSGGG

SEQ ID NO: 89 - protein, 5 aa, synthetic construct
GGGSG

SEQ ID NO: 90 - protein, 5 aa, synthetic construct
GSSSG

SEQ ID NO: 91 - protein, 5 aa, synthetic construct
EAAAK

SEQ ID NO: 92 - protein, 10 aa, synthetic construct
EAAAKEAAAK

SEQ ID NO: 93 - protein, 15 aa, synthetic construct
EAAAKEAAAKEAAAK

SEQ ID NO: 94 - protein, 20 aa, synthetic construct
EAAAKEAAAKEAAAKEAAAK

SEQ ID NO: 95 - protein, 25 aa, synthetic construct
EAAAKEAAAKEAAAKEAAAKEAAAK

SEQ ID NO: 96 - protein, 30 aa, synthetic construct
EAAAKEAAAKEAAAKEAAAKEAAAKEAAAK

SEQ ID NO: 97 - protein, 35 aa, synthetic construct
EAAAKEAAAKEAAAKEAAAKEAAAKEAAAKEAAAK

SEQ ID NO: 98 - protein, 40 aa, synthetic construct
EAAAKEAAAKEAAAKEAAAKEAAAKEAAAKEAAAKEAAAK

SEQ ID NO: 99 - protein, 45 aa, synthetic construct
EAAAKEAAAKEAAAKEAAAKEAAAKEAAAKEAAAKEAAAKEAAAK

SEQ ID NO: 100 - protein, 50 aa, synthetic construct
EAAAKEAAAKEAAAKEAAAKEAAAKEAAAKEAAAKEAAAKEAAAKEAAAK

SEQ ID NO: 101 - protein, 25 aa, synthetic construct
LVGTDATLCCSFSPEPGFSLAQLNL

SEQ ID NO: 102 - protein, 25 aa, synthetic construct
AQLNLIWQLTDTKQLVHSFAEGQDQ

SEQ ID NO: 103 - protein, 25 aa, synthetic construct
EGQDQGSAYANRTALFPDLLAQGNA

SEQ ID NO: 104 - protein, 25 aa, synthetic construct
AQGNASLRLQRVRVADEGSFTCFVS

SEQ ID NO: 105 - protein, 25 aa, synthetic construct
TCFVSIRDFGSAAVSLQVAAPYSKP

SEQ ID NO: 106 - protein, 25 aa, synthetic construct
PYSKPSMTLEPNKDLRPGDTVTITC

SEQ ID NO: 107 - protein, 25 aa, synthetic construct
VTITCSSYRGYPEAEVFWQDGQGVP

SEQ ID NO: 108 - protein, 25 aa, synthetic construct
GQGVPLTGNVTTSQMANEQGLFDVH

SEQ ID NO: 109 - protein, 25 aa, synthetic construct
LFDVHSVLRVVLGANGTYSCLVRNP

SEQ ID NO: 110 - protein, 25 aa, synthetic construct
LVRNPVLQQDAHGSVTITGQPMTFP

SEQ ID NO: 111 - DNA, 1389 bp, synthetic construct
  source 1..1389: mol_type = other DNA
atgcaactcctgtcttgcattgcactaagtcttgcacttgtcacgaattcgcaggttcagctgcagcagtctggagctgaactggtaaagcctggggcttcagtgaagttgtcctgcaaggcttctggctacaccttcacaagttatgatataaactgggtgaggcagaggcctgaacagggacttgagtggattggatggatttttcctggagatgatactactaactacaatgagaatttcaagggcaaggccacactgactgcagacaaatcctccagcacagcctacatgcagctcaccaggctgacatctgaggactctgctgtctttttctgtgcaagatcggtgggctttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 112 - DNA, 690 bp, synthetic construct
  source 1..690: mol_type = other DNA
atgcaactcctgtcttgcattgcactaagtcttgcacttgtcacgaattcacaaattgttctcatccagtctccagcaatcatgtctgcatctccaggggagaaggtcaccatgacctgcagtgccagttcaagtataagttacatgtactggtaccagcagaagccaggcacctcccccaaaagatggatttttgaaacatccaaactggcttctggagtccctgctcgcttcagtggcagtgggtctgggacctcttattctctcacaatcagcagcatggaggctgaagatgctgccacttattactgccatcagcggagtagttaccccacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 113 - protein, 462 aa, synthetic construct
MQLLSCIALSLALVTNSQVQLQQSGAELVKPGASVKLSCKASGYTFTSYDINWVRQRPEQGLEWIGWIFPGDDTTNYNENFKGKATLTADKSSSTAYMQLTRLTSEDSAVFFCARSVGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 229 aa, synthetic construct
MQLLSCIALSLALVTNSQIVLIQSPAIMSASPGEKVTMTCSASSSISYMYWYQQKPGTSPKRWIFETSKLASGVPARFSGSGSGTSYSLTISSMEAEDAATYYCHQRSSYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 5 aa, synthetic construct
GSGGS

SEQ ID NO: 116 - protein, 4 aa, synthetic construct
GGGS

SEQ ID NO: 117 - protein, 534 aa, synthetic construct
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 118 - DNA, 21 bp, synthetic construct
  source 1..21: mol_type = other DNA
aaaggccagtattaaggctag

(19) United States
(12) Patent Application Publication
(10) Pub. No.: US 2025/0230244 A1
(43) Pub. Date: Jul. 17, 2025

(54) METHODS AND COMPOSITIONS COMPRISING B7-H3 BINDING POLYPETIDES

(71) Applicants: BOARD OF REGENTS, THE UNIVERSITY OF TEXAS SYSTEM, Austin, TX (US); EBERHARD KARLS UNIVERSITAET TUEBINGEN MEDIZINISCHE FAKULTAET, Tuebingen (DE)

(72) Inventors: Venkata Lokesh BATTULA, Houston, TX (US); Hans-Jorg BUHRING, Houston, TX (US)

(73) Assignees: BOARD OF REGENTS, THE UNIVERSITY OF TEXAS SYSTEM, Austin, TX (US); EBERHARD KARLS UNIVERSITAET TUEBINGEN MEDIZINISCHE FAKULTAET, Tuebingen (DE)

(21) Appl. No.: 18/699,089

(22) PCT Filed: Oct. 5, 2022
(86) PCT No.: PCT/US2022/077623

§ 371 Date: Apr. 5, 2024

Related U.S. Application Data
(60) Provisional application No. 63/252,477, filed on Oct. 5, 2021.

(51) Int. Cl. C07K 16/28; A61K 31/513; A61K 35/12; A61P 35/02; C12N 5/0784; C12N 15/11

(52) U.S. Cl. CPC C07K 16/2827; A61K 31/513; A61K 35/12; A61P 35/02; C12N 5/0639; C12N 15/11; C07K 2317/24; C07K 2317/565

(57) ABSTRACT

The disclosure describes antibodies, antigen binding fragments, scFvs, CARs, and polypeptides that bind to B7H3. Further aspects relate to polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure.

This application claims priority of U.S. Provisional Patent Application No. 63/252,477 filed Oct. 5, 2021, which is hereby incorporated by reference in its entirety.

### BACKGROUND

### I. Field of the Invention

Aspects of the invention relate to at least the fields of cancer biology and molecular biology.

### II. Background

Acute myeloid leukemia (AML) is an aggressive malignancy characterized by a block in myeloid differentiation, leading to uncontrolled proliferation of myeloblasts in blood and bone marrow (1). AML is the most commonly reported leukemia in adults, with an incidence rate of 4.3 cases per 100,000 every year (2, 3). Despite recent advances in the development of targeted therapeutic approaches, AML remains an aggressive malignancy with very high relapse rates and is associated with poor overall survival (4). Evidently, there is a dire need to identify clinically relevant therapeutic targets in combination with standard chemotherapy for AML patients. One such approach that has recently gained momentum is cancer immunotherapy. Targeting immune checkpoint regulatory molecules in combination with standard chemotherapy has been shown to be more effective than single-agent chemotherapeutic regimens (5, 6).

B7 homolog 3 (B7-H3), an immune checkpoint molecule of the B7 family, is a co-receptor of a type I transmembrane protein (7). In humans, B7-H3 exists in two isoforms (2Ig-B7-H3 and 4Ig-B7-H3), whereas a single isoform (2Ig-B7-H3) has been reported in mice (7, 8). Although the receptor of B7-H3 is still not known, the FG loop region on B7-H3 is known to be responsible for maintaining the immunomodulatory function of B7-H3 (9). The exact role of the B7-H3 molecule in regulating the immune cell response is not distinctly defined. In view of the limited studies to date, it is essential to develop an in-depth understanding of B7-H3's immunomodulatory roles in AML progression and assess its therapeutic potential.

### SUMMARY

To address the need, the inventors, mAbs against B7-H3 have been generated. The disclosure describes methods for treating cancer in a subject comprising administering to the subject an anticancer treatment after a biological sample from the subject has been analyzed for B7-H3 expression. Also described is a method for prognosing a subject having cancer or for predicting a cancer subject's response to an anticancer treatment, the method comprising evaluating a biological sample from the subject for B7-H3 expression. Further aspects relate to a method comprising evaluating the expression level of B7-H3 in a biological sample from a subject having cancer. Aspects of the disclosure relate to an antibody or antigen binding fragment comprising a heavy chain variable region (VH) and a light chain variable region (VL), wherein the VH comprises a HCDR1, HCDR2, and HCDR3 from the VH of SEQ ID NO:3 and wherein the VL comprises a LCDR1, LCDR2, and LCDR3 from the VL of SEQ ID NO:13. In some aspects, the antibody or antigen binding fragment comprising a heavy chain variable region (VH) and a light chain variable region (VL), wherein the VH comprises a HCDR1, HCDR2, and HCDR3 that has at least 80% sequence identity with a HCDR1, HCDR2, and HCDR3 from the VH of SEQ ID NO:3 and wherein the VL comprises a LCDR1, LCDR2, and LCDR3 that has at least 80% sequence identity with a LCDR1, LCDR2, and LCDR3 from the VL of SEQ ID NO:13. Aspects also describe an antibody or antigen binding fragment comprising a VH and a VL, wherein the VH comprises a HCDR1, HCDR2, and HCDR3 comprising the amino acid sequence of SEQ ID NOS:4-6, respectively, and wherein the VL comprises a LCDR1, LCDR2, and LCDR3 comprising the amino acid sequence of SEQ ID NOS:14-16, respectively. Further aspects describe an antibody or antigen binding fragment comprising a VH and a VL, wherein the VH comprises a HCDR1, HCDR2, and HCDR3 having at least 80% sequence identity to SEQ ID NOS:4-6, respectively, and wherein the VL comprises a LCDR1, LCDR2, and LCDR3 having at least 80% sequence identity to SEQ ID NOS:14-16, respectively.

Aspects of the disclosure relate to a polypeptide comprising a single chain variable fragment (scFv) comprising a VH and a VL wherein the VH comprises a HCDR1, HCDR2, and HCDR3 from the VH of SEQ ID NO:3 and wherein the VL comprises a LCDR1, LCDR2, and LCDR3 from the VL of SEQ ID NO:13. In some aspects, the polypeptide comprises a scFv comprising a VH and a VL wherein the VH comprises a HCDR1, HCDR2, and HCDR3 that has at least 80% sequence identity with a HCDR1, HCDR2, and HCDR3 from the VH of SEQ ID NO:3 and wherein the VL comprises a LCDR1, LCDR2, and LCDR3 that has at least 80% sequence identity with a LCDR1, LCDR2, and LCDR3 from the VL of SEQ ID NO:13. Aspects also describe a polypeptide comprising a scFv comprising a VH and a VL, wherein the VH comprises a HCDR1, HCDR2, and HCDR3 comprising the amino acid sequence of SEQ ID NOS:4-6, respectively, and wherein the VL comprises a LCDR1, LCDR2, and LCDR3 comprising the amino acid sequence of SEQ ID NOS:14-16, respectively. Further aspects describe a polypeptide comprising a scFv comprising VH and a VL, wherein the VH comprises a HCDR1, HCDR2, and HCDR3 having at least 80% sequence identity to SEQ ID NOS:4-6, respectively, and wherein the VL comprises a LCDR1, LCDR2, and LCDR3 having at least 80% sequence identity to SEQ ID NOS:14-16, respectively. Aspects of the disclosure also describe a chimeric antigen receptor (CAR) comprising: a) an extracellular binding domain comprising a polypeptide of the disclosure; b) a transmembrane domain; and, c) a cytoplasmic region comprising a costimulatory domain and a primary intracellular signaling domain.

The disclosure also describes compositions comprising antibodies, antigen binding fragments, polypeptides, scFvs, or CARs of the disclosure. Further aspects relate to one or more nucleic acids encoding an antibody, antigen binding fragment, polypeptide, scFv, or CAR of the disclosure. Aspects relate to a nucleic acid encoding an antibody heavy chain or VH, wherein the nucleic acid has at least 70% sequence identity to SEQ ID NO:21 or 23. Further aspects relate to a nucleic acid encoding an antibody light chain or VL, wherein the nucleic acid has at least 70% sequence identity to SEQ ID NO:24 or 26. Also described are vectors, such as expression vectors comprising the nucleic acids of the disclosure. Further aspects relate to cells comprising the nucleic acids, polypeptides, antibodies, CARs, and/or scFvs of the disclosure. Further aspects relate to a method of a making a cell comprising transferring a nucleic acid(s) or vector of the disclosure into a cell. Yet further aspects relate to a method for making a polypeptide comprising transferring a nucleic acid(s) or vector of the disclosure into a cell under conditions sufficient to express polypeptides from the nucleic acids. The method may further comprise isolating the expressed polypeptides.

Further aspects relate to a method for treating or preventing cancer in a subject, the method comprising administering to the subject, an antibody, antigen binding fragment, polypeptide, composition, scFv, CAR, or cell of the disclosure. Yet further aspects relate to a method of stimulating an immune response in a subject, the method comprising administering to the subject, an antibody, antigen binding fragment, polypeptide, composition, scFv, CAR, or cell of the disclosure. Further aspects relate to a method for treating acute myelogenous leukemia in a subject, the method comprising administering to the subject an anti-B7-H3 antibody in combination with a BCL-2 inhibitor. In some aspects, the BCL-2 inhibitor comprises venetocolax. In some aspects, the anti-B7-H3 antibody comprises an antibody of the disclosure. Further aspects relate to a method for evaluating a sample from a subject, the method comprising contacting a biological sample from the subject, or extract thereof, with at least one antibody, antigen binding fragment, or polypeptide of the disclosure.

Further aspects of the disclosure relate to an isolated peptide comprising at least 70% sequence identity to a peptide of SEQ ID NO:105 or 109. In some aspects, the peptide comprises at least 6 contiguous amino acids of a peptide of SEQ ID NO:105 or 109. Further aspects relate to pharmaceutical compositions comprising the isolated peptide, nucleic acids encoding the peptide, and expression vectors and host cells comprising the nucleic acids of the disclosure. Also provided is an in vitro isolated dendritic cell comprising a peptide, nucleic acid, or expression vector of the disclosure.

Further aspects relate to a method of making a cell comprising transferring a nucleic acid or expression vector of the disclosure into a cell, such as a host cell. In some aspects, the method further comprises isolating the expressed peptide or polypeptide. Other aspects of the disclosure relate to a method of producing peptide-specific immune effector cells comprising: (a) obtaining a starting population of immune effector cells; and (b) contacting the starting population of immune effector cells with a peptide of the disclosure, thereby generating peptide-specific immune effector cells.

The disclosure also describes peptide-specific engineered T cells produced according to the methods of the disclosure and pharmaceutical compositions comprising the engineered T cells. Further aspects relate to a method of treating or preventing cancer in a subject, the method comprising administering an effective amount of a peptide, pharmaceutical composition, nucleic acid, dendritic cell, or peptide-specific T cell of the disclosure. Yet further aspects relate to a method of cloning a peptide-specific T cell receptor (TCR), the method comprising (a) obtaining a starting population of immune effector cells; (b) contacting the starting population of immune effector cells with the peptide of the disclosure, thereby generating peptide-specific immune effector cells; (c) purifying immune effector cells specific to the peptide, and (d) isolating a TCR sequence from the purified immune effector cells. Also provide is a method for prognosing a patient or for detecting T cell responses in a patient, the method comprising: contacting a biological sample from the patient with a peptide of the disclosure. Further aspects relate to kits comprising a peptide, nucleic acid, expression vector, or composition of the disclosure.

The anticancer treatment in methods of the disclosure may comprise a B7-H3 targeting agent. The B7-H3 targeting agent may comprise an antibody, a polypeptide, an antigen binding fragment of an antibody, a single chain variable fragment (scFv) or a chimeric antigen receptor (CAR). In some aspects, the B7-H3 targeting agent comprises a cell comprising a nucleic acid encoding an anti-B7H3 scFv, polypeptide, or CAR. The biological sample from the subject may be one that has been determined to have high expression of Bf7-H3. In some aspects, the biological sample has been determined to have high high expression of B7-H3 relative to a control, wherein the control is a cut-off value or wherein the control is level of B7-H3 expression in a biological sample from a subject or the average level of B7-H3 expression in biological samples from subjects determined to not have cancer. In some aspects, the biological sample comprises a tissue sample, a cancerous sample, a tumor sample, or a sample obtained from a biopsy. The biological sample may be one that has been evaluated as having high B7-H3 expression in some aspects. Alternatively, the biological sample may be one that has been evaluated as having low B7-H3 expression. The subject may be prognosed as high risk for unfavorable clinical outcomes in the methods of the disclosure. For example, the subject may be prognosed as likely to have unfavorable clinical outcomes when high expression of B7-H3 is detected in a biological sample from the subject. In some aspects, the subject is predicted to respond an anticancer treatment comprising a B7-H3 targeting therapy. For example, a subject may be predicted to respond to a B7-H3 targeting therapy when high expression of B7-H3 is detected in the biological sample from the subject. The subject may be prognosed as having favorable clinical outcomes, such as when low expression of B7-H3 is detected in the biological sample from the subject. In certain aspects, the subject is treated with an anticancer treatment and wherein the anticancer treatment excludes a B7-H3 targeting agent. The clinical outcomes may comprise one or more of overall survival, disease-free survival, progression-free survival, time-to-progression, and objective response rate.

In some aspects, the HCDR1 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with the HCDR1 from the VH of SEQ ID NO:3. In some aspects, the HCDR2 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with the HCDR2 from the VH of SEQ ID NO:3. In some aspects, the HCDR3 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with the HCDR3 from the VH of SEQ ID NO:3. In some aspects, the LCDR1 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with the LCDR1 from the VL of SEQ ID NO:13. In some aspects, the LCDR2 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with the LCDR2 from the VL of SEQ ID NO:13. In some aspects, the LCDR3 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with the LCDR3 from the VL of SEQ ID NO:13.

In some aspects, the HCDR1 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:4. In some aspects, the HCDR2 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:5. In some aspects, the HCDR3 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:6. In some aspects, the LCDR1 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:14. In some aspects, the LCDR2 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:15. In some aspects, the LCDR3 comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:16.

Aspects relate to a nucleic acid encoding an antibody heavy chain or VH, wherein the nucleic acid has a nucleotide sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:21 or 23. Further aspects relate to a nucleic acid encoding an antibody light chain or VL, wherein the nucleic acid has a nucleotide sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:24 or 26. The nucleic acids of the disclosure may be DNA or RNA.

The VH in polypeptides, antibodies, antigen binding fragments, CARs, and scFvs of the disclosure comprises a heavy chain framework region (HFR) 1, HFR2, HFR3, and HFR4. The VH may comprise, from N-terminus to C-terminus HFR1, HCDR1, HFR2, HCDR2, HFR3, HCDR3, and HFR4. The VL in polypeptides, antibodies, antigen binding fragments, CARs, and scFvs of the disclosure comprises a light chain framework region (LFR) 1, LFR2, LFR3, and LFR4. The LH may comprise, from N-terminus to C-terminus LFR1, LCDR1, LFR2, LCDR2, LFR3, LCDR3, and LFR4. The HFR1 may have an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:7. The HFR2 may have an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:8. The HFR3 may have an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:9. The HFR4 may have an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:10. The LFR1 may have an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:17. The LFR2 may have an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:18. The LFR3 may have an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:19. The LFR4 may have an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:20. In some aspects, the VH in polypeptides, antibodies, antigen binding fragments, CARs, and scFvs of the disclosure comprises a HFR1, HFR2, HFR3, and HFR4 or SEQ ID NOS:7-10, respectively. In some aspects, the VL in polypeptides, antibodies, antigen binding fragments, CARs, and scFvs of the disclosure comprises a LFR1, LFR2, LFR3, and LFR4 or SEQ ID NOS:17-20, respectively.

The VH in polypeptides, antibodies, antigen binding fragments, CARs, and scFvs of the disclosure may comprise an amino acid sequence with at least 80% sequence identity to SEQ ID NO:3 and/or the VL in polypeptides, antibodies, antigen binding fragments, CARs, and scFvs of the disclosure may comprise an amino acid sequence with at least 80% sequence identity to SEQ ID NO:13. In some aspects, the VH comprises an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:3. In some aspects, the VH comprises or consists of SEQ ID NO:3. In some aspects, the VL comprises an amino acid acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:33. In some aspects, the VL comprises or consists of SEQ ID NO:13.

In some aspects, the antibody, antigen binding fragment, polypeptide, CAR, or scFv comprises an amino acid sequence with at least 70% sequence identity to one of SEQ ID NOS:1-20 and/or an amino acid sequence with 1 substitution relative to SEQ ID NOS:1-20. In some aspects, the antibody, antigen binding fragment, polypeptide, CAR, or scFv comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:1-20 and/or an amino acid sequence having, having at least, or having at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 substitution relative to SEQ ID NOS:1-20.

In some aspects, the antibody, antigen binding fragment, scFv, CAR, or polypeptide of the disclosure comprises a heavy chain and/or a light chain. In some aspects, the heavy chain comprises an amino acid sequence with at least 70% sequence identity to SEQ ID NO:1. In some aspects, the heavy chain comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:1. In some aspects, the heavy chain comprises or consists of SEQ ID NO:1. In some aspects, the light chain comprises an amino acid sequence with at least 70% sequence identity to SEQ ID NO:11. In some aspects, the light chain comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:11. In some aspects, the light chain comprises or consists of SEQ ID NO:11.

In some aspects, the antibody, antigen binding fragment, scFv, CAR, or polypeptide of the disclosure comprises or further comprises a signal peptide. In some aspects, the signal peptide comprises SEQ ID NO:2 or 12 or an amino acid sequence having at least 80% sequence identity to SEQ ID NO:2 or 12. In some aspects, the signal peptide comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:2 or 12.

In aspects of the disclosure, the CDR is a CDR having an amino acid sequence that is obtained or determined by the Chothia method. In aspects of the disclosure, the CDR is a CDR having an amino acid sequence that is obtained or determined by the Kabat method. In aspects of the disclosure, the CDR is a CDR having an amino acid sequence that is obtained or determined by the IMGT method.

The antibody or antigen binding fragment of the disclosure may be human, chimeric, or humanized. In some aspects, the antibody, or antigen binding fragment binds B7-H3 with a K_{D} of about 10⁻⁶ nM to about 10⁻¹² pM. In some aspects, the antibody, or antigen binding fragment binds B7-H3 with a K_{D} of about, a K_{D} of at least, or a K_{D} of at most 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻¹⁸ (or any derivable range therein) μM, nM, or pM. The antibody, antigen binding fragment, scFv, or polypeptide may be further defined as a blocking antibody, antigen binding fragment, scFv, or polypeptide. The antibody, antigen binding fragment, scFv, or polypeptide may be further defined as a neutralizing antibody, antigen binding fragment, scFv, or polypeptide. In some aspects, the antibody, antigen binding fragment, scFv, or polypeptide is further defined as a human, humanized, recombinant, chimeric, derivative, veneered antibody, antigen binding fragment, scFv, or polypeptide. In some aspects, the antibody, antigen binding fragment, or polypeptide is further defined as a diabody, a monoclonal antibody or antigen binding fragment, a single domain antibody, or a single chain antibody. In some aspects, the antigen binding fragment is further defined as a single chain variable fragment (scFv), F(ab′)₂, Fab′, Fab, Fv, or rIgG. In some aspects, the antibody, antigen binding fragment, or polypeptide is operatively linked to a detectable label. Detectable labels are described herein.

In aspects of the disclosure, the antibody is a chimeric antibody. In some aspects, the chimeric antibody comprises a heavy chain comprising the amino acid sequence of SEQ ID NO:113 or an amino acid sequence with at least 70% sequence identity to SEQ ID NO:113 and a light chain comprising the amino acid sequence of SEQ ID NO:114 or an amino acid sequence with at least 70% sequence identity to SEQ ID NO:114. In some aspects, the chimeric antibody comprises a heavy chain comprising an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:113. In some aspects, the chimeric antibody comprises a light chain comprising an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:114.

Aspects of the disclosure also relate to multi-specific antibodies and polypeptides. Accordingly, aspects relate to bivalent or bispecific antibodies that comprise two antigen binding fragments, wherein the antigen binding fragment is two of the same antigen binding fragments or two different antigen binding fragments described herein. The disclosure also provides for multi-specific polypeptides. Aspects relate to polypeptides comprising at least 2, 3, 4, 5, or 6 antigen binding fragments. The antigen binding fragment may be at least 2, 3, r, 5, or 6 scFv, F(ab′)₂, Fab′, Fab, Fv, or rIgG, or combinations thereof.

Polypeptides of the disclosure may comprise antibodies, antigen binding fragments, scFvs, or CARs of the disclosure. In polypeptides of the disclosure, the VH may be amino proximal to the VL. In some aspects, the VL is amino proximal to the VH. The VH and the VL region may be on the same polypeptide. In some aspects, the VH and the VL are separated by a peptide linker. The linker may be a glycine-serine liner. In some aspects, the linker may be a linker described herein. The linker may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids, or any derivable range therein. Polypeptides and CARs of the disclosure may comprise or further comprise a second scFv. In some aspects, the second scFv specifically binds to CD3. Polypeptides of the disclosure may comprise or further comprise a CD3 binding region. In some aspects, the CD3 binding region is a scFv. The second scFv may be amino proximal to the first scFv. In some aspects, the first scFv is amino proximal to the second scFv.

The cytoplasmic region in the CARs of the disclosure may comprise or further comprise a co-stimulatory region between the transmembrane domain and the cytoplasmic region. In some aspects, the transmembrane domain comprises a transmembrane domain of CD28. The primary intracellular signaling domain may comprise a CD28 or CD3 zeta signaling domain. In some aspects, the CAR comprises a peptide spacer between the extracellular binding domain and the transmembrane domain. In some aspects, the peptide spacer comprises less than 50 amino acids. The peptide spacer may comprise, comprise at least, or comprise at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 amino acids, or any derivable range therein. In some aspects, the peptide spacer comprises more than 50 amino acids. In some aspects, the peptide spacer comprises the hinge region of an IgG molecule. In some aspects, the peptide spacer comprises the hinge and CH₂CH₃ region of an IgG molecule. In some aspects, the CAR is multispecific. In some aspects, the CAR is bispecific. The CAR may comprise an antigen binding domain, wherein the antigen binding domain is an anti-CD3, anti-CD56, anti-CD8 antigen binding domain, and combinations thereof.

In some aspects, the peptide is 13 amino acids in length or shorter. In some aspects, the peptide has at least, at most, exactly, or consists of 7, 8, 9, 10, 11, 12, 13, 14, or 15 amino acids (or any range derivable therein). In a specific aspect, the peptide consists of 9 amino acids. In some aspects, the peptide is immunogenic. The term immunogenic may refer to the production of an immune response, such as a protective immune response. In some aspects, the peptide is modified. In some aspects, the modification comprises conjugation to a molecule. The molecule may be an antibody, a lipid, an adjuvant, or a detection moiety (tag). In some aspects, the peptide comprises 100% sequence identity to a peptide of SEQ ID NO:105 or 109. Peptides of the disclosure also include those that have at least 90% sequence identity to a peptide of SEQ ID NO:105 or 109. The peptides of the disclosure may have 1, 2, or 3 substitutions relative to a peptide of SEQ ID NO:105 or 109. In some aspects, the peptide has at least or at most 1, 2, 3, 4, or 5 substitutions relative to a peptide of SEQ ID NO:105 or 109.

In some aspects, the pharmaceutical composition is formulated for parenteral administration, intravenous injection, intramuscular injection, inhalation, or subcutaneous injection. In some aspects, the peptide is comprised in a liposome, lipid-containing nanoparticle, or in a lipid-based carrier. In some aspects, the pharmaceutical preparation is formulated for injection or inhalation as a nasal spray. In some aspects, the compositions of the disclosure are formulated as a vaccine. In some aspects, the composition further comprises an adjuvant.

In some aspects regarding the dendritic cells of the disclosure, the dendritic cell comprises a mature dendritic cell. In some aspects, the cell is a cell with an HLA-A type. The HLA may be a HLA-A, HLA-B, or HLA-C. In some aspects, the cell is an HLA-A3 or HLA-A11 type. In some aspects, the cell is an HLA-A01, HLA-A02, HLA-A24, HLA-B07, HLA-B08, HLA-B15, or HLA-B40. In some aspects, the method further comprises isolating the expressed peptide or polypeptide. In some aspects, the T cell comprises a CD8+ T cell. In some aspects, the T cell is a CD4+ T cell, a Th1, Th2, Th17, Th9, or Tfh T cell, a cytotoxic T cell, a memory T cell, a central memory T cell, or an effector memory T cell.

In method aspects of the disclosure, contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface. In some aspects, the APCs are dendritic cells.

In aspects of the disclosure, the immune effector cells are T cells, peripheral blood lymphocytes, natural killer (NK) cells, invariant NK cells, or NKT cells. The immune effector cells may be ones that have been differentiated from mesenchymal stem cell (MSC) or induced pluripotent stem (iPS) cells. The T cell aspects include T cells that are further defined as CD8+ T cells, CD4+ T cells, or γδ T cells. In certain aspects, the T cells are cytotoxic T lymphocytes (CTLs).

Compositions of the disclosure, such as pharmaceutical compositions may comprise a pharmaceutical excipient, carrier, or molecule described herein, such as the antibodies, antigen binding fragments, polypeptides, scFvs, or CARs of the disclosure. In some aspects, the composition further comprises an adjuvant or an immunostimulator. Such adjuvants or immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments. Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure.

The cell may be a a human cell, B cell, T cell, Chinese hamster ovary, engineered cell, or immune cell. In some aspects, the cell is a natural killer (NK) cell. The immune cell may be a T cell. In some aspects, the T cell is a CD8+ T cell, CD4+ T cell, iNKT, or γδ T cell. In some aspects, the cell is an engineered cell. In some aspects, the engineered cell is an engineered T cell. In some aspects, the cell is a cell type or cell population described herein. Methods of the disclosure may comprise or further comprise culturing the cell under conditions that allow for expression of a polypeptide from the nucleic acid. Methods may comprise or further comprise isolating polypeptides expressed from the cells. Methods may comprise or further comprise analyzing polypeptides isolated from the cells. The analysis may include binding assays, such as binding assays to B7-H3, FACS analsysis, ELISA, western blot, or immunoassays.

In aspects of the disclosure, the subject or patient may be a human subject or a human patient. In some aspects, the subject or patient is a non-human animal. In some aspects, the non-human animal is a bat, monkey, camel, rat, mouse, rabbit, goat, chicken, bird, cat, dog, The subject may further be defined as an at-risk subject. The subject may be one that has been diagnosed with a cancer. The cancer may comprise leukemia. In some aspects, the cancer comprises acute myelogenous leukemia. In some aspects, cells, such as cells of the disclosure, are administered to the subject and wherein the cells are autologous. In some aspects, the cells, such as cells of the disclosure, are administered to the subject and wherein the cells are allogenic. In some aspects, the subject has previously been treated for the cancer. In some aspects, the subject has been determined to be resistant to the previous treatment. In some aspects, the method further comprises the administration of an additional therapy, such as an additional anticancer treatment. In some aspects, the additional therapy comprises a chemotherapy. In some aspects, the chemotherapy comprises cytarabine and/or daunorubicin. Further anticancer treatments include anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy and a biological therapy. In some aspects, the additional therapy comprises a targeted therapy. The target therapy may comprise one or more of a FLT3 inhibitor, IDH inhibitor, BCL-2 inhibitor, or a hedgehog pathway inhibitor. In some aspects, the targeted therapy comprises one or more of midostaurin, gilteritinib, ivosidenib, enasidenib, gemtuzumab ozogamicin, venetoclax, and glasdegib. In one aspect, the targeted therapy comprises venetoclax. In some aspects, the combination of the antibody or antigen binding fragment, polypeptide, CAR, composition, or cell and the additional therapy enhances NK cell-mediated killing of cancer cells. In some aspects, the additional therapy comprises an additional therapy described herein. The cancer may be further defined as stage I, II, III, or IV cancer. The cancer may comprise a solid tumor. The cancer may comprise metastatic and/or recurrent cancer. In some aspects, the cancer comprises non-metastatic cancer. In some aspects, the cancer is a B7H3-positive cancer. In some aspects, the cancer is a B7-H3^{high}-expressing cancer. In some aspects, the subject has been determined to have B7H3-positive cancer cells.

In some aspects, the cancer comprises a blood cancer. In some aspects, the cancer comprises a hematologic malignancy. In some aspects, the hematologic malignancy comprises Non-Hodgkin lymphoma, leukemia, multiple myeloma, or Hodgkin lymphoma. The leukemia may include acute lymphoblastic leukemia (ALL), acute myelogenous leukemia (AML), chronic myelogenous leukemia (CML), chronic lymphocytic leukemia (CLL), or hairy cell leukemia. In some aspects, the leukemia includes acute myelogenous leukemia, acute lymphoblastic leukemia, chronic myelogenous leukemia, chronic lymphocytic leukemia, hairy cell leukemia, large granular lymphocytic leukemia, chronic neutrophilic leukemia, Chronic eosinophilic leukemia, chronic monocytic leukemia, prolymphocytic leukemia, T-cell large granular lymphocytic leukemia, aggressive NK-cell leukemia, aggressive NK-cell leukemia, adult T-cell leukemia/lymphoma, sezary syndrome, myelodysplastic syndrome, or myeloproliferative disorders.

In some aspects, the cancer comprises breast cancer. The breast cancer may comprise a B7-H3 expressing breast cancer. In some aspects, the breast cancer comprises a B7-H3^{high}-expressing breast cancer. A B7-H3^{high}-expressing breast cancer is defined as one that has high expression of B7H3 compared to a control. In some aspects, the breast cancer comprises basal, luminal A, luminal B, triple negative breast cancer, or Her2-enriched breast cancer. In some aspects, the breast cancer excludes basal, luminal A, luminal B, triple negative breast cancer, or Her2-enriched breast cancer. The subject may be one that has been determined to have high expression of B7-H3 in a biological sample from the subject compred to a control. The control may comprise the level of expression of B7-H3 in non-cancerous cells. In some aspects, the biological sample from the subject comprises breast cancer cells and wherein the control comprises the level of expression of B7-H3 in non-cancerous mammary ducts.

In some aspects of the disclosure, the method further comprises incubating the antibody, antigen binding fragment, or polypeptide under conditions that allow for the binding of the antibody, antigen binding fragment, or polypeptide to antigens in the biological sample or extract thereof. In some aspects, the method further comprises detecting the binding of an antigen to the antibody, antigen binding fragment, or polypeptide. In some aspects, the method further comprises contacting the biological sample with at least one capture antibody, antigen, or polypeptide. The at least one capture antibody, antigen binding fragment, or polypeptide may be an antibody, polypeptide, or antigen binding fragment of the disclosure. In some aspects, the capture antibody is linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. In some aspects, the biological sample comprises a blood sample, urine sample, fecal sample, or nasopharyngeal sample. In aspects of the disclosure, the at least one antibody, antigen binding fragment, or polypeptide may be operatively linked to a detectable label. In some aspects, the method further comprises incubating the antibody, antigen binding fragment, or polypeptide under conditions that allow for the binding of the antibody, antigen binding fragment, or polypeptide to antigens in the biological sample or extract thereof. In some aspects, the method further comprises detecting the binding of an antigen to the antibody, antigen binding fragment, or polypeptide. In some aspects, the method further comprises contacting the biological sample with at least one capture antibody, antigen, or polypeptide. In some aspects, the biological sample comprises a blood sample, tissue sample, or a sample obtained by a biopsy. Methods of the disclosure may comprise or further comprise comparing the level of expression of B7-H3 to a control level of expression. In some aspects, the control comprises a cut-off. In some aspects, the control comprises the level of B7-H3 expression in a biological sample from a subject or the average level of B7-H3 expression in biological samples from subjects determined to not have cancer.

Methods of the disclosure may also comprise or further comprise sequencing one or more TCR genes from T cells bound with peptide and/or MHC. In some aspects, the method comprises or further comprises sequencing the TCR alpha and/or beta gene(s) from a TCR, such as a TCR that binds to a peptide of the disclosure. Methods may also comprise or further comprise grouping of lymphocyte interactions by paratope hotspots (GLIPH) analysis. This is further described in Glanville et al., Nature. 2017 Jul. 6; 547(7661): 94-98, which is herein incorporated by reference.

The compositions of the disclosure may be serum-free, mycoplasma-free, endotoxin-free, and/or sterile. The methods may further comprise culturing cells of the disclosure in media, incubating the cells at conditions that allow for the division of the cell, screening the cells, and/or freezing the cells. In some aspects, the method further comprises isolating the expressed peptide or polypeptide from a cell of the disclosure.

Methods of the disclosure may comprise or further comprise screening the dendritic cell for one or more cellular properties. In some aspects, the method further comprises contacting the cell with one or more cytokines or growth factors. The one or more cytokines or growth factors may comprise GM-CSF. In some aspects, the cellular property comprises cell surface expression of one or more of CD86, HLA, and CD14. In some aspects, the dendritic cell is derived from a CD34+ hematopoietic stem or progenitor cell.

The contacting in the methods of the disclosure may be further defined as co-culturing the starting population of immune effector cells with antigen presenting cells (APCs), wherein the APCs present the peptide on their surface. In a particular aspect, the APCs are dendritic cells. In some aspects, the dendritic cell is derived from a peripheral blood monocyte (PBMC). In some aspects, the dendritic cells are isolated from PBMCs. In some aspects, the dendritic cells are cells in which the DCs are derived from are isolated by leukaphereses.

Peptide-MHC (pMHC) complexes in the aspects of the disclosure may be made by contacting a peptide of the disclosure with a MHC complex. In some aspects, the peptide is expressed in the cell and binds to endogenous MHC complex to form a pMHC. In some aspects, peptide exchange is used to make the pMHC complex. For example, cleavable peptides, such as photocleavable peptides may be designed that bind to and stabilize the MHC. Cleavage of the peptide (eg. by irradiation for photocleavable peptides) dissociates the peptide from the HLA complex and results in an empty HLA complex that disintegrates rapidly, unless UV exposure is performed in the presence of a “rescue peptide.” Thus, the peptides of the disclosure may be used as “rescue peptides” in the peptide exchange procedure. Further aspects of the disclosure relate to pMHC complexes comprising a peptide of the disclosure. The pMHC complex may be operatively linked to a solid support or may be attached to a detectable moiety, such as a fluorescent molecule, a radioisotope, or an antibody. Further aspects of the disclosure relate to peptide-MHC multimeric complexes that include, include at least or include at most 1, 2, 3, 4, 5, or 6 peptide-MHC molecules operatively linked together. The linkage may be covalent, such as through a peptide bond, or non-covalent. In some aspects, pMHC molecules may be bound to a biotin molecule. Such pMHC molecules may be multimerized through binding to a streptavidin molecule. pMHC multermers may be used to detect antigen-specific T cells or TCR molecules that are in a composition or in a tissue. In some aspects, the multimers may be used to detect peptide-specific T cells in situ or in a biopsy sample. In further aspects, multimers may be bound to a solid support or deposited on a solid support, such as an array or slide. Cells may then be added to the slide, and detection of the binding between the pMHC multimer and cell may be conducted. Accordingly, the pMHC molecules and multimers of the disclosure may be used to detect and diagnose cancer in subjects or to determine immune responses in individuals with cancer.

In some aspects of the disclosure, obtaining, as defined in the methods described herein, comprises isolating the starting population of immune effector cells from peripheral blood mononuclear cells (PBMCs). In some aspects, the starting population of immune effector cells is obtained from a subject. The subject may be one that has a cancer, such as a peptide-specific cancer. In some aspects, the subject has been determined to have a cancer that expresses a peptide of the disclosure.

In some aspects, purifying further comprises generation of a clonal population of peptide-specific immune effector cells by limiting or serial dilution of sorted cells followed by expansion of individual clones by a rapid expansion protocol. In some aspects, methods of the disclosure comprise or further comprise cloning of a T cell receptor (TCR) from the clonal population of peptide-specific immune effector cells. In some aspects, the term isolating in the methods of the disclosure is defined as cloning of a T cell receptor (TCR) from the clonal population of peptide-specific immune effector cells. In some aspects, cloning of the TCR is cloning of a TCR alpha and a beta chain. In some aspects, the TCR is cloned using a 5′-Rapid amplification of cDNA ends (RACE) method. In some aspects, the TCR alpha and beta chains are cloned using a 5′-Rapid amplification of cDNA ends (RACE) method. In some aspects, the cloned TCR is subcloned into an expression vector. In some aspects, the expression vector comprises a linker domain between the TCR alpha sequence and TCR beta sequence. In some aspects, the expression vector is a retroviral or lentiviral vector. The vector may also be an expression vector described herein. The linker domain may comprise a sequence encoding one or more peptide cleavage sites. The one or more cleavage sites may be a Furin cleavage site and/or a P2A cleavage site. In some aspects, the TCR alpha sequence and TCR beta sequence are linked by an IRES sequence.

A host cell of the disclosure may be transduced with an expression vector to generate an engineered cell that expresses the TCR alpha and/or beta chains. In some aspects, the host cell is an immune cell. The immune cell may be a T cell and the engineered cell may be referred to as an engineered T cell. The T cell may be type of T cell described herein, such as a CD8+ T cell, CD4+ T cell, or γδ T cell. In some aspects, the starting population of immune effector cells is obtained from a subject having a cancer or a peptide-specific cancer and the host cell is allogeneic or autologous to the subject. In some aspects, the peptide-specific T cells are autologous or allogeneic. In some aspects, a population of CD4-positive or CD8-positive and peptide MHC tetramer-positive engineered T cells are purified from the transduced host cells. In some aspects, a clonal population of peptide-specific engineered T cells are generated by limiting or serial dilution followed by expansion of individual clones by a rapid expansion protocol. In some aspects, purifying in the methods of the disclosure is defined as purifying a population of CD4-positive or CD8-positive and peptide MHC tetramer-positive T cells from the immune effector cells following the co-culturing.

In some aspects, the peptide is linked to a solid support. In some aspects, the peptide is conjugated to the solid support or is bound to an antibody that is conjugated to the solid support. In some aspects, the solid support comprises a microplate, a bead, a glass surface, a slide, or a cell culture dish. In some aspects, the solid support comprises a nanofluidic chip. In some aspects, detecting T cell responses comprises detecting the binding of the peptide to the T cell or TCR. In some aspects, detecting T cell responses comprises an ELISA, ELISPOT, or a tetramer assay.

Kit aspects of the disclosure may comprise a peptide of the disclosure in a container. The peptide may be comprised in a pharmaceutical preparation. In some aspects, the pharmaceutical preparation is formulated for parenteral administration or inhalation. In some aspects, the peptide is comprised in a cell culture media.

The terms “protein,” “polypeptide,” and “peptide” are used interchangeably herein when referring to a gene product.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino proximal,” “N-terminus,” “amino terminus,” and the like as used herein are used to refer to order of the regions of the polypeptide. Furthermore, when something is N-terminal or amino proximal to a region it is not necessarily at the terminus (or end) of the entire polypeptide, but just at the N-terminus of the region or domain. Similarly, the terms “carboxy proximal,” “C-terminus,” “carboxy terminus,” and the like as used herein is used to refer to order of the regions of the polypeptide, and when something is C-terminal or carboxy proximal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment or aspect of this invention that is a polynucleotide encompasses both the double-stranded form and each of two complementary single-stranded forms known or predicted to make up the double-stranded form.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof” means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)2, Fab, Fv, rlgG, and scFv fragments. The term scFv refers to a single chain Fv antibody in which the variable domains of the heavy chain and of the light chain of a traditional two chain antibody have been joined to form one chain.

As used herein, the term “binding affinity” refers to the equilibrium constant for the reversible binding of two agents and is expressed as a dissociation constant (Kd). Binding affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

“Individual, “subject,” and “patient” are used interchangeably and can refer to a human or non-human.

“Treatment” or treating may refer to any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-H. B7-H3 is overexpressed in AML patients and associated with poor clinical outcomes. A. B7-H3 expression in AML patients and controls measured by flow cytometry; PBMCs were isolated from AML patients and healthy donors using Ficoll and stained with CD34 and B7-H3 (BioLegend; clone MIH42) antibody conjugated with PE and APC, respectively. B. B7-H3 expression on CD34⁺ and CD34⁻ subpopulations in AML patients and controls. C. B7-H3 expression in patients by European LeukemiaNet prognostic risk group, i.e., good, intermediate, and poor risk. D. Overall survival of AML patients based on high and low B7-H3 expression using receiver operating characteristic value as a cutoff. E. B7-H3 expression in AML lines HL-60, ThP-1, Kasumi-1, MOLM-13, MOLM-14, OCI-AML3, OCI-AML2, U937, and MV4-11; leukemic cells (0.5 to 1 million) were stained with B7-H3 (BioLegend; clone MIH42) antibody conjugated with APC and measured for B7-H3 expression using flow cytometry. F. mRNA expression of B7-H3 in FLT3-ITD-mutated and wild-type obtained from OSHU AML data set. Graph was generated using cBioPortal for Cancer Genomics data analysis software. Paired t-test was used for statistical analysis. G. Overall survival of AML patients based on low and high mRNA level of B7-H3. H. Disease-free survival of AML patients based on low and high mRNA level of B7-H3; same data set was used for survival analysis as in G. Statistical analysis: Wilcoxon rank sum test (A and B), Kruskal-Wallis test (C) Student unpaired t-test (F), log-rank test (D, G, and H); ***p<0.0001.

FIG. 2A-H. Knockdown of B7-H3 expression induced NK cell-mediated apoptosis. A. MV4-11 with knockdown of B7-H3 and scrambled shRNA as a control co-cultured in presence and absence of NK cells at 2:1 NK to target cell ratio. Leukemic cells are labeled with Cytolight red and simultaneously treated with annexin V green reagent. Time-lapse live-cell fluorescence imaging was performed using an Incucyte imaging system to assess apoptosis via annexin V staining every 1 hour for 12 hours. B. Bar diagram obtained at 12 hours showing percentage of annexin V positive in B7-H3 knockdown MV4-11 and scrambled shRNA control cells treated with and without NK cells at 2:1 ratio. C. Overview of cell death kinetic analysis in B7-H3 knockdown U937 cell lines and scrambled RNA as a control co-cultured in absence and presence of NK cells at 2:1 NK to target cell ratio; the inventors measured percentage of annexin V-positive every 1 hour for 12 hours. D. At 12 hours, bar diagram represents percentage of annexin V-positive in B7-H3 knockdown U937 and scrambled shRNA control cells treated with and without NK cells at 2:1 ratio. E-G. Dot plot showing B7-H3 expression in AML patients and controls using the inventors' own generated anti-B7-H3 mAbs (T-1A5, HEK5-1B3, and 58B1); PBMCs were isolated from AML patients and healthy donor (controls) using Ficoll and stained with primary anti-B7-H3 Abs T-1A5, HEK5-1B3, and 58B1 and secondary conjugated goat anti-mouse IgG-Alexa Fluor 647; DAPI was used to exclude dead cells, and B7-H3 expression was measured by flow cytometry. H. Overlay plot showing cell surface staining of B7-H3 in AML cell lines OCI-AML3, U937, MV4-11, and ThP-1 using the inventors' anti-B7-H3 Abs T-1A5A10, HEK5-1B3, and 58B1 measured by flow cytometry. Statistical Analysis: Student unpaired t-test (A-D); Wilcoxon rank sum test (E); ***p<0.0001.

FIG. 3A-E. Anti-B7-H3 mAbs T-1A5, HEK5-1B3, and 58B1 enhance NK cell-mediated apoptosis in AML. A. Representative images obtained at 0, 8, and 16 hours showing apoptosis in OCI-AML3 cells treated anti-B7-H3 antibodies and NK cells. OCI-AML3 cells were labeled with Cytolight red and annexin V. The overlap represents apoptosis in leukemic cells. The images were acquired by Incucyte live-cell imager over time. B. Overview of time-lapse fluorescent diagram represents apoptosis in OCI-AML3 cell lines treated with anti-B7-H3 mAbs T-1A5, HEK5-1B3, and 58B1 and NK cells measured every 1 hour for 16 hours C. Representative bar diagram at 16 hours timepoint showing percentage of annexin V binding in OCI-AML3 cells treated with anti-B7-H3 mAbs (1 μg/mL) and NK cells at 2:1 effector: target ratio. D and E. Similar experiments were performed using U937 cells to show the immunomodulatory effect of anti-B7-H3 mAbs in combination with NK cells. Statistical analysis: Student unpaired t-test.

FIG. 4A-F. Targeting B7-H3 inhibits AML growth in vivo via NK cells: A. Overlay plot showing B7-H3 expression in 4 different AML PDX models, cells stained with primary anti-B7-H3 mAbs and secondary conjugated antibody as control and measured it by flow cytometry. B. Treatment schema for AML PDX model; two million AML-PDX cells were injected in NSG mice via tail vein, and the mice were treated with mouse IgG1 or anti-B7-H3 mAbs T-1A5, HEK5-1B3, and 58B1 at 1 mg/kg twice a week via intraperitoneal injections. C. Mouse blood samples were analyzed weekly for human CD45⁺ cells by flow cytometer. When human CD45⁺ cells reached >95% or when mice became moribund (whichever happened first), mice were sacrified. D. Kaplan-Meier survival plot representing the overall survival rates in the mice treated with different anti-B7-H3 mAbs. E. Treatment schema for xenograft model; two million firefly luciferase GFP⁺ OCI-AML3 cells were injected in NSG mice via tail vein, and measured leukemia engraftment weekly by BLI system; when BLI reached leukemic cells 1.5×10⁹ photons/second, the mice were treated with anti-B7-H3 mAb T-1A5 or mouse IgG1 at 1 mg/kg once a week via intraperitoneal injections and NK cells (10×10⁶) twice a week via tail vein. F. Kaplan-Meier survival plot demonstrating the overall survival rates in the mice treated with mAb T-1A5 or mouse IgG and NK cells. Statistical analysis: log-rank test (D and F); **p<0.001, ***p<0.0001.

FIG. 5A-G. Characterization of ChT-1A5 and its induction of antibody-dependent cellular cytotoxicity in AML cell lines and primary cells. A. Binding kinetics of anti-B7-H3 mAbs T-1A5 and ChT-1A5 to full B7-H3 protein measured by surface plasmon resonance (Octet). B, C. Overview of time-lapse fluorescent diagram represents apoptosis induced by NK cell ADCC in U937 and MV4-11 cell lines treated with ChT-1A5 or rituximab at 1, 5, and 10 μg/mL in presence of NK cells measured every 1 hour for 30 hours at 8:1 effector: target ratio. D, E. Bar diagram at 28 hours showing percentage of annexin V binding in U937 and MV4-11 cells treated with ChT-1A5 or rituximab at 1, 5, and 10 μg/mL in presence of NK cells in 8:1 effector: target ratio. F. Cell death kinetics showing induction of apoptosis in primary AML cells (3 patients) treated with ChT-1A5 or rituximab at 1 μg/mL in presence of NK cells measured every 1 hour for 12, 12 and 36 hours respectively at 4:1 effector: target ratio. G. Bar diagram represents the percentage of cell death in primary AML cells treated with ChT-1A5 or rituximab 1 μg/mL in presence of NK cells at 4:1 effector: target ratio at 12, 12 and 36 hours respectively. *p<0.05, **p<0.001, ***p<0.0001. The bars in 5G represent, from left to right, Primary AML cells, Rituximab-1 ug/ml, CHT-1A5-1 ug/ml, NK+Rituximab-1 ug/ml, and NK+ChT-1A5-1 ug/ml.

FIG. 6A-F. Monoclonal antibodies T-1A5 and ChT-1A5 bind to the FG loop region of B7-H3. (homology modeling and peptide docking). A. Schematic representation of B7-H3 structural domains generated using Biorender software. B. Representation of different structural elements in B7-H3 amino acid sequence (SEQ ID NO:117) is shown. C. 3D modeling and docked complexes of 2 predicted poses of variable heavy (VH, upper panel) or variable light (VL, lower panel) chains of T-1A5 (surface representation) with FG loop in B7-H3. Three complementary determining regions (CDRs) in both VH and VL are shown. D. Epitope mapping of B7-H3 peptides (SEQ ID NOS:101-110) (5 amino acid overlap) with T-1A5 or chimeric ChT-1A5 mAbs. B7-H3 full protein was used as a control while peptide 5 and peptide 9 showed stronger KD values. E. Raw traces of peptide 5 and peptide 9 of B7-H3 interacting with T-1A5 and ChT-1A5. F. Peptide 5 and peptide 9 in the extracellular domain of B7-H3 model.

FIG. 7A-D. ChT-1A5 in combination with NK cells inhibits tumor growth via antibody-dependent cellular cytotoxicity mediated in AML PDX model. A. Treatment schema: two million AML PDX cells were injected in NSG mice via tail vein and mice were treated with ChT1A5 or rituximab at 1 mg/kg twice a week via intraperitoneal injections with NK cells (10×10⁶) via tail vein twice a week. B. Mouse blood samples were analyzed weekly for human CD45⁺ cells by flow cytometer. When human CD45⁺ cells reached >95% or when mice became moribund (whichever happened first), the mice were sacrfied. C. Body weight from leukemia-bearing mice treated with ChT-1A5 or rituximab and NK cells; Body weight of mice were measured once a week. D. Kaplan-Meier survival plot representing the overall survival rates in the mice treated with ChT-1A5 or rituximab and NK cells.

FIG. 8A-B. B7-H3 expression in AML cell lines and primary cells. A. Overlay plot of B7-H3 expression in different AML cancer cell lines was observed using flow cytometry with B7-H3 BioLegend antibody conjugated with APC. B. Gating strategy to measure B7-H3 expression in CD34-positive and -negative cells in AML patients and controls. The x-axis represents B7-H3 expression, and the y-axis represents CD34 expression.

FIG. 9A-F. B7-H3 knockdown efficiency in AML cells and B7-H3 antibody binding affinity in AML cell lines. A. B7-H3 mRNA knockdown efficacy in AML cell lines was assessed using RT-PCR in comparison to shRNA. Lentiviral-mediated shRNA was used for stable knockdown of B7-H3 expression in AML cell lines MV4-11 and U937. B. Overlay plot represents B7-H3 knockdown efficacy at protein level in MV4-11 and U937, assessed using flow cytometry. C. Dot plot represents activity of NK cells. Cells were stained with CD16-PE, NKG2D-APC and measured its expression in CD56-PE-Cy7-stained cells using flow cytometry. D. Overlay plot showing cell surface staining of B7-H3 in AML cell lines MOLM-13, MOLM-14, Kasumi-1, OCI-AML2, and HL-60 using the inventors' anti-B7-H3 mAbs T-1A5, HEK5-1B3, and 58B1 measured by flow cytometry. E. B7-H3 binding affinity measured by flow cytometry in 9 different AML cell lines using the inventors' anti-B7-H3 antibodies and BioLegend B7-H3. Each group of bars in 9D represents, from left to right, data for B7-H3, T-1A5, HEK5-183, and 58B1. F. RT-PCR was performed to analyze mRNA expression in 9 different AML cell lines. Relative fold increases in B7-H3 gene expression were normalized to GAPDH. Data are plotted as mean values with error bars representing standard error.

FIG. 10A-C. Effect of anti-B7-H3 monoclonal antibodies on NK cell-mediated apoptosis in THP-1 cell line. A. Representative images showing apoptosis in THP-1 cells treated with NK cells and anti-B7-H3 antibody. THP-1 cells were labeled with annexin V. The overlap represents apoptotic cells. The images were acquired by Incucyte live-cell imager over time. B. Overview of cell death kinetics analysis in ThP-1 cell lines treated with NK cells and anti-B7-H3 antibody. C. Representative bar diagram at 8-hour time point showing annexin V binding in ThP-1 cells treated with anti-B7-H3 antibody and NK cells. **p<0.0001. The bars in FIG. 10C represent data for ThP-1, IgG1, NK+IgG1, T-1A5, and NK+T-1A5, from left to right.

FIG. 11A-H. Binding affinity of T-1A5 and ChT-1A5 in AML cell lines and patient samples. A. Chimeric antibody structure: variable region from mouse Ab and constant region from human Ab sequence B. Spectrums of UHPLC-SEC for mAbs T-1A5 and ChT-1A5 showing the purity of the mAbs. C. Binding affinity of chimeric T-1A5 antibody against the antigen was determined using ELISA. D-E. Overlay plot and MFI showing binding of T-1A5 and ChT-1A5 in B7-H3 knockdown and scrambled OCI-AML3 cells measured by flow cytometry F. Cell death kinetics analysis was measured in OCI-AML3 cells treated with ChT-1A5 antibody or rituximab at 1, 5, and 10 μg/mL in presence and absence of NK cells (8:1 effector: target ratio) every 1 hour for 22 hours; OCI-AML3 cells were labeled with Cytolight red and annexin V. G. Bar diagram indicates the percentage of annexin V binding in OCI-AML3 cells treated with different concentrations of ChT-1A5 or rituximab mAbs and NK cells at 22 hour. H. Live-cell imaging at 0, 12, and 24 hour represents apoptosis in primary cells treated with NK cells and ChT-1A5 or rituximab at 1, 5, and 10 μg/mL. Cytolight red and annexin V was used to label primary. Overlap represents cells undergoing apoptosis. ***p<0.0001.

FIG. 12A-C. ChT-1A5 induced NK cell ADCC in AML cell lines but had no effect on healthy donors. A. OCI-AML3 cells were treated with ChT1-A5 mAb or rituximab at 1, 5, and 10 g/mL in presence and absence of NK cells (8:1 effector: target ratio); leukemic cells were labeled with Cytolight red and annexin V. Representative images show apoptosis in OCI-AML3 cells. The images were acquired by Incucyte live-cell imager over time. B-C. PBMCs derived from 3 different healthy donors (Controls) were treated with ChT1-A5 mAb or rituximab at 1, 5, and 10 μg/mL in presence and absence of NK cells (8:1 effector: target ratio); PBMCs were labeled with Cytolight red and annexin V. The bars in FIG. 12C represent data for Normal cells, Rituximab-1 ug/ml, ChT-1AS-1 ug/ml, NK+Rituximab-1 ug/ml, NK+ChT-1A5-1 ug/ml (from left to right).

FIG. 13. Binding affinity of anti-B7-H3 antibodies to B7-H3 peptides. Binding kinetics of anti-B7-H3 mAbs T-1A5 and ChT-1A5 to different B7-H3 peptides measured by surface plasmon resonance.

FIG. 14A-G. B7-H3 expression in primary breast tumors and cell lines. A, B7-H3 expression was analyzed in primary breast tumors using RNA-seq data derived from the TCGA (A, left) and progression-free (A, right) survival analyses of BC patients with high or low B7-H3 expression in the TCGA datasets over a 25-year follow-up period. B, B7-H3 expression in primary breast tumors using microarray data derived from the METABRIC (B, left) and relapse-free survival analyses (B, right) of BC patients with high or low B7-H3 expression in the METABRIC datasets over a 25-year follow-up period. C, B7-H3 mRNA expression in BC subtypes in METABRIC data set. D, Immunohistochemical analysis of B7-H3 expression in frozen primary tissues of TNBC patients (n=50) and adjacent normal tissue (n=23) using unconjugated anti-B7-H3 antibody (T-1A5). E and F, B7-H3 expression (H-score: 0-300; mean±SEM) in all TNBC tumor (n=50) and normal (n=23) tissues (D). Mean (±SEM) B7-H3 expression (H-score: 0-300) in matched tumor tissue and adjacent normal tissue (n=16) from TNBC patients (E). G, Relative B7-H3 mRNA expression in 12 BC cell lines (left). B7-H3 protein expression (mean fluorescence intensity) in 13 BC cell lines measured using flow cytometry. ***P<0.001; ****P<0.0001.

FIG. 15A-D. B7-H3 inhibits T-cell infiltration into primary breast tumors. A, Images at 40× magnification of matched TNBC tissue samples with high and low expression of B7-H3 with CD3+, CD4+, and CD8+ T-cell infiltration. Black arrows point to expression of B7-H3, CD3, CD4, and CD8 in representative images. B and C, Quantitative analysis showing percentage of positive nuclei and h-score for high B7-H3 expression with CD3+, CD4+, and CD8′ T cells in tumor tissue (top). The bottom panels show upregulated B7-H3 expression in tumor tissue matched to CD3+, CD4+, and CD8′ T-cell infiltration in the same tissue samples (percentage of positive nuclei and h-score). D and E, Quantitative analysis showing percentage of positive nuclei and h-score for low B7-H3 expression with CD3+, CD4+, and CD8′ T cells in tumor tissue (top). The bottom panels show downregulated B7-H3 expression in tumor tissue matched to CD3+, CD4+, and CD8′ T-cell infiltration in the same tissue samples (percentage of positive nuclei and h-score). *P<0.05; **P<0.01; ***P<0.001; ****P<0.0001.

FIG. 16A-J. B7-H3 knockdown enhances NK and T cell-mediated apoptosis. A-B, Flow cytometry analysis of B7-H3 protein expression in scrambled shRNA (control) vs. B7-H3-KD MDA-MB-231 (A) and HCC1395 (B) BC cell lines. C and D, B7-H3-KD and control MDA-MB-231 cells were co-cultured with or without NK cells at a 5:1 NK-to-target cell ratio. BC cells expressed RFP and were labeled with annexin V. Images were acquired by IncuCyte live-cell imager. Line graphs showing annexin v binding over time. E and F, Quantification of annexin V binding in B7-H3-KD and control MDA-MB-231 (E) and HCC1395 cells co-cultured with NK cells at 10-h time point. G and H, Time lapse imaging of B7-H3-KD MDA-MB-231 (G) and HCC1395 (H) cells and control cells co-cultured with T cells at 5:1 T-to-target cell ratio. I-J, At 14-h time point, bar graph shows annexin V binding in B7-H3-KD and control MDA-MB-231 (I) or HCC1395 (J) cells co-cultured with T cells. *P≤0.05; **P<0.01; ***P<0.001; ****P<0.0001.

FIG. 17A-H. Anti B7H3 antibody enhances NK and T cell mediated apoptosis in breast cancer: A-B, Overview of cell death kinetic analysis in MCF7 (A) HCC38 (B) cell lines treated with NK (E:T; 4:1) and anti-B7-H3 antibody (T-1A5). Breast cancer cells labeled with RFP and annexin V. C-D, Representative bar graphs showing annexin V binding in both cell lines treated with T-1A5 and NK cells at 8-hour. E-F, Overview of cell death kinetic analysis in MCF7 (C) HCC38 cell lines treated with T cells (E:T; 10:1) and anti-B7-H3 antibody (T-1A5). G-H, Representative bar graphs showing annexin V binding in both cell lines treated with T-1A5 and T cells at 7-hour. *p<0.01, ***p<⁰0.0⁰⁰¹.

FIG. 18A-D. Chimeric anti-B7-H3 antibody induces ADCC in BC cell lines. A and C, Time-lapse graph showing apoptosis in MCF7 (A) and HCC38 (C) cells treated with NK cells (E:T 8:1) and chT-1A5 antibody at 10 μg/mL. B and D, Representative bar diagrams showing annexin V binding in MCF7 (B) HCC38 (D) cells treated with chT-1A5 antibody and NK cells. The readings were taken at the 10-h time point. **P<0.01; ***P<0.001.

FIG. 19A-E. Monoclonal antibodies T-1A5 and chT-1A5 bind to the FG loop region of B7-H3. A, Schematic representation of B7-H3 structural domains generated using Biorender software. B, Representation of different structural elements in B7-H3 amino acid sequence (SEQ ID NO:117) is shown. C, 3D modeling and docked complexes of 2 predicted poses of variable heavy (VH, top row) or variable light (VL, bottom row) chains of T-1A5 (surface representation) with FG loop in B7-H3. Three complementary determining regions (CDRs) in both VH and VL are shown. D, Epitope mapping of B7-H3 peptides (5 amino acid overlap, red) with T-1A5 or chT-1A5 monoclonal antibodies. The full B7-H3 protein was used as a control, while peptide 5 and peptide 9 showed stronger KD values. E, Raw traces of peptide 5 and peptide 9 of B7-H3 interacting with T-1A5 and chT-1A5.

FIG. 20A-C. Anti-B7-H3 antibody (T-1A5) enhances ABT-199 induced apoptosis. (A) Time-lapse fluorescence data represent apoptosis induced by NK-cell. OCI-AML3 cells were treated with T-1A5 (4 μg/ml) and/or ABT-199 at 500 nM, 1 μM and 2 μM in the presence/absence of NK cells at an 4:1 NK:target ratio. Apoptosis was measured every hour for 30 hours. Leukemic cells were labeled with CytoLight red and apoptotic cells with annexin V. (B) Bar graph showing the percentage of annexin V binding at 15 hours in OCI-AML3 cells treated with T-1A5 and/or Abt-199 at 500 nM, 1 μM, and 2 μM in the presence or absence of NK cells in an 4:1 NK:target ratio. (C) Representative images obtained at 15 hours indicate apoptosis in OCI-AML3 cells treated with anti-B7-H3 antibody and/or NK cells. OCI-AML3 cells were labeled with CytoLight red and annexin V. The overlap represents apoptotic AML cells. The images were acquired by an Incucyte live-cell imager.

FIG. 21. Anti-B7-H3 antibody (T-1A5) enhances ABT-199 induced apoptosis. (A) Time-lapse fluorescence data represent apoptosis induced by NK-cell. OCI-AML3 cells were treated with T-1A5 (4 μg/ml) and/or ABT-199 at 500 nM, 1 pM and 2 pM in the presence/absence of NK cells at an 4:1 NK:target ratio. Apoptosis was measured every hour for 30 hours. Leukemic cells were labeled with CytoLight red and apoptotic cells with annexin V. (B) Bar graph showing the percentage of annexin V binding at 15 hours in THP1 cells treated with T-1A5 and/or Abt-199 at 500 nM, 1 μM, and 2 μM in the presence or absence of NK cells in an 4:1 NK:target ratio. (C) Representative images obtained at 15 hours indicate apoptosis in THP1 cells treated with anti-B7-H3 antibody and/or NK cells. OCI-AML3 cells were labeled with CytoLight red and annexin V. The overlap represents apoptotic AML cells. The images were acquired by an Incucyte live-cell imager.

### DETAILED DESCRIPTION

The inventors found elevated expression of B7-H3 in AML patients compared to healthy donors. Moreover, B7-H3 overexpression was positively correlated with AML progenitor cells (p<0.01). Clinically, higher B7-H3 expression was associated with poor outcomes. Furthermore, NK cell-mediated apoptosis was 3-fold higher in all B7-H3-knockdown cell lines. They observed an increase in NK cell-mediated apoptosis in the presence of anti-B7-H3 antibodies (p<0.01). Moreover, treatment with the T-1A5 antibody inhibited AML growth in vivo and prolonged survival of AML-bearing mice, suggesting that blocking B7-H3 suppresses its immunomodulatory function in vitro and in vivo. Using epitope mapping, the inventors identified T-1A5 antibody binding to the FG loop region of B7-H3. Finally, they observed that the chimeric T-1A5 antibody induced NK cell-mediated ADCC in primary AML cells, and dramatically extended the survival of leukemia bearing mice (p<0.001). The data shown in the examples establish that anti-B7-H3 antibody facilitates altered immunomodulation function and enhances ADCC in AML.

### I. Antibodies

Aspects of the disclosure relate to antibodies, antigen binding fragments thereof, or polypeptides capable of specifically binding to B7H3 (CD276).

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (x) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some aspects, bispecific antibodies can be biparatopic, wherein a bispecific antibody may specifically recognize a different epitope from the same antigen. In some aspects, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some aspects, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for aspects of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1, CDR-H2 and CDR-H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:

1) Computational predictions of the tertiary structure of the antibody/epitope binding interactions based on the chemical nature of the amino acid sequence of the antibody variable region and composition of the epitope.

2) Hydrogen-deuterium exchange and mass spectroscopy

3) Polypeptide fragmentation and peptide mapping approaches in which one generates multiple overlapping peptide fragments from the full length of the polypeptide and evaluates the binding affinity of these peptides for the epitope.

4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some aspects, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

Certain aspects relate to antibody fragments, such as antibody fragments that bind to a SARS-CoV-2 spike protein. The term functional antibody fragment includes antigen-binding fragments of an antibody that retain the ability to specifically bind to an antigen. These fragments are constituted of various arrangements of the variable region heavy chain (VH) and/or light chain (VL); and in some aspects, include constant region heavy chain 1 (CH1) and light chain (CL). In some aspects, they lack the Fc region constituted of heavy chain 2 (CH2) and 3 (CH3) domains. Aspects of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with Antibody CDRs & Scaffolding Domains that Display the CDRs

Antigen-binding peptide scaffolds, such as complementarity-determining regions (CDRs), are used to generate protein-binding molecules in accordance with the aspects. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain aspects may have an affinity constant (Ka) of about, at least about, or at most about 10⁶, 10⁷, 10⁸, 10⁹, or 10¹⁰ M or any range derivable therein. Similarly, in some aspects, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain aspects, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other aspects, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG@(Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### II. Antibody Production

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various aspects can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some aspects, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with aspects include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as 0-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### B. Fully Human Antibody Production

Methods are available for making fully human antibodies. Using fully human antibodies can minimize the immunogenic and allergic responses that may be caused by administering non-human monoclonal antibodies to humans as therapeutic agents. In one aspect, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH═CH— (cis and trans), —COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain aspects of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### III. Obtaining Encoded Antibodies

In some aspects, there are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### IV. Antibodys, Antigen Binding Fragments, Polypeptides, and CARs

### A. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some aspects, a signal peptide directs the nascent protein into the endoplasmic reticulum. This is essential if a receptor is to be glycosylated and anchored in the cell membrane. Generally, the signal peptide natively attached to the amino-terminal most component is used (e.g. in an scFv with orientation light chain-linker-heavy chain, the native signal of the light-chain is used).

In some aspects, the signal peptide is cleaved after passage of the endoplasmic reticulum (ER), i.e., is a cleavable signal peptide. In some aspects, a restriction site is at the carboxy end of the signal peptide to facilitate cleavage.

### B. Antigen Binding Domain

Polypeptides of the present disclosure may comprise one or more antigen binding domains. An “antigen binding domain” describes a region of a polypeptide capable of binding to an antigen under appropriate conditions. In some aspects, an antigen binding domain is a single-chain variable fragment (scFv) based on one or more antibodies (e.g., CD20 antibodies). In some aspects, an antigen binding domain comprise a variable heavy (VH) region and a variable light (VL) region, with the VH and VL regions being on the same polypeptide. In some aspects, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10−5M, 10−6M, 10−7M, 10⁻⁸M, 10⁻⁹M, 10⁻¹⁰M, 10⁻¹¹M, 10⁻¹²M, or 10⁻¹³M. In some aspects, the KD of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰ M, 10⁻¹¹M, 10⁻¹²M, or 10⁻¹³M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some aspects, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

In some aspects, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some aspects, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

### C. Peptide Spacer

A peptide spacer, such as an extracellular spacer may link an antigen-binding domain to a transmembrane domain. In some aspects, a peptide spacer is flexible enough to allow the antigen-binding domain to orient in different directions to facilitate antigen binding. In one aspect, the spacer comprises the hinge region from IgG. In some aspects, the spacer comprises or further comprises the CH2CH3 region of immunoglobulin and portions of CD3. In some aspects, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some aspects, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some aspects, the extracellular spacer consists of or comprises a hinge region from an immunoglobulin (e.g. IgG). Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of an extracellular spacer may have effects on the CAR's signaling activity and/or the CAR-T cells' expansion properties in response to antigen-stimulated CAR signaling. In some aspects, a shorter spacer such as less than 50, 45, 40, 30, 35, 30, 25, 20, 15, 14, 13, 12, 11, or 10 amino acids is used. In some aspects, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge region can include one of the following amino acid sequences:

Table: Exemplary Hinge Regions

| SEQUENCE | SEQ ID NO: |
|---|---|
| DKTHT | 27 |
| CPPC | 28 |
| CPEPKSCDTPPPCPR | 29 |
| ELKTPLGDTTHT | 30 |
| KSCDKTHTCP | 31 |
| KCCVDCP | 32 |
| KYGPPCP | 33 |
| EPKSCDKTHTCPPCP | 34 |
| ELKTPLGDTTHTCPRCP | 35 |
| SPNMVPHAHHAQ | 36 |
| ESKYGPPCPPCP | 37 |
| EPKSCDKTYTCPPCP | 38 |

The extracellular spacer can comprise an amino acid sequence of a human IgG1, IgG2, IgG3, or IgG4, hinge region. The extracellular spacer may also include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:38).

The extracellular spacer can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:39), or a variant thereof.

The extracellular spacer may comprise or further comprise a CH2 region. An exemplary CH2 region is APEFEGGPSVFLFPPKPKDTLMISRTPEVTCVVVDVSQEDPEVQFNWYVDGVEVHNAKTKPR EEQFQSTYRVVSVLTVLHQDWLNGKEYKCKVSNKGLPSSIEKTISKAK (SEQ ID NO:40). The extracellular spacer may comprise or further comprise a CH3 region. An exemplary CH3 region is GQPREPQVYTLPPSQEEMTKNQVSLTCLVKGFYPSDIAVEWESNGQPENNYKTTPPVLDSDG SFFLYSRLTVDKSRWQEGNVFSCSVMHEALHNHYTQKSLSLSLGK (SEQ ID NO:41).

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some aspects, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

### D. Transmembrane Domain

Polypeptides of the present disclosure may comprise a transmembrane domain. In some aspects, a transmembrane domain is a hydrophobic alpha helix that spans the membrane. Different transmembrane domains may result in different receptor stability.

In some aspects, the transmembrane domain is interposed between the extracellular spacer and the cytoplasmic region. In some aspects, the transmembrane domain is interposed between the extracellular spacer and one or more costimulatory regions. In some aspects, a linker is between the transmembrane domain and the one or more costimulatory regions.

Any transmembrane domain that provides for insertion of a polypeptide into the cell membrane of a eukaryotic (e.g., mammalian) cell may be suitable for use. In some aspects, the transmembrane domain is derived from CD28, CD8, CD4, CD3-zeta, CD134, or CD7.

Exemplary transmembrane domains useful in any of the aspects of the disclosure include those in the table below:

Table: Exemplary transmembrane domain sequences

| | | SEQ ID |
|---|---|---|
| Description | Sequence | NO: |
| CD28-derived | FWVLVVVGGVLACYSLLVTVAFIIFWV | 42 |
| CD8 beta derived | LGLLVAGVLVLLVSLGVAIHLCC | 43 |
| CD4 derived | ALIVLGGVAGLLLFIGLGIFFCVRC | 44 |
| CD3 zeta derived | LCYLLDGILFIYGVILTALFLRV | 45 |
| CD28 derived | WVLVVVGGVLACYSLLVTVAFIIFWV | 46 |
| CD134 (OX40) derived | VAAILGLGLVLGLLGPLAILLALYLL | 47 |
| CD7 derived | ALPAALAVISFLLGLGLGVACVLA | 48 |

### E. Cytoplasmic Region

After antigen recognition, receptors of the present disclosure may cluster and a signal transmitted to the cell through the cytoplasmic region. In some aspects, the costimulatory domains described herein are part of the cytoplasmic region. In some aspects, the cytoplasmic region comprises an intracellular signaling domain. An intracellular signaling domain may comprise a primary signaling domain and one or more costimulatory domains.

Cytoplasmic regions and/or costimulatiory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some aspects, the cytoplasmic region includes at least one (e.g., one, two, three, four, five, six, etc.) ITAM motif as described herein. In some aspects, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an FFAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

In some aspects, a suitable cytoplasmic region can comprise an ITAM motif-containing portion of the full length DAP12 amino acid sequence. In some aspects, the cytoplasmic region is derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). In some aspects, a suitable cytoplasmic region can comprise an ITAM motif-containing portion of the full length FCER1G amino acid sequence.

In some aspects, the cytoplasmic region is derived from T cell surface glycoprotein CD3 delta chain (also known as CD3D; CD3-DELTA; T3D; CD3 antigen, delta subunit; CD3 delta; CD38; CD3d antigen, delta polypeptide (TiT3 complex); OKT3, delta chain; T cell receptor T3 delta chain; T cell surface glycoprotein CD3 delta chain; etc.). In some aspects, a suitable cytoplasmic region can comprise an ITAM motif-containing portion of the full length CD3 delta amino acid sequence. In some aspects, the cytoplasmic region is derived from T cell surface glycoprotein CD3 epsilon chain (also known as CD3e, CD3ε; T cell surface antigen T3/Leu-4 epsilon chain, T cell surface glycoprotein CD3 epsilon chain, A1504783, CD3, CD3-epsilon, T3e, etc.). In some aspects, a suitable cytoplasmic region can comprise an ITAM motif-containing portion of the full length CD3 epsilon amino acid sequence. In some aspects, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). In some aspects, a suitable cytoplasmic region can comprise an ITAM motif-containing portion of the full length CD3 gamma amino acid sequence. In some aspects, the cytoplasmic region is derived from T cell surface glycoprotein CD3 zeta chain (also known as CD3Z, CD3ζ, T cell receptor T3 zeta chain, CD247, CD3-ZETA, CD3H, CD3Q, T3Z, TCRZ, etc.). In some aspects, a suitable cytoplasmic region can comprise an ITAM motif-containing portion of the full length CD3 zeta amino acid sequence.

In some aspects, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some aspects, a suitable cytoplasmic region can comprise an ITAM motif-containing portion of the full length CD79A amino acid sequence.

Specific exemplary cytoplasmic regions are known in the art and further shown in the table below.

Table: Cytoplasmic Regions

| SEQUENCE | SEQ ID NO: |
|---|---|
| MGGLEPCSRLLLLPLLLAVSGLRPVQAQAQSDCSCSTVSPGVLAGIVMGD | 49 |
| LVLTVLIALAVYFLGRLVPRGRGAAEAATRKQRITETESPYQELQGQRSD | |
| VYSDLNTQRPYYK | |
| MGGLEPCSRLLLLPLLLAVSGLRPVQAQAQSDCSCSTVSPGVLAGIVMGD | 50 |
| LVLTVLIALAVYFLGRLVPRGRGAAEATRKQRITETESPYQELQGQRSDV | |
| YSDLNTQRPYYK | |
| MGGLEPCSRLLLLPLLLAVSDCSCSTVSPGVLAGIVMGDLVLTVLIALAV | 51 |
| YFLGRLVPRGRGAAEAATRKQRITETESPYQELQGQRSDVYSDLNTQRPY | |
| YK | |
| MGGLEPCSRLLLLPLLLAVSDCSCSTVSPGVLAGIVMGDLVLTVLIALAV | 52 |
| YFLGRLVPRGRGAAEATRKQRITETESPYQELQGQRSDVYSDLNTQRPYY | |
| K | |
| MIPAVVLLLLLLVEQAAALGEPQLCYILDAILFLYGIVLTLLYCRLKIQVR | 53 |
| KAAITSYEKSDGVYTGLSTRNQETYETLKHEKPPQ | |
| DGVYTGLSTRNQETYETLKHE | 54 |
| MEHSTFLSGLVLATLLSQVSPFKIPIEELEDRVFVNCNTSITWVEGTVGTLL | 55 |
| SDITRLDLGKRILDPRGIYRCNGTDIYKDKESTVQVHYRMCQSCVELDPA | |
| TVAGIIVTDVIATLLLALGVFCFAGHETGRLSGAADTQALLRNDQVYQPL | |
| RDRDDAQYSHLGGNWARNK | |
| MEHSTFLSGLVLATLLSQVSPFKIPIEELEDRVFVNCNTSITWVEGTVGTLL | 56 |
| SDITRLDLGKRILDPRGIYRCNGTDIYKDKESTVQVHYRTADTQALLRND | |
| QVYQPLRDRDDAQYSHLGGNWARNK | |
| DQVYQPLRDRDDAQYSHLGGN | 57 |
| MQSGTHWRVLGLCLLSVGVWGQDGNEEMGGITQTPYKVSISGTTVILTC | 58 |
| PQYPGSEILWQHNDKNIGGDEDDKNIGSDEDHLSLKEFSELEQSGYYVCY | |
| PRGSKPEDANFYLYLRARVCENCMEMDVMSVATIVIVDICITGGLLLLVY | |
| YWSKNRKAKAKPVTRGAGAGGRQRGQNKERPPPVPNPDYEPIRKGQRD | |
| LYSGLNQRRI | |
| NPDYEPIRKGQRDLYSGLNQR | 59 |
| MEQGKGLAVLILAIILLQGTLAQSIKGNHLVKVYDYQEDGSVLLTCDAEA | 60 |
| KNITWFKDGKMIGFLTEDKKKWNLGSNAKDPRGMYQCKGSQNKSKPLQ | |
| VYYRMCQNCIELNAATISGFLFAEIVSIFVLAVGVYFIAGQDGVRQSRASD | |
| KQTLLPNDQLYQPLKDREDDQYSHLQGNQLRRN | |
| DQLYQPLKDREDDQYSHLQGN | 61 |
| MKWKALFTAAILQAQLPITEAQSFGLLDPKLCYLLDGILFIYGVILTALFLR | 62 |
| VKFSRSADAPAYQQGQNQLYNELNLGRREEYDVLDKRRGRDPEMGGKP | |
| RRKNPQEGLYNELQKDKMAEAYSEIGMKGERRRGKGHDGLYQGLSTAT | |
| KDTYDALHMQALPPR | |
| MKWKALFTAAILQAQLPITEAQSFGLLDPKLCYLLDGILFIYGVILTALFLR | 63 |
| VKFSRSADAPAYQQGQNQLYNELNLGRREEYDVLDKRRGRDPEMGGKP | |
| QRRKNPQEGLYNELQKDKMAEAYSEIGMKGERRRGKGHDGLYQGLSTA | |
| TKDTYDALHMQALPPR | |
| RVKFSRSADAPAYQQGQNQLYNELNLGRREEYDVLDKRRGRDPEMGGK | 64 |
| PRRKNPQEGLYNELQKDKMAEAYSEIGMKGERRRGKGHDGLYQGLSTA | |
| TKDTYDALHMQALPPR | |
| NQLYNELNLGRREEYDVLDKR | 65 |
| EGLYNELQKDKMAEAYSEIGMK | 66 |
| DGLYQGLSTATKDTYDALHMQ | 67 |
| MPGGPGVLQALPATIFLLFLLSAVYLGPGCQALWMHKVPASLMVSLGED | 68 |
| AHFQCPHNSSNNANVTWWRVLHGNYTWPPEFLGPGEDPNGTLIIQNVNK | |
| SHGGIYVCRVQEGNESYQQSCGTYLRVRQPPPRPFLDMGEGTKNRIITAE | |
| GIILLFCAVVPGTLLLFRKRWQNEKLGLDAGDEYEDENLYEGLNLDDCS | |
| MYEDISRGLQGTYQDVGSLNIGDVQLEKP | |
| MPGGPGVLQALPATIFLLFLLSAVYLGPGCQALWMHKVPASLMVSLGED | 69 |
| AHFQCPHNSSNNANVTWWRVLHGNYTWPPEFLGPGEDPNEPPPRPFLDM | |
| GEGTKNRIITAEGIILLFCAVVPGTLLLFRKRWQNEKLGLDAGDEYEDENL | |
| YEGLNLDDCSMYEDISRGLQGTYQDVGSLNIGDVQLEKP | |
| ENLYEGLNLDDCSMYEDISRG | 70 |
| FWVLVVVGGVLACYSLLVTVAFIIFWVRSKRSRLLHSDYMNMTPRRPGP | 71 |
| TRKHYQPYAPPRDFAAYRS | |

### F. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some aspects, the costimulatory region is derived from an intracellular portion of the transmembrane protein 4-1BB (also known as TNFRSF9; CD137; CDw137; ILA; etc.). In some aspects, the costimulatory region is derived from an intracellular portion of the transmembrane protein CD28 (also known as Tp44). In some aspects, the costimulatory region is derived from an intracellular portion of the transmembrane protein ICOS (also known as AILIM, CD278, and CVID1). In some aspects, the costimulatory region is derived from an intracellular portion of the transmembrane protein OX-40 (also known as TNFRSF4, RP5-902P8.3, ACT35, CD134, OX40, TXGP1L). In some aspects, the costimulatory region is derived from an intracellular portion of the transmembrane protein BTLA (also known as BTLA1 and CD272). In some aspects, the costimulatory region is derived from an intracellular portion of the transmembrane protein CD27 (also known as S 152, T14, TNFRSF7, and Tp55). In some aspects, the costimulatory region is derived from an intracellular portion of the transmembrane protein CD30 (also known as TNFRSF8, D1S166E, and Ki-1). In some aspects, the costimulatory region is derived from an intracellular portion of the transmembrane protein GITR (also known as TNFRSF18, RP5-902P8.2, AITR, CD357, and GITR-D). In some aspects, the costimulatory region derived from an intracellular portion of the transmembrane protein HVEM (also known as TNFRSF14, RP3-395M20.6, ATAR, CD270, HVEA, HVEM, LIGHTR, and TR2).

Specific exemplary co-stimulatory domains are represented by the amino acid sequences below:

Table: Co-stimulatory domains

| SEQUENCE | SEQ ID NO: |
|---|---|
| KRGRKKLLYIFKQPFMRPVQTTQEEDGCSCRFPEEEEGGCEL | 72 |
| FWVRSKRSRLLHSDYMNMTPRRPGPTRKHYQPYAPPRDFAAYRS | 73 |
| TKKKYSSSVHDPNGEYMFMRAVNTAKKSRLTDVTL | 74 |
| RRDQRLPPDAHKPPGGGSFRTPIQEEQADAHSTLAKI | 75 |
| CCLRRHQGKQNELSDTAGREINLVDAHLKSEQTEASTRQNSQVLLSETGI | 76 |
| YDNDPDLCFRMQEGSEVYSNPCLEENKPGIVYASLNHSVIGPNSRLARN | |
| VKEAPTEYASICVRS | |
| HQRRKYRSNKGESPVEPAEPCRYSCPREEEGSTIPIQEDYRKPEPACSP | 77 |
| RRACRKRIRQKLHLCYPVQTSQPKLELVDSRPRRSSTQLRSGASVTEPVA | 78 |
| EERGLMSQPLMETCHSVGAAYLESLPLQDASPAGGPSSPRDLPEPRVSTE | |
| HTNNKIEKIYIMKADTVIVGTVKAELPEGRGLAGPAEPELEEELEADHTP | |
| HYPEQETEPPLGSCSDVMLSVEEEGKEDPLPTAASGK | |
| HIWQLRSQCMWPRETQLLLEVPPSTEDARSCQFPEEERGERSAEEKGRL | 79 |
| GDLWV | |
| CVKRRKPRGDVVKVIVSVQRKRQEAEGEATVIEALQAPPDVTTVAVEET | 80 |
| IPSFTGRSPNH | |
| RSKRSRGGHSDYMNMTPRRPGPTRKHYQPYAPPRDFAAYRS | 81 |

### G. Detection Peptides

In some aspects, the polypeptides described herein may further comprise a detection peptide. Suitable detection peptides include hemagglutinin (HA; e.g., YPYDVPDYA (SEQ ID NO:82); FLAG (e.g., DYKDDDDK (SEQ ID NO:83); c-myc (e.g., EQKLISEEDL; SEQ ID NO:84), and the like. Other suitable detection peptides are known in the art.

### H. Peptide Linkers

In some aspects, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G)n, glycine-serine polymers (including, for example, (GS)n, (GSGGS—SEQ ID NO:115)n, (G4S)n and (GGGS—SEQ ID NO:116)n, where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:85), GGSGG (SEQ ID NO:86), GSGSG (SEQ ID NO:87), GSGGG (SEQ ID NO:88), GGGSG (SEQ ID NO:89), GSSSG (SEQ ID NO:90), and the like. In further aspects, the linker comprises (EAAAK)n, wherein n is an integer of at least one (SEQ ID NO:91). In some aspects, n is at least, at most, or exactly 1, 2, 3, 4, 5, 6, 7, 8, 9, or 10 (or any derivable range therein) (SEQ ID NOS:91-100).

### I. Additional Modifications and Polypeptide Enhancements

As used herein, a “protein” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes and antibody fragment described herein as well as antibody domains, such as HCDR1, HCDR2, HCDR3, LCDR1, LCDR2, LCDR3, HFRW1, HFRW2, HFRW3, HFRW4, LFRW1, LFRW2, LFRW3, LFRW4, VH, VL, CH, or CL.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NO:1-100.

In some aspects, the antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOS:1-20 or 27-100.

In some aspects, the antibody, antigen binding fragment, or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-20 or 27-100.

In some aspects, the antibody, antigen binding fragment, protein, or polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOS:1-20 or 27-100 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-20 or 27-100.

In some aspects there is a nucleic acid molecule, antibody, antigen binding fragment, protein, or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:1-100 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:1-100.

In some aspects, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 of SEQ ID NOS:1-20 or polypeptides of SEQ ID NOS:27-100 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, a polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to one of SEQ ID NOS:1-20. The CDR may be one that has been determined by Kabat, IMGT, or Chothia. In further aspects, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some aspects, a polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. In some aspects, the CDRs of SEQ ID NOS:4-6 and 14-16 may further comprise 1, 2, 3, 4, 5, or 6 additional amino acids at the amino or carboxy terminus of the CDR, The additional amino acids may be from the heavy and/or light chain framework regions of SEQ ID NOS:3 and 13, that are shown as immediately adjacent to the CDRs. Accordingly, aspects relate to polypeptides comprising an HCDR1 (i.e., CDR-H1), HCDR2 (i.e., CDR-H2), HCDR3 (i.e., CDR-H3), LCDR1 (i.e., CDR-L1), LCDR2 (i.e., CDR-L2), and/or LCDR3 (i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids of SEQ ID NOS:3 and/or 13 that are shown as immediately adjacent to the CDRs. Other aspects relate to antibodies comprising one or more CDRs, wherein the CDR is a fragment of one of SEQ ID NOS:4-6 and 14-16 and wherein the fragment lacks 1, 2, 3, 4, or 5 amino acids from the amino or carboxy end of the CDR. In some aspects, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some aspects, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further aspects, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other aspects, a polypeptide or protein comprises 1, 2, 3, 4, 5, or 6 CDRs from either or both of the light and heavy variable regions of SEQ ID NOS:3 and 13, and 1, 2, 3, 4, 5, or 6 CDRs may have 1, 2, and/or 3 amino acid changes with respect to these CDRs. In some aspects, parts or all of the antibody sequence outside the variable region have been humanized. A protein may comprise one or more polypeptides. In some aspects, a protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### V. Immunotherapies Using Peptides of the Disclosure

A peptide as described herein (e.g., a peptide of Table 2) may be used for immunotherapy of a cancer. For example, a peptide of Table 2 may be contacted with or used to stimulate a population of T cells to induce proliferation of the T cells that recognize or bind said peptide. In other aspects, a peptide of the disclosure may be administered to a subject, such as a human patient, to enhance the immune response of the subject against a cancer.

A peptide of the disclosure may be included in an active immunotherapy (e.g., a cancer vaccine) or a passive immunotherapy (e.g., an adoptive immunotherapy). Active immunotherapies include immunizing a subject with a purified peptide antigen or an immunodominant peptide (native or modified); alternatively, antigen presenting cells pulsed with a peptide of the disclosure (or transfected with genes encoding an antigen comprising the peptide) may be administered to a subject. The peptide may be modified or contain one or more mutations such as, e.g., a substitution mutation. Passive immunotherapies include adoptive immunotherapies. Adoptive immunotherapies generally involve administering cells to a subject, wherein the cells (e.g., cytotoxic T cells) have been sensitized in vitro to a peptide of the disclosure (see, e.g., U.S. Pat. No. 7,910,109).

In some aspects, flow cytometry may be used in the adoptive immunotherapy for rapid isolation of human tumor antigen-specific T-cell clones by using, e.g., T-cell receptor (TCR) VP antibodies in combination with carboxyfluorescein succinimidyl ester (CFSE)-based proliferation assay. See, e.g., Lee et al., J. Immunol. Methods, 331:13-26, 2008, which is incorporated by reference for all purposes. In some aspects, tetramer-guided cell sorting may be used such as, e.g., the methods described in Pollack, et al., J Immunother Cancer. 2014; 2: 36, which is herein incorporated by reference for all purposes. Various culture protocols are also known for adoptive immunotherapy and may be used in aspects of the disclosure. In some aspects, cells may be cultured in conditions which do not require the use of antigen presenting cells (e.g., Hida et al., Cancer Immunol. Immunotherapy, 51:219-228, 2002, which is incorporated by reference). In other aspects, T cells may be expanded under culture conditions that utilize antigen presenting cells, such as dendritic cells (Nestle et al., 1998, incorporated by reference), and in some aspects artificial antigen presenting cells may be used for this purpose (Maus et al., 2002 incorporated by reference). Additional methods for adoptive immunotherapy are disclosed in Dudley et al. (2003), which is incorporated by reference, that may be used with aspects of the current disclosure. Various methods are known and may be used for cloning and expanding human antigen-specific T cells (see, e.g., Riddell et al., 1990, which is herein incorporated by reference).

In certain aspects, the following protocol may be used to generate T cells that selectively recognize peptides of the disclosure. Peptide-specific T-cell lines may be generated from normal donors or HLA-restricted normal donors and patients using methods previously reported (Hida et al., 2002). Briefly, PBMCs (1×105 cells/well) can be stimulated with about 10 μg/ml of each peptide in quadruplicate in a 96-well, U-bottom-microculture plate (Corning Incorporated, Lowell, MA) in about 200 μl of culture medium. The culture medium may consist of 50% AIM-V medium (Invitrogen), 50% RPMI1640 medium (Invitrogen), 10% human AB serum (Valley Biomedical, Winchester, VA), and 100 IU/ml of interleukin-2 (IL-2). Cells may be restimulated with the corresponding peptide about every 3 days. After 5 stimulations, T cells from each well may be washed and incubated with T2 cells in the presence or absence of the corresponding peptide. After about 18 hours, the production of interferon (IFN)-γ may be determined in the supernatants by ELISA. T cells that secret large amounts of IFN-γ may be further expanded by a rapid expansion protocol (Riddell et al., 1990; Yee et al., 2002b).

In some aspects, an immunotherapy may utilize a peptide of the disclosure that is associated with a cell penetrator, such as a liposome or a cell penetrating peptide (CPP). Antigen presenting cells (such as dendritic cells) pulsed with peptides may be used to enhance antitumour immunity (Celluzzi et al., 1996; Young et al., 1996). Liposomes and CPPs are described in further detail below. In some aspects, an immunotherapy may utilize a nucleic acid encoding a peptide of the disclosure, wherein the nucleic acid is delivered, e.g., in a viral vector or non-viral vector. In some aspects, a peptide of the disclosure may be used in an immunotherapy to treat cancer in a mammalian subject, such as a human patient.

### VI. Applications of Antigenic Peptides

Various aspects are directed to development of and use of antigenic peptides that that are useful for treating and preventing certain cancers. In many aspects, antigenic peptides are produced by chemical synthesis or by molecular expression in a host cell. Peptides can be purified and utilized in a variety of applications including (but not limited to) assays to determine peptide immunogenicity, assays to determine recognition by T cells, peptide vaccines for treatment of cancer, development of modified TCRs of T cells, and development of antibodies.

Peptides can be synthesized chemically by a number of methods. One common method is to use solid-phase peptide synthesis (SPPS). Generally, SPPS is performed by repeating cycles of alternate N-terminal deprotection and coupling reactions, building peptides from the c-terminus to the n-terminus. The c-terminus of the first amino acid is coupled the resin, wherein then the amine is deprecated and then coupled with the free acid of the second amino acid. This cycle repeats until the peptide is synthesized.

Peptides can also be synthesized utilizing molecular tools and a host cell. Nucleic acid sequences corresponding with antigenic peptides can be synthesized. In some aspects, synthetic nucleic acids synthesized in in vitro synthesizers (e.g., phosphoramidite synthesizer), bacterial recombination system, or other suitable methods. Furthermore, synthesized nucleic acids can be purified and lyophilized, or kept stored in a biological system (e.g., bacteria, yeast). For use in a biological system, synthetic nucleic acid molecules can be inserted into a plasmid vector, or similar. A plasmid vector can also be an expression vector, wherein a suitable promoter and a suitable 3′-polyA tail is combined with the transcript sequence.

Aspects are also directed to expression vectors and expression systems that produce antigenic peptides or proteins. These expression systems can incorporate an expression vector to express transcripts and proteins in a suitable expression system. Typical expression systems include bacterial (e.g., E. coli), insect (e.g., SF9), yeast (e.g., S. cerevisiae), animal (e.g., CHO), or human (e.g., HEK 293) cell lines. RNA and/or protein molecules can be purified from these systems using standard biotechnology production procedures.

Assays to determine immunogenicity and/or TCR binding can be performed. One such as is the dextramer flow cytometry assay. Generally, custom-made HLA-matched MHC Class I dextramer:peptide (pMHC) complexes are developed or purchased (Immudex, Copenhagen, Denmark). T cells from peripheral blood mononuclear cells (PBMCs) or tumor-infiltrating lymphocytes (TILs) are incubated the pMHC complexes and stained, which are then run through a flow cytometer to determine if the peptide is capable of binding a TCR of a T cell.

The peptides of the disclosure can also be used to isolate and/or identify T-cell receptors that bind to the peptide. T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T cell receptor differ from the membrane-bound immunoglobulin that serves as the B-cell receptor: a T cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Cα domain, where the fold is unlike that of any other immunoglobulin-like domain. The half of the domain that is juxtaposed with the Cβ domain forms a β sheet similar to that found in other immunoglobulin-like domains, but the other half of the domain is formed of loosely packed strands and a short segment of a helix. The intramolecular disulfide bond, which in immunoglobulin-like domains normally joins two β strands, in a Cα domain joins a β strand to this segment of a helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T cell receptors have been solved to this level of resolution.

Aspects of the disclosure relate to engineered T cell receptors that bind a peptide of the disclosure, such as a peptide of Table 1. The term “engineered” refers to T cell receptors that have TCR variable regions grafted onto TCR constant regions to make a chimeric polypeptide that binds to peptides and antigens of the disclosure. In certain aspects, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some aspects, the TCR comprises non-TCR sequences. Accordingly, certain aspects relate to TCRs with sequences that are not from a TCR gene. In some aspects, the TCR is chimeric, in that it contains sequences normally found in a TCR gene, but contains sequences from at least two TCR genes that are not necessarily found together in nature.

### VII. ROC Analysis

In statistics, a receiver operating characteristic (ROC), or ROC curve, is a graphical plot that illustrates the performance of a binary classifier system as its discrimination threshold is varied. ROC analysis may be applied to determine a cut-off value or threshold setting of biomarker expression, such as the canonical value described herein. For example, patients with biological samples determined to have biomarker expression value above a certain cut-off threshold but below a higher cut-off threshold may be determined to have endometriosis. Patients with biological samples determined to have a biomarker expression level that surpasses the cut-off threshold may be determined to have a disease or condition such as multiple sclerosis. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1—specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### VIII. Gene and RNA Expression Levels

Methods disclosed herein include measuring expression of genes and/or RNAs (RNAs) such as messenger RNAs (mRNAs), micro RNAs (miRNAs) and noncoding RNAs (ncRNAs). Measurement of expression can be done by a number of processes known in the art. The process of measuring expression may begin by extracting RNA from a metastasis tissue sample. Extracted mRNA and/or ncRNA can be detected by hybridization (for example by means of Northern blot analysis or DNA or RNA arrays (microarrays) after converting RNA into labeled cDNA) and/or amplification by means of a enzymatic chain reaction. Quantitative or semi-quantitative enzymatic amplification methods such as polymerase chain reaction (PCR) or quantitative real-time RT-PCR or semi-quantitative RT-PCR techniques can be used. Suitable primers for amplification methods encompassed herein can be readily designed by a person skilled in the art. Other amplification methods include ligase chain reaction (LCR), transcription-mediated amplification (TMA), strand displacement amplification (SDA), isothermal amplification of nucleic acids, and nucleic acid sequence based amplification (NASBA). Expression levels of mRNAs and/or ncRNAs may also be measured by RNA sequencing methods known in the art. RNA sequencing methods may include mRNA-seq, total RNA-seq, targeted RNA-seq, small RNA-seq, single-cell RNA-seq, ultra-low-input RNA-seq, RNA exome capture sequencing, and ribosome profiling. Sequencing data may be processed an aligned using methods known in the art.

To normalize the expression values of one gene among different samples, comparing the mRNA and/or ncRNA level of interest in the samples from the subject object of study with a control RNA level is possible. As it is used herein, a “control RNA” is an RNA of a gene for which the expression level does not differ among different metastatic subtypes, for example a gene that is constitutively expressed in all types of cells. A control RNA is preferably an mRNA derived from a housekeeping gene encoding a protein that is constitutively expressed and carrying out essential cell functions. A known amount of a control RNA may be added to the sample(s) and the value measured for the level of the RNA of interest may be normalized to the value measured for the known amount of the control RNA. Normalization for some methods, such as for sequencing, may comprise calculating the reads per kilobase of transcript per million mapped reads (RPKM) for a gene of interest, or may comprise calculating the fragments per kilobase of transcript per million mapped reads (FPKM) for a gene of interest. Normalization methods may comprise calculating the log 2-transformed count per million (log-CPM). It can be appreciated to one skilled in the art that any method of normalization that accurately calculates the expression value of an RNA for comparison between samples may be used.

Methods disclosed herein may include comparing a measured expression level to a reference expression level. The term “reference expression level” refers to a value used as a reference for the values/data obtained from samples obtained from patients. The reference level can be an absolute value, a relative value, a value which has an upper and/or lower limit, a series of values, an average value, a median, a mean value, or a value expressed by reference to a control or reference value. A reference level can be based on the value obtained from an individual sample, such as, for example, a value obtained from a sample from the subject object of study but obtained at a previous point in time. The reference level can be based on a high number of samples, such as the levels obtained in a cohort of subjects having a particular characteristic. The reference level may be defined as the mean level of the patients in the cohort. For example, the reference expression level for a gene or RNA can be based on the mean expression level of the gene or RNA obtained from a number of patients who have SNF2 metastases. A reference level can be based on the expression levels of the markers to be compared obtained from samples from subjects who do not have a disease state or a particular phenotype. The person skilled in the art will see that the particular reference expression level can vary depending on the specific method to be performed.

Some aspects include determining that a measured expression level is higher than, lower than, increased relative to, decreased relative to, equal to, or within a predetermined amount of a reference expression level. In some aspects, a higher, lower, increased, or decreased expression level is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and some aspects include determining that the measured expression level is higher by a predetermined amount or lower by a predetermined amount than a reference level. In some aspects, a level of expression may be qualified as “low” or “high,” which indicates the patient expresses a certain gene or RNA at a level relative to a reference level or a level with a range of reference levels that are determined from multiple samples meeting particular criteria. The level or range of levels in multiple control samples is an example of this. In some aspects, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. This can be the case, for example, when a classifier is used to identify the molecular subtype of a metastasis. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of gene and/or RNA expression levels to a mean expression levels or a reference expression levels, the comparison is to be made on a gene-by-gene and RNA-by-RNA basis. For example, if the expression levels of gene A, gene B, and miRNA X in a patient's metastasis are measured, a comparison to mean expression levels in metastases of a cohort of patients would involve: comparing the expression level of gene A in the patient's metastasis with the mean expression level of gene A in metastases of the cohort of patients, comparing the expression level of gene B in the patient's metastasis with the mean expression level of gene B in metastases of the cohort of patients, and comparing the expression level of RNA X in the patient's metastasis with the mean expression level of RNA X in metastases of the cohort of patients. Comparisons that involve determining whether the expression level measured in a patient's metastasis is within a predetermined amount of a mean expression level or reference expression level are similarly done on a gene-by-gene and ncRNA-by-ncRNA basis, as applicable.

### IX. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, or immunofluorescence techniques to detect biomarker expression. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

In some aspects, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some aspects, the antibody need not be labeled, and the presence thereof can be detected using a labeled antibody, which binds to the antibody.

### X. Sample Preparation

In certain aspects, methods involve obtaining a sample from a subject or the subject may be one that has been analyzed, determined, or evaluated expression of a biomarker. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. The sample may be obtained from any source including but not limited to blood, serum, plasma, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XI. Additional Therapies

### A. Immunotherapy

In some aspects, the methods comprise administration of an additional therapy. In some aspects, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Checkpoint Inhibitors and Combination Treatment

Aspects of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDIO680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### B. Chemotherapies

In some aspects, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### C. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

### D. Surgery

In some aspects, the additional therapy comprises surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months (or any range derivable therein). These treatments may be of varying dosages as well.

### XII. Sequences

Polypeptide, antibody, and antigen binding fragment aspects are shown below in the following tables.

TABLE 1
Antibody and antigen binding aspects

| | | SEQ |
|---|---|---|
| | | ID |
| Description | Sequence | NO: |
| Heavy | QVQLQQSGAELVKPGASVKLSCKASGYTFTSYDINWVRQRPEQGLEW | 1 |
| Chain | IGWIFPGDDTTNYNENFKGKATLTADKSSSTAYMQLTRLTSEDSAVFF | |
| | CARSVGFDYWGQGTTLTVSSAKTTPPSVYPLAPGSAAQTNSMVTLGC | |
| | LVKGYFPEPVTVTWNSGSLSSGVHTFPAVLQSDLYTLSSSVTVPSSTWP | |
| | SETVTCNVAHPASSTKVDKKIVPRDCGCKPCICTVPEVSSVFIFPPKPKD | |
| | VLTITLTPKVTCVVVDISKDDPEVQFSWFVDDVEVHTAQTQPREEQFN | |
| | STFRSVSELPIMHQDWLNGKEFKCRVNSAAFPAPIEKTISKTKGRPKAP | |
| | QVYTIPPPKEQMAKDKVSLTCMITDFFPEDITVEWQWNGQPAENYKN | |
| | TQPIMDTDGSYFVYSKLNVQKSNWEAGNTFTCSVLHEGLHNHHTEKS | |
| | LSHSPGK | |
| Signal | MGWSCVFLFLLSVTAGVHS | 2 |
| VH | QVQLQQSGAELVKPGASVKLSCKASGYTFTSYDINWVRQRPEQGLEW | 3 |
| | IGWIFPGDDTTNYNENFKGKATLTADKSSSTAYMQLTRLTSEDSAVFF | |
| | CARSVGFDYWGQGTTLTVSS | |
| HCDR1 | SYDIN | 4 |
| HCDR2 | WIFPGDDTTNYNENFKG | 5 |
| HCDR3 | SVGFDY | 6 |
| HFR1 | QVQLQQSGAELVKPGASVKLSCKASGYTFT | 7 |
| HFR2 | WVRQRPEQGLEWIG | 8 |
| HFR3 | KATLTADKSSSTAYMQLTRLTSEDSAVFFCAR | 9 |
| HFR4 | WGQGTTLTVSS | 10 |
| Light Chain | QIVLIQSPAIMSASPGEKVTMTCSASSSISYMYWYQQKPGTSPKRWIFE | 11 |
| | TSKLASGVPARFSGSGSGTSYSLTISSMEAEDAATYYCHQRSSYPTFGG | |
| | GTKLEIKRADAAPTVSIFPPSSEQLTSGGASVVCFLNNFYPKDINVKWK | |
| | IDGSERQNGVLNSWTDQDSKDSTYSMSSTLTLTKDEYERHNSYTCEAT | |
| | HKTSTSPIVKSFNRNEC | |
| Signal | MDFQVRIFSFLLISASVILSRG | 12 |
| VL | QIVLIQSPAIMSASPGEKVTMTCSASSSISYMYWYQQKPGTSPKRWIFE | 13 |
| | TSKLASGVPARFSGSGSGTSYSLTISSMEAEDAATYYCHQRSSYPTFGG | |
| | GTKLEIK | |
| LCDR1 | SASSSISYMY | 14 |
| LCDR2 | ETSKLAS | 15 |
| LCDR3 | HQRSSYPT | 16 |
| LFR1 | QIVLIQSPAIMSASPGEKVTMTC | 17 |
| LFR2 | WYQQKPGTSPKRWIF | 18 |
| LFR3 | GVPARFSGSGSGTSYSLTISSMEAEDAATYYC | 19 |
| LFR4 | FGGGTKLEIK | 20 |
| Heavy | CAGGTTCAGCTGCAGCAGTCTGGAGCTGAACTGGTAAAGCCTGGGG | 21 |
| Chain | CTTCAGTGAAGTTGTCCTGCAAGGCTTCTGGCTACACCTTCACAAGT | |
| | TATGATATAAACTGGGTGAGGCAGAGGCCTGAACAGGGACTTGAG | |
| | TGGATTGGATGGATTTTTCCTGGAGATGATACTACTAACTACAATG | |
| | AGAATTTCAAGGGCAAGGCCACACTGACTGCAGACAAATCCTCCAG | |
| | CACAGCCTACATGCAGCTCACCAGGCTGACATCTGAGGACTCTGCT | |
| | GTCTTTTTCTGTGCAAGATCGGTGGGCTTTGACTACTGGGGCCAAG | |
| | GCACCACTCTCACAGTCTCCTCAGCCAAAACGACACCCCCATCTGT | |
| | CTATCCACTGGCCCCTGGATCTGCTGCCCAAACTAACTCCATGGTG | |
| | ACCCTGGGATGCCTGGTCAAGGGCTATTTCCCTGAGCCAGTGACAG | |
| | TGACCTGGAACTCTGGATCCCTGTCCAGCGGTGTGCACACCTTCCC | |
| | AGCTGTCCTGCAGTCTGACCTCTACACTCTGAGCAGCTCAGTGACT | |
| | GTCCCCTCCAGCACCTGGCCCAGCGAGACCGTCACCTGCAACGTTG | |
| | CCCACCCGGCCAGCAGCACCAAGGTGGACAAGAAAATTGTGCCCA | |
| | GGGATTGTGGTTGTAAGCCTTGCATATGTACAGTCCCAGAAGTATC | |
| | ATCTGTCTTCATCTTCCCCCCAAAGCCCAAGGATGTGCTCACCATTA | |
| | CTCTGACTCCTAAGGTCACGTGTGTTGTGGTAGACATCAGCAAGGA | |
| | TGATCCCGAGGTCCAGTTCAGCTGGTTTGTAGATGATGTGGAGGTG | |
| | CACACAGCTCAGACGCAACCCCGGGAGGAGCAGTTCAACAGCACT | |
| | TTCCGCTCAGTCAGTGAACTTCCCATCATGCACCAGGACTGGCTCA | |
| | ATGGCAAGGAGTTCAAATGCAGGGTCAACAGTGCAGCTTTCCCTGC | |
| | CCCCATCGAGAAAACCATCTCCAAAACCAAAGGCAGACCGAAGGC | |
| | TCCACAGGTGTACACCATTCCACCTCCCAAGGAGCAGATGGCCAAG | |
| | GATAAAGTCAGTCTGACCTGCATGATAACAGACTTCTTCCCTGAAG | |
| | ACATTACTGTGGAGTGGCAGTGGAATGGGCAGCCAGCGGAGAACT | |
| | ACAAGAACACTCAGCCCATCATGGACACAGATGGCTCTTACTTCGT | |
| | CTACAGCAAGCTCAATGTGCAGAAGAGCAACTGGGAGGCAGGAAA | |
| | TACTTTCACCTGCTCTGTGTTACATGAGGGCCTGCACAACCACCATA | |
| | CTGAGAAGAGCCTCTCCCACTCTCCTGGTAAA | |
| Signal | ATGGGATGGAGCTGTGTCTTTCTCTTCCTCCTGTCAGTAACTGCAGG | 22 |
| Sequence | TGTCCACTCC | |
| VH | CAGGTTCAGCTGCAGCAGTCTGGAGCTGAACTGGTAAAGCCTGGGG | 23 |
| | CTTCAGTGAAGTTGTCCTGCAAGGCTTCTGGCTACACCTTCACAAGT | |
| | TATGATATAAACTGGGTGAGGCAGAGGCCTGAACAGGGACTTGAG | |
| | TGGATTGGATGGATTTTTCCTGGAGATGATACTACTAACTACAATG | |
| | AGAATTTCAAGGGCAAGGCCACACTGACTGCAGACAAATCCTCCAG | |
| | CACAGCCTACATGCAGCTCACCAGGCTGACATCTGAGGACTCTGCT | |
| | GTCTTTTTCTGTGCAAGATCGGTGGGCTTTGACTACTGGGGCCAAG | |
| | GCACCACTCTCACAGTCTCCTCA | |
| Light Chain | CAAATTGTTCTCATCCAGTCTCCAGCAATCATGTCTGCATCTCCAGG | 24 |
| | GGAGAAGGTCACCATGACCTGCAGTGCCAGTTCAAGTATAAGTTAC | |
| | ATGTACTGGTACCAGCAGAAGCCAGGCACCTCCCCCAAAAGATGG | |
| | ATTTTTGAAACATCCAAACTGGCTTCTGGAGTCCCTGCTCGCTTCAG | |
| | TGGCAGTGGGTCTGGGACCTCTTATTCTCTCACAATCAGCAGCATG | |
| | GAGGCTGAAGATGCTGCCACTTATTACTGCCATCAGCGGAGTAGTT | |
| | ACCCCACGTTCGGAGGGGGGACCAAGCTGGAAATAAAACGGGCTG | |
| | ATGCTGCACCAACTGTATCCATCTTCCCACCATCCAGTGAGCAGTTA | |
| | ACATCTGGAGGTGCCTCAGTCGTGTGCTTCTTGAACAACTTCTACCC | |
| | CAAAGACATCAATGTCAAGTGGAAGATTGATGGCAGTGAACGACA | |
| | AAATGGCGTCCTGAACAGTTGGACTGATCAGGACAGCAAAGACAG | |
| | CACCTACAGCATGAGCAGCACCCTCACGTTGACCAAGGACGAGTAT | |
| | GAACGACATAACAGCTATACCTGTGAGGCCACTCACAAGACATCAA | |
| | CTTCACCCATTGTCAAGAGCTTCAACAGGAATGAGTGT | |
| Signal | ATGGATTTTCAGGTGCGGATTTTCAGCTTCCTGCTAATCAGTGCCTC | 25 |
| Sequence | AGTCATACTGTCCAGAGGA | |
| VL | CAAATTGTTCTCATCCAGTCTCCAGCAATCATGTCTGCATCTCCAGG | 26 |
| | GGAGAAGGTCACCATGACCTGCAGTGCCAGTTCAAGTATAAGTTAC | |
| | ATGTACTGGTACCAGCAGAAGCCAGGCACCTCCCCCAAAAGATGG | |
| | ATTTTTGAAACATCCAAACTGGCTTCTGGAGTCCCTGCTCGCTTCAG | |
| | TGGCAGTGGGTCTGGGACCTCTTATTCTCTCACAATCAGCAGCATG | |
| | GAGGCTGAAGATGCTGCCACTTATTACTGCCATCAGCGGAGTAGTT | |
| | ACCCCACGTTCGGAGGGGGGACCAAGCTGGAAATAAAA | |
| Heavy Chain- | ATGCAACTCCTGTCTTGCATTGCACTAAGTCTTGCACTTGTCACGAA | 111 |
| Chimeric | TTCGCAGGTTCAGCTGCAGCAGTCTGGAGCTGAACTGGTAAAGCCT | |
| antibody | GGGGCTTCAGTGAAGTTGTCCTGCAAGGCTTCTGGCTACACCTTCA | |
| | CAAGTTATGATATAAACTGGGTGAGGCAGAGGCCTGAACAGGGAC | |
| | TTGAGTGGATTGGATGGATTTTTCCTGGAGATGATACTACTAACTAC | |
| | AATGAGAATTTCAAGGGCAAGGCCACACTGACTGCAGACAAATCCT | |
| | CCAGCACAGCCTACATGCAGCTCACCAGGCTGACATCTGAGGACTC | |
| | TGCTGTCTTTTTCTGTGCAAGATCGGTGGGCTTTGACTACTGGGGCC | |
| | AAGGCACCACTCTCACAGTCTCCTCAGCTAGCACCAAGGGCCCATC | |
| | GGTCTTCCCCCTGGCACCCTCCTCCAAGAGCACCTCTGGGGGCACA | |
| | GCGGCCCTGGGCTGCCTGGTCAAGGACTACTTCCCCGAACCGGTGA | |
| | CGGTGTCGTGGAACTCAGGCGCCCTGACCAGCGGCGTGCACACCTT | |
| | CCCGGCTGTCCTACAGTCCTCAGGACTCTACTCCCTCAGCAGCGTG | |
| | GTGACCGTGCCCTCCAGCAGCTTGGGCACCCAGACCTACATCTGCA | |
| | ACGTGAATCACAAGCCCAGCAACACCAAGGTGGACAAGAAAGTTG | |
| | AGCCCAAATCTTGTGACAAAACTCACACATGCCCACCGTGCCCAGC | |
| | ACCTGAACTCCTGGGGGGACCGTCAGTCTTCCTCTTCCCCCCAAAA | |
| | CCCAAGGACACCCTCATGATCTCCCGGACCCCTGAGGTCACATGCG | |
| | TGGTGGTGGACGTGAGCCACGAAGACCCTGAGGTCAAGTTCAACTG | |
| | GTACGTGGACGGCGTGGAGGTGCATAATGCCAAGACAAAGCCGCG | |
| | GGAGGAGCAGTACAACAGCACGTACCGTGTGGTCAGCGTCCTCACC | |
| | GTCCTGCACCAGGACTGGCTGAATGGCAAGGAGTACAAGTGCAAG | |
| | GTCTCCAACAAAGCCCTCCCAGCCCCCATCGAGAAAACCATCTCCA | |
| | AAGCCAAAGGGCAGCCCCGAGAACCACAGGTGTACACCCTGCCCC | |
| | CATCCCGGGAGGAGATGACCAAGAACCAGGTCAGCCTGACCTGCCT | |
| | GGTCAAAGGCTTCTATCCCAGCGACATCGCCGTGGAGTGGGAGAGC | |
| | AATGGGCAGCCGGAGAACAACTACAAGACCACGCCTCCCGTGCTG | |
| | GACTCCGACGGCTCCTTCTTCCTCTACAGCAAGCTCACCGTGGACA | |
| | AGAGCAGGTGGCAGCAGGGGAACGTCTTCTCATGCTCCGTGATGCA | |
| | TGAGGCTCTGCACAACCACTACACGCAGAAGAGCCTCTCCCTGTCT | |
| | CCGGGTAAATGA | |
| Light Chain- | ATGCAACTCCTGTCTTGCATTGCACTAAGTCTTGCACTTGTCACGAA | 112 |
| Chimeric | TTCACAAATTGTTCTCATCCAGTCTCCAGCAATCATGTCTGCATCTC | |
| antibody | CAGGGGAGAAGGTCACCATGACCTGCAGTGCCAGTTCAAGTATAA | |
| | GTTACATGTACTGGTACCAGCAGAAGCCAGGCACCTCCCCCAAAAG | |
| | ATGGATTTTTGAAACATCCAAACTGGCTTCTGGAGTCCCTGCTCGCT | |
| | TCAGTGGCAGTGGGTCTGGGACCTCTTATTCTCTCACAATCAGCAG | |
| | CATGGAGGCTGAAGATGCTGCCACTTATTACTGCCATCAGCGGAGT | |
| | AGTTACCCCACGTTCGGAGGGGGGACCAAGCTGGAAATAAAACGT | |
| | ACGGTGGCTGCACCATCTGTCTTCATCTTCCCGCCATCTGATGAGCA | |
| | GTTGAAATCTGGAACTGCCTCTGTTGTGTGCCTGCTGAATAACTTCT | |
| | ATCCCAGAGAGGCCAAAGTACAGTGGAAGGTGGATAACGCCCTCC | |
| | AATCGGGTAACTCCCAGGAGAGTGTCACAGAGCAGGACAGCAAGG | |
| | ACAGCACCTACAGCCTCAGCAGCACCCTGACGCTGAGCAAAGCAG | |
| | ACTACGAGAAACACAAAGTCTACGCCTGCGAAGTCACCCATCAGG | |
| | GCCTGAGCTCGCCCGTCACAAAGAGCTTCAACAGGGGAGAGTGTTA | |
| | G | |
| Heavy Chain- | MQLLSCIALSLALVTNSQVQLQQSGAELVKPGASVKLSCKASGYTFTS | 113 |
| Chimeric | YDINWVRQRPEQGLEWIGWIFPGDDTTNYNENFKGKATLTADKSSST | |
| antibody | AYMQLTRLTSEDSAVFFCARSVGFDYWGQGTTLTVSSASTKGPSVFPL | |
| | APSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLTC | |
| | LVKGFYPSDIAVEWESNGQPENNYKTTPPVLDSDGSFFLYSKLTVDKS | |
| | RWQQGNVFSCSVMHEALHNHYTQKSLSLSPGK* | |
| Light Chain- | MQLLSCIALSLALVTNSQIVLIQSPAIMSASPGEKVTMTCSASSSISYMY | 114 |
| Chimeric | WYQQKPGTSPKRWIFETSKLASGVPARFSGSGSGTSYSLTISSMEAEDA | |
| antibody | ATYYCHQRSSYPTFGGGTKLEIKRTVAAPSVFIFPPSDEQLKSGTASVV | |
| | CLLNNFYPREAKVQWKVDNALQSGNSQESVTEQDSKDSTYSLSSTLTL | |
| | SKADYEKHKVYACEVTHQGLSSPVTKSFNRGEC* | |

TABLE 2
Peptide aspects

| Peptide # | Sequence | SEQ ID NO: |
|---|---|---|
| 1 | LVGTDATLCCSFSPEPGFSLAQLNL | 101 |
| 2 | AQLNLIWQLTDTKQLVHSFAEGQDQ | 102 |
| 3 | EGQDQGSAYANRTALFPDLLAQGNA | 103 |
| 4 | AQGNASLRLQRVRVADEGSFTCFVS | 104 |
| 5 | TCFVSIRDFGSAAVSLQVAAPYSKP | 105 |
| 6 | PYSKPSMTLEPNKDLRPGDTVTITC | 106 |
| 7 | VTITCSSYRGYPEAEVFWQDGQGVP | 107 |
| 8 | GQGVPLTGNVTTSQMANEQGLFDVH | 108 |
| 9 | LFDVHSVLRVVLGANGTYSCLVRNP | 109 |
| 10 | LVRNPVLQQDAHGSVTITGQPMTFP | 110 |

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### XIII. Nucleic Acids

In certain aspects, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred aspect, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### XIV. Methods for Modifying Genomic DNA

In certain aspects, the genomic DNA is modified either to include additional mutations, insertions, or deletions, or to integrate certain molecular constructs of the disclosure so that the constructs are expressed from the genomic DNA. In some aspects, a nucleic acid encoding a polypeptide of the disclosure is integrated into the genomic DNA of a cell. In some aspects, a nucleic acid is integrated into a cell via viral transduction, such as gene transfer by lentiviral or retroviral transduction. In some aspects, genomic DNA is modified by integration of nucleic acid encoding a polypeptide of the present disclosure (e.g., a CAR) into the genome of a host cell via a retroviral vector, a lentiviral vector, or an adeno-associated viral vector.

In some aspects, the integration is targeted integration. In some aspects, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T cell receptor alpha constant) locus. For instance, cells would first be electroporated with a ribonucleoprotein (RNP) complex consisting of Cas9 protein complexed with a single-guide RNA (sgRNA) targeting the TRAC (T cell receptor alpha constant) locus. Fifteen minutes post electroporation, the cells would be treated with AAV6 carrying the HDR template that encodes for the CAR. In another example, double stranded or single stranded DNA comprises the HDR template and is introduced into the cell via electroporation together with the RNP complex.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, (DC31 integrase, Bxbl-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRISPR/Cas-like endonucleases, I-Tevl nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

### XV. Polypeptide Expression

In some aspects, there are nucleic acid molecule encoding polypeptides, antibodies, or antigen binding fragments of the disclosure. The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### XVI. Administration of Therapeutic Compositions

The present disclosure includes methods for treating disease and modulating immune responses in a subject in need thereof. The disclosure includes cells that may be in the form of a pharmaceutical composition that can be used to induce or modify an immune response.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The therapeutic compositions and treatments disclosed herein may precede, be co-current with and/or follow another treatment or agent by intervals ranging from minutes to weeks. In aspects where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, pg/day, or mg/day or any range derivable therein. Furthermore, such doses can be administered at multiple times during a day, and/or on multiple days, weeks, or months.

In some aspects, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some aspects, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one aspect, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic compositions of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

The cancers amenable for treatment include, but are not limited to, tumors of all types, locations, sizes, and characteristics. In some aspects, the cancer comprises a solid tumor. In some aspects, the methods relate to reducing tumor volume or treating cancers that are recurrent and/or metastatic. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, lung cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### XVII. Detectable Labels

In some aspects of this disclosure, it will be useful to detectably or therapeutically label a Fab polypeptide or protein G Fab-binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### XVIII. Sample Preparation

In certain aspects, methods involve obtaining or evaluating a sample from a subject. The sample may include a sample obtained from any source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a esophageal or a suspected esophageal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XIX. Cells

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain aspects transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SY5Y, SK-MES-1, SK—N—SH, SL3, SW403, Stimulus-triggered Acquisition of Pluripotency (STAP) cell or derivate SW403, T-cells, THP-1, Tumor cells, U20S, U937, peripheral blood lymphocytes, expanded T cells, hematopoietic stem cells, or Vero cells. In some aspects, the cell is an immune cell. The immune cell may be an engineered T cell or engineered NK cell. The T cell may be a CD8′ T cell, CD4+ T cell, or γδ T cell and the engineered cell is an engineered T cell or engineered NK cell.

Certain aspects relate to cells comprising polypeptides or nucleic acids of the disclosure. In some aspects the cell is an immune cell or a T cell. “T cell” includes all types of immune cells expressing CD3 including T-helper cells, invariant natural killer T (iNKT) cells, cytotoxic T cells, T-regulatory cells (Treg) gamma-delta T cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### XX. Kits

Certain aspects of the present invention also concern kits containing compositions of the disclosure or compositions to implement methods of the disclosure. In some aspects, kits can be used to detect the presence of a BZH3 in a sample. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some aspects, a kit contains one or more polypeptides capable of binding to B7H3, including polypeptides disclosed herein. In some aspects, a kit comprises a detection pair. In some aspects, a kit comprises an enzyme. In some aspects, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit aspects.

Kits may further comprise instructions for use. For example, in some aspects, a kit comprises instructions for detecting B7H3 in a sample.

It is contemplated that any method or composition described herein can be implemented with respect to any other method or composition described herein and that different aspects may be combined. The claims originally filed are contemplated to cover claims that are multiply dependent on any filed claim or combination of filed claims.

### XXI. EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—B7-H3—Blocking Antibody Enhances Natural Killer Cell-Mediated Apoptosis in Acute Myeloid Leukemia Cells and Inhibits their Growth In Vivo

Immune checkpoint protein B7-H3 is overexpressed in various human malignancies including acute myeloid leukemia (AML). Although several monoclonal antibodies targeting B7-H3 have shown promising results in solid tumors, its effect on the AML immune microenvironment remains unexplored. The inventors hypothesized that targeting B7-H3 alters its immunomodulatory function and enhances immune cell-mediated killing of AML.

The inventors analyzed B7-H3 expression in AML patients (n=100) and healthy donors (n=20) and investigated B7-H3's immunomodulatory function by inhibition of its expression or with blocking antibodies. Assays of immune cell-mediated killing were performed by the Incucyte live-cell imaging system. The inventors identified a B7-H3-blocking antibody with high binding affinity for AML cells in vitro and in vivo and generated a human-mouse chimeric antibody against B7-H3. Epitope mapping was performed to identify the antibody-binding site of B7-H3 protein. Finally, the inventors evaluated the effect of the chimeric anti-B7-H3 antibody on natural killer (NK) cell-mediated antibody-dependent cellular cytotoxicity (ADCC) in vitro and in vivo.

The inventors found elevated expression of B7-H3 in AML patients compared to healthy donors. Moreover, B7-H3 overexpression was positively correlated with AML progenitor cells (p<0.01). Clinically, higher B7-H3 expression was associated with poor outcomes. Furthermore, NK cell-mediated apoptosis was 3-fold higher in all B7-H3-knockdown cell lines. The inventors observed an increase in NK cell-mediated apoptosis in the presence of anti-B7-H3 antibodies (p<0.01). Moreover, treatment with the T-1A5 antibody inhibited AML growth in vivo and prolonged survival of AML-bearing mice, suggesting that blocking B7-H3 suppresses its immunomodulatory function in vitro and in vivo. Using epitope mapping, the inventors identified T-1A5 antibody binding to the FG loop region of B7-H3. Finally, the inventors observed that the chimeric T-1A5 antibody induced NK cell-mediated ADCC in primary AML cells, and dramatically extended the survival of leukemia bearing mice (p<0.001).

This data establish that anti-B7-H3 antibody facilitates altered immunomodulation function and enhances ADCC in AML.

### A. Introduction

Acute myeloid leukemia (AML) is an aggressive malignancy characterized by a block in myeloid differentiation, leading to uncontrolled proliferation of myeloblasts in blood and bone marrow (1). AML is the most commonly reported leukemia in adults, with an incidence rate of 4.3 cases per 100,000 every year (2, 3). Despite recent advances in the development of targeted therapeutic approaches, AML remains an aggressive malignancy with very high relapse rates and is associated with poor overall survival (4). Evidently, there is a dire need to identify clinically relevant therapeutic targets in combination with standard chemotherapy for AML patients. One such approach that has recently gained momentum is cancer immunotherapy. Targeting immune checkpoint regulatory molecules in combination with standard chemotherapy has been shown to be more effective than single-agent chemotherapeutic regimens (5, 6).

In this study, the inventors hypothesize that targeting B7-H3 using mAbs activates immune cells against AML, inhibiting leukemia growth. The inventors measured the expression of B7-H3 in AML patients and studied its effect on overall survival and prognosis. Further, the inventors investigated the effect of novel B7-H3-blocking antibodies on immune cell-mediated killing of AML cells in vitro and in vivo using AML cell lines, xenografts, and patient-derived xenograft (PDX) models. Moreover, the inventors also identified the specific binding site on human B7-H3 protein that is responsible for its immunomodulatory effect.

### B. Methods

### 1. Cell Culture and Generation of NK Cells

a. AML Cell Culture and Patient Samples

The inventors purchased HL-60, Kasumi-1, THP-1, MV4-11, and U937 cells from ATCC and MOLM-13, MOLM-14, OCI-AML3, and OCI-AML2 cells from DSMZ. These cell lines were cultured in Roswell Park Memorial Institute 1640 media (Mediatech) with 10% fetal bovine serum (FBS) and 1% penicillin/streptomycin (Sigma Aldrich). Tests for Mycoplasma contamination of leukemic cells and HEK293T cells are performed in the inventors' laboratory every 4-6 months. AML patients were recruited over a period between February 2018 and March 2021 at the leukemia sample bank at MD Anderson Cancer Center. All patient samples were collected according to a protocol approved by the MD Anderson Cancer Center institutional review board (Protocol #PA18-0129). All study participants provided written informed consent per the Declaration of Helsinki. Peripheral blood mononuclear cells (PBMCs) from patients and healthy donors were cultured in Roswell Park Memorial Institute 1640 media containing 10% FBS and 1% penicillin/streptomycin.

b. Primary NK Cell Culture

Buffy coats from healthy individuals were purchased from the Gulf Coast Regional Blood Center (Houston, TX). PBMCs were isolated using LymphoSep density separation (LSM, Corning). Further, to generate NK cells, the inventors first depleted CD3+ T cells from PBMCs through negative magnetic selection using CD3 beads (#130-050-101, Miltenyi Biotec). The resulting cell fraction was co-cultured with irradiated (100 cGy) K562 antigen-presenting cells in a 1:1 ratio (PBMC:APC) in X-VIVO 10 medium (Lonza) supplemented with 10% heat-inactivated fresh plasma (from the Gulf Coast Regional Blood Center) and 1% penicillin/streptomycin (Sigma-Aldrich). Cultures were refreshed with half-volume media every 2-3 days with the addition of IL-15 (10 ng/ml) and IL-21 (25 ng/ml) (PeproTech) and re-stimulated with irradiated K562 in a ratio of 1:1 on day 7. On day 14, the expanded NK cells were collected for in vitro and in vivo experiments. The expression levels of NK cell-specific markers CD56 (#362510, BioLegend) and its activation markers CD16 (#302008, BioLegend) and NKG2D (#320808, BioLegend) were analyzed by flow cytometry.

### 2. Knockdown of B7-H3 Expression in AML Cells

Lentiviral-mediated short-hairpin RNA (shRNA) was used for stable knockdown of B7-H3 in the leukemia cell lines MV4-11 and U937. Lentiviral shRNA vector (TRC-Hs 1.0, Clone ID: TRCN0000128062; sequence: 5′-AAAGGCCAGTATTAAGGCTAG-3′ SEQ ID NO:118) was purchased from GE Healthcare Dharmacon. Lentiviral pLKO.1 empty vector (#RHS4080, GE Healthcare Dharmacon) was used as a control. The virus was generated as described before (31). The abovementioned leukemia cell lines were transduced for 24 hours and then selected using puromycin (0.5 μg/mL) for 3 days. The B7-H3 knockdown efficacy was determined by measuring mRNA and protein expression using quantitative real-time polymerase chain reaction (RT-PCR) and flow cytometry, respectively.

3. Evaluation of B7-H3 Protein and mRNA Expression in Primary AML Cells and Cell Lines

Protein expression: The inventors performed flow cytometry to measure B7-H3 protein expression in AML cell lines, patient samples and healthy donor-derived PBMCs. Briefly, 1×10⁶ cells were washed twice with flow cytometry buffer (phosphate-buffered saline [PBS] plus 2% FBS), incubated in dark with anti-B7-H3-APC (#351006, BioLegend) and anti-CD34-PE (#343506, BioLegend) antibodies for 30 minutes on ice, washed in flow buffer, and counterstained with DAPI (0.5 g/mL; Thermo Fisher Scientific) to exclude any dead cells. To measure B7-H3 expression using unconjugated antibodies, including T-1A5, HEK5-1B3, and 58B1, the inventors used an indirect staining method as described before (32). Briefly, after the primary antibody incubation, the cells were washed twice and incubated with goat anti-mouse IgG-Alexa Fluor 647 (#A21235, Life Technologies) secondary antibody for 30 minutes. Finally, the cells were washed with DAPI (0.5 μg/mL; Thermo Fisher Scientific) containing FACS buffer to exclude any dead cells. The data were acquired on BD FACSCanto and LSR II (BD Biosciences) and Gallios (Beckman Coulter) flow cytometers. For each sample, a minimum of 10,000 events were acquired, and data were analyzed using FlowJo software v10 (FlowJo, LLC).

mRNA expression: RNA extraction was performed using a RNeasy Mini Kit (Qiagen) following the manufacturer's instructions. The cDNA for each sample was synthesized using 1 μg of RNA and SuperScript IV VILO Master Mix (Invitrogen). RT-PCR was performed with a QuantStudio 3 (Applied Biosystems) instrument using TaqMan Fast Universal PCR Master Mix (Applied Biosystems) as described previously (33). All samples were run in triplicates. The relative fold increase of specific RNA was calculated by the comparative cycle of threshold detection method, and values were normalized to glyceraldehyde 3-phosphate dehydrogenase (GAPDH). Fold changes in gene expression were calculated using the 2-ddCt method. The primers used were Hs00987206_m1 for B7-H3 and HS02758991_G1 for GAPDH.

### 4. Measurement of Apoptosis in AML Cell Lines and Primary Cells

For live-cell apoptosis analysis, the inventors stained OCI-AML3, MV4-11, U937, and primary cells with CytoLight Red (#4482; Essen BioScience). The cells were incubated with annexin V green reagent simultaneously with different concentrations of anti-B7-H3 mAbs (T-1A5, HEK5-1B3, and 58B1) along with IgG1 control and a chimeric T-1A5 antibody (ChT-1A5, described below) with rituximab in the presence or absence of different ratios of NK cells (effector: target). Time-lapse fluorescence imaging was performed using an Incucyte live-cell imaging system (Essen BioScience) to assess apoptosis induction every 1 hour for 20-48 hours. The apoptosis in leukemic cells was measured by green and red overlap counts.

5. Chimeric Antibody Generation from mAb T-1A5

Sequencing of T-1A5 antibody variable heavy (VH) and variable light (VL) chains were performed at Absolute Antibody. The resulting antibody sequences were cloned in a standard cloning vector. Antibody sequences were verified by DNA sequencing at the Advanced Technology Genomics Core, MD Anderson Cancer Center. Next, the VH and VL fragments were subcloned into pFUSEss vectors (InvivoGen), which contain the constant heavy chain sequences of the human IgG. The final clone was sequenced again to confirm the antibody sequence and its orientation.

### 6. Antibody Purification

Expi293 cells (Expi293 Expression System, Thermo Fisher Scientific) were co-transfected with a recombinant pair (pFHC1/pFLC1) to produce the IgG antibody clone 1. After transfection, the inventors collected the supernatant and tested binding of the antibody to its target by enzyme-linked immunosorbent assay (ELISA) as previously described (34-36). Briefly, a 96-well microplate was coated with 0.1 μg/mL of antigen in PBS (50 μL/well). Plates were incubated overnight at room temperature in a moisture chamber. Blocking was performed with 2% PBST (PBS plus 0.1% Tween-20 and 5% bovine serum albumin) at room temperature for 1 hour. After washing 3 times with PBST, 50 μL/well of supernatant containing chimeric antibody was added and incubated for 1 hour at room temperature. Secondary antibody goat anti-human IgG conjugated to horseradish peroxidase (Jackson ImmunoResearch) was added following the manufacturer's recommendations (50 μL/well, 1:2000) and incubated for 1 hour at room temperature. Color was developed using TMB substrate solution. The reaction was stopped with 0.2 M H₂SO₄, and the color reaction was read at OD480 nm.

The chimeric antibody was purified from supernatant of the recombinant Expi293 cells. Briefly, 1 L of supernatant containing the antibody was adjusted to pH 8-9 with 1 M Tris-HCl (pH=9; Teknova). Then, it was loaded to MabSelect SuRe resin column (GE Healthcare), washed once with PBS, and eluted with 0.1 M glycine plus 0.1 M CaCl₂ (pH=3). Fractions of eluent, 1 mL each, were collected and neutralized in tubes containing 50 μL of 1 M Tris HCl. Antibody concentration in each fraction was determined at OD280 nm. Pooled positive fractions were dialyzed (MW=6000-8000 Da; cutoff; Fisher) and concentrated (Amicon Ultra-4 10K) (36). Validation and quality control tests of the purified antibody for specificity (binding screening by ELISA), purity (Ultra High Performance Liquid Chromatography; UHPLC), and endotoxin (Lonza Endotoxin kit) were conducted following recommendations for rigor and reproducibility by the International Working Group for Antibody Validation (37).

### 7. Pharmacokinetic Analysis of the Anti-B7-H3 Antibodies

The Octet RED384 System (FortdBio) was used to further characterize the chimeric and murine clones. The kinetics of the clones were tested by measuring the interaction between the antibody and its target by bio-layer interferometry technology. The system measured pharmacokinetics (affinity, avidity, association constant, and dissociation constant (Kd), and the affinity constant of each antibody was calculated using the Data Acquisition Software (36). The antibodies were immobilized using the corresponding biosensors (AMC #18-5088 or AHC #18-5060) and observed interacting with their antigen added in a soluble format following the manufacturer's recommendations (38).

### 8. In Vivo Mouse Studies

a. Using Anti-B7-H3 Antibodies Alone

Female 6- to 8-week-old NSG mice (Il2rg^{null}; n=50) were purchased from The Jackson Laboratory. All the mice were housed in the Animal Core Facility (Houston) at MD Anderson Cancer Center. All mouse experiments were performed in accordance with MD Anderson Institutional Animal Care and Use Committee (IACUC) guidelines and were approved by the committee. Patient-derived AML blasts were obtained from sample 882-A and expanded in vivo in NSG mice. AML PDX cells (2×10⁶) were injected into 6- to 8-week-old female NSG mice through tail vein injections, and engraftment was monitored weekly via flow cytometry analysis of peripheral blood (PB) CD45-positive cells. When the majority of mice had >1% AML blasts in PB, they were randomized and injected intraperitoneally with anti-B7-H3 antibodies (T-1A5, HEK5-1B3, and 58B1) or IgG1 isotype antibody as a control at a dose of 1 mg/kg twice a week. Mice were monitored and euthanized according to MD Anderson IACUC-approved protocols.

b. Anti-B7-H3 Antibodies in Combination with NK Cells in AML Xenograft and PDX Models

For mouse xenograft models, AML cell lines (OCI-AML3) were transduced with a retroviral vector encoding the enhanced green fluorescent protein (eGFP) firefly luciferase (eGFP-FFluc) gene (39, 40). A single cell clone was first selected based on high eGFP expression and in vitro FFluc activity. eGFP-FFluc-OCI-AML3 cells (2×10⁶) were injected into the lateral tail vein of 6- to 8-week-old female NSG mice (Il2rg^{null}; n=30). Engraftment was monitored via bioluminescence imaging (BLI) using an IVIS Lumina II in vivo imaging system (PerkinElmer). After engraftment, mice were randomized into three treatment groups: 1) control (PBS), 2) NK cells plus mouse IgG1, and 3) NK cells plus mAb T-1A5. Human NK cells (10×10⁶ cells/mouse) were given intravenously twice a week in combination with IgG1 or anti-B7-H3-T-1A5 mAbs (once a week by intraperitoneal injection) at 1 mg/kg for 7 weeks. Leukemia growth was monitored via BLI using the IVIS Lumina II system (PerkinElmer). A similar experiment was performed using ChT-1A5 in combination with human NK cells in a B7-H3-positive AML PDX model. The patient-derived AML blasts were obtained from sample 452-A and expanded in vivo in NSG mice.

### 9. Molecular Modeling of B7-H3 and Antibody Fragments

Owing to the lack of crystal structure for human B7-H3, the inventors used a homology modeling approach to generate the 3D model for human B7-H3 based on murine B7-H3 (Protein Data Bank: 4I0K) as a template. The protein sequence alignment of human B7-H3 isoform 2 showed 88.61% sequence identity with murine B7-H3. Homology modeling panel in the Prime module available in Maestro software (Schrödinger, Inc.) was used to build the B7-H3 model and antibody fragments (VH and VL) of the antibody. Protein docking was performed by assigning the B7-H3 model as receptor and antibody fragments as ligands in the Protein-Protein Docking panel. The docked complex with the best docking score was selected, and pose representations were generated in the Maestro visualizer or PyMOL software. Among the docking poses, the complexes of complementary determining region elements in T-1A5 VH or VL interacting with the 2Ig domain of B7-H3 were manually selected.

### 10. Statistical Analysis

Patient characteristics were extracted from the MD Anderson Cancer Center EPIC (Epic Systems Corporation) electronic health records. B7-H3 expression was evaluated in a total of 100 AML samples and 20 normal controls. Patients were categorized into three different prognostic risk groups, i.e., good, intermediate, and poor risk, based on the European LeukemiaNet (ELN) classification (41). Expression of B7-H3 was reported as mean±standard deviation across all categorical variables. Mean B7-H3 expression was compared between control and AML samples using the Wilcoxon rank-sum test. The inventors performed various statistical analysis in Oregon Health & Science University (OHSU) AML dataset. B7-H3 expression was then categorized into a binary variable. A receiver operating characteristic curve was used to choose an appropriate cutoff value of MFI=2894.5. Overall survival was calculated from the date of diagnosis to death from any cause. Disease-free survival was determined from the date of diagnosis to the date of relapse or last follow-up. The censored date for alive patients was Mar. 1, 2021. A log-rank test was done to generate Kaplan-Meier overall survival curves based on B7-H3 values. All patient data analyses were performed on the IBM SPSS Statistics software for Windows v26.

The statistical significance of tumor growth was determined using a 2-way analysis of variance for repeated measures. All other group differences were evaluated using a 2-tailed unpaired Student t-test. All figures and analyses were generated using Prism 8 (GraphPad Software). All figures and analyses for flow cytometry were generated using FlowJo software. p values<0.05 were considered significant.

### C. Results

1. B7-H3 Expression in AML and its Association with Clinical Outcomes

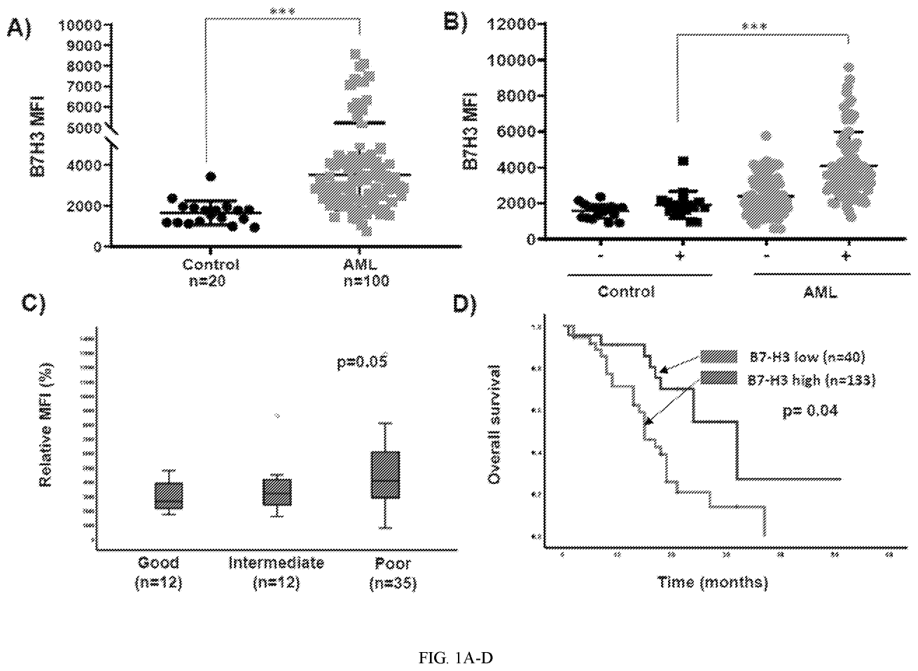

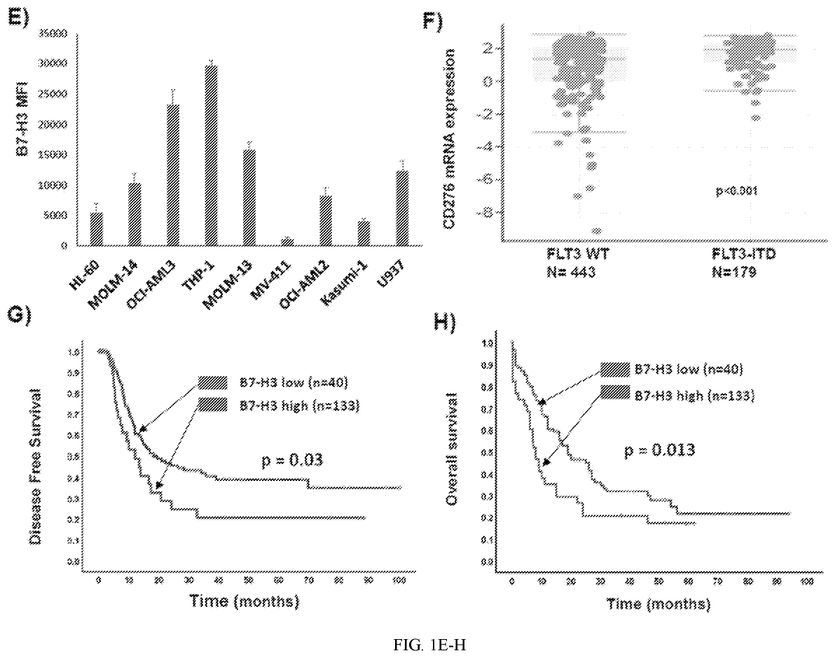

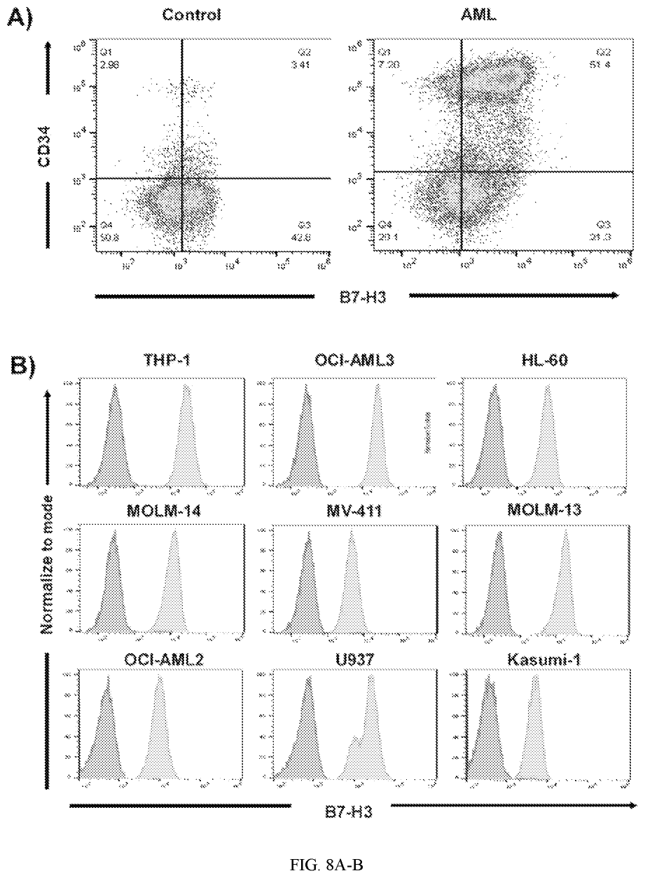

The inventors analyzed B7-H3 expression in PB and bone marrow mononuclear cells from AML patients and healthy donors at MD Anderson Cancer Center. The baseline characteristics of these patients are tabulated in Suppl. Table 1. Cell surface expression analysis by flow cytometry revealed that the cells of ˜60% of AML patients were positive for B7-H3, and its expression was 2- to 3-fold higher in AML patients than in healthy donor counterparts (p<0.01) (FIG. 1A). Next, the inventors measured B7-H3 expression in progenitor cells from healthy individuals and AML patients. In AML patients, B7-H3 expression was significantly higher in CD34-positive cells than in the CD34-negative subpopulation (p<0.001). In contrast, there was no difference in B7-H3 expression between CD34-positive and CD34-negative cells from healthy donors, indicating that B7-H3 is strongly expressed in AML blast cells (p<0.001) (FIG. 1B, FIG. 8A). To examine the clinical and prognostic significance of B7-H3 expression in AML, the inventors stratified patients into favorable, intermediate, and poor risk groups based on their European LeukemiaNet prognostic risk score (41). B7-H3 expression was significantly higher in patients with poor prognostic scores (p=0.05) (FIG. 1C). Additionally, the inventors stratified patients into low and high B7-H3 expression groups based on mean B7-H3 expression values as described in the Methods. Patients with high B7-H3 expression had significantly worse overall survival than those with low B7-H3 expression (p=0.04) (FIG. 1D). Furthermore, the inventors observed varied degrees of B7-H3 expression in 9 different AML cell lines (FIG. 1E, FIG. 8B).

These findings regarding B7-H3 overexpression in AML patients were supported by mRNA expression analysis performed on the OHSU AML dataset, available in the public domain (42). Moreover, patients with FLT3-ITD mutations had higher B7-H3 mRNA expression compared to patients with wild-type FLT3 (p<0.001) (FIG. 1F). Additionally, B7-H3 mRNA overexpression correlated with significantly worse disease-free and overall survival (p=0.013) (FIG. 1G, H). These results indicate that B7-H3 is upregulated in AML and that its expression is strongly correlated with unfavorable clinical outcomes, rendering it an important therapeutic target with potential clinical benefits.

### 2. Inhibition of B7-H3 Induced NK Cell-Mediated Killing in AML Cells

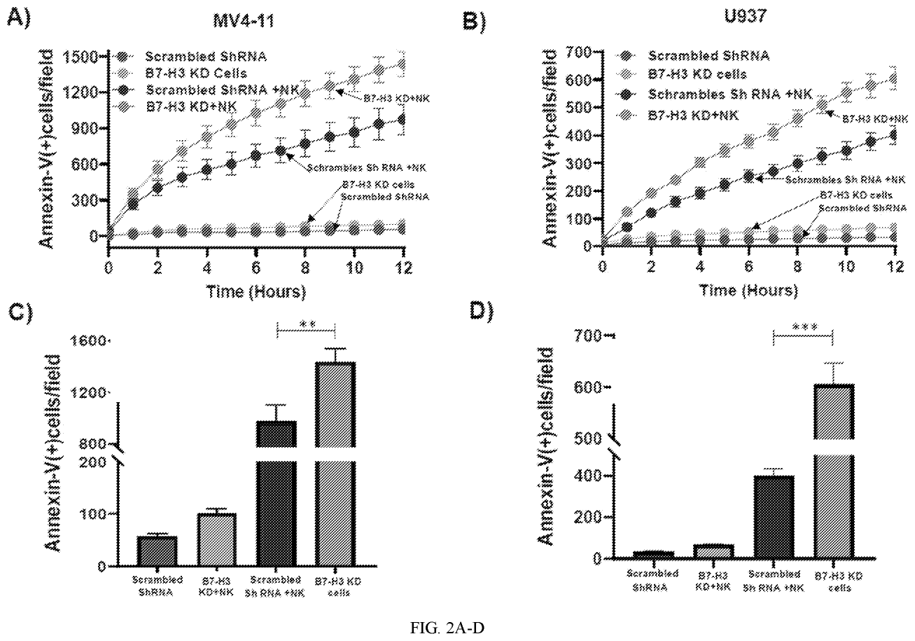

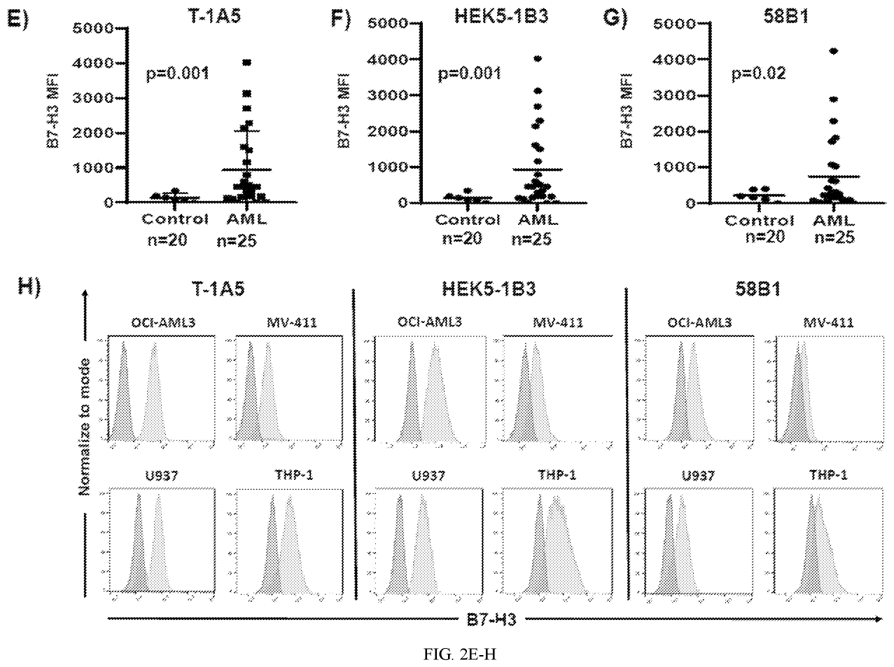

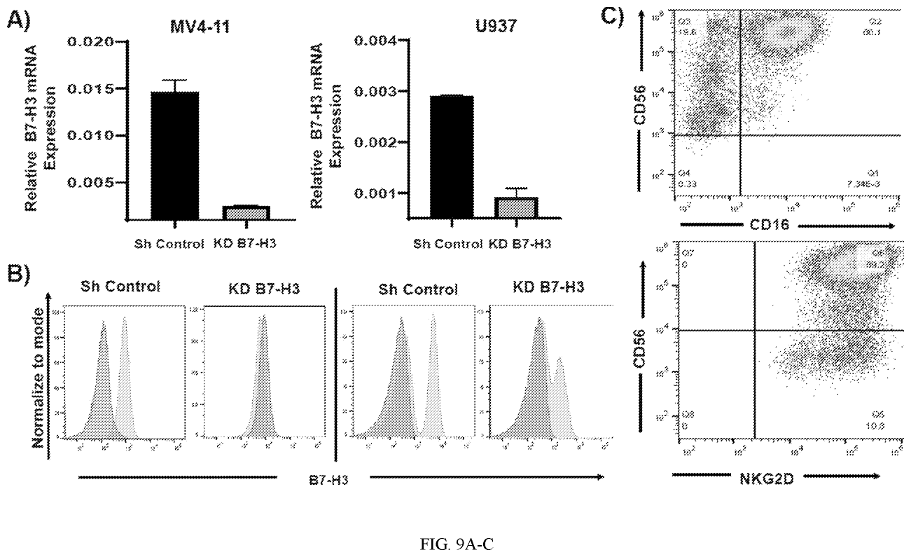

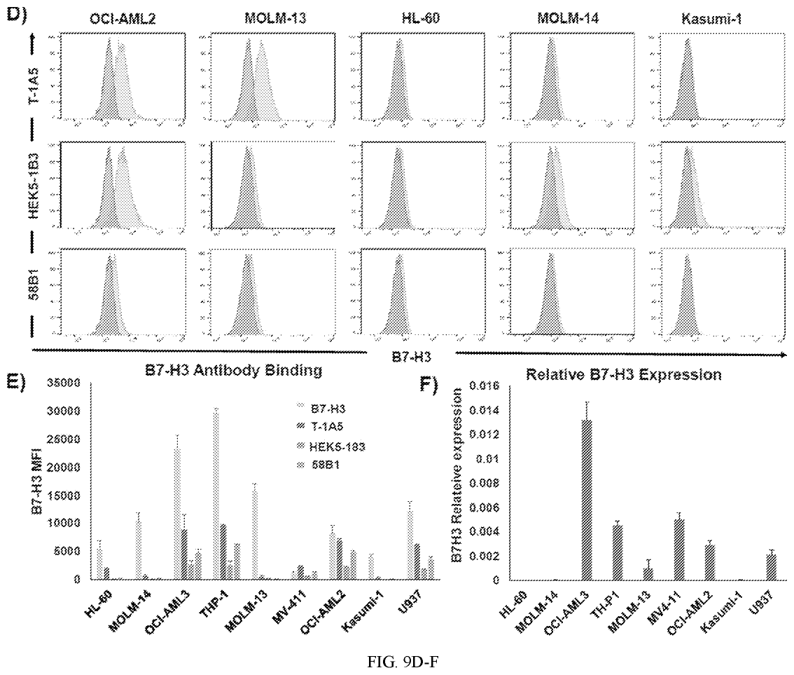

To investigate the role of B7-H3 in altering the AML immune microenvironment, the inventors performed stable lentiviral knockdown of B7-H3 in MV4-11 and U937 AML cell lines, which had high B7-H3 expression. The inventors then validated the knockdown efficiency at mRNA and protein levels by quantitative RT-PCR and flow cytometry, respectively, and found up to 70% inhibition of B7-H3 expression in knockdown cells compared to scrambled shRNA-treated control cells (FIG. 9A, B). Further, the inventors analyzed the activity of NK cells and observed expression of activation markers CD16 and NKG2D in more than 80% CD56-positive cells (FIG. 9C). Next, the inventors co-cultured B7-H3 knockdown cells in the presence and absence of NK cells in a 2:1 (effector: target) ratio and measured the induction of apoptosis in AML cell lines (MV4-11 and U937) through annexin V binding using Incucyte live-cell imaging for 12 hours. The inventors found a time-dependent increase in NK cell-mediated apoptosis in all the B7-H3 knockdown AML cell lines compared to control shRNA-treated cells (FIG. 2A, C). Quantitative analysis at the 12-hour timepoint also revealed a 3-fold increase in NK cell-mediated killing in B7-H3 knockdown AML cell lines (p<0.01) (FIG. 2B, D). Taken together, these results suggest that the depletion of B7-H3 leads to increased NK cell-mediated apoptosis in AML cells.

### 3. Anti-B7-H3 Antibodies Enhanced NK Cell-Mediated Apoptosis in AML Cell Lines

To block B7-H3's immunomodulatory function, the inventors tested three novel anti-B7-H3 mAbs (T-1A5, HEK5-1B3, and 58B1 (43), and measured their binding affinities using flow cytometry in AML patients and cell lines. As expected, B7-H3 expression was significantly higher in AML patients compared to the control group for all 3 antibodies tested (p<0.02) (FIG. 2E-G). The inventors also noted that the anti-B7-H3 antibodies had strong binding to the OCI-AML3, U937, MV4-11, and ThP-1 cell lines (FIG. 2H) in contrast to MOLM-13, MOLM-14, HL-60, and Kasumi-1 (FIG. 9D, E). Further, the inventors measured B7-H3 mRNA expression by quantitative RT-PCR and observed a strong overlap between B7-H3 mRNA and protein expression (FIG. 9F), suggesting that these antibodies are highly specific to B7-H3.

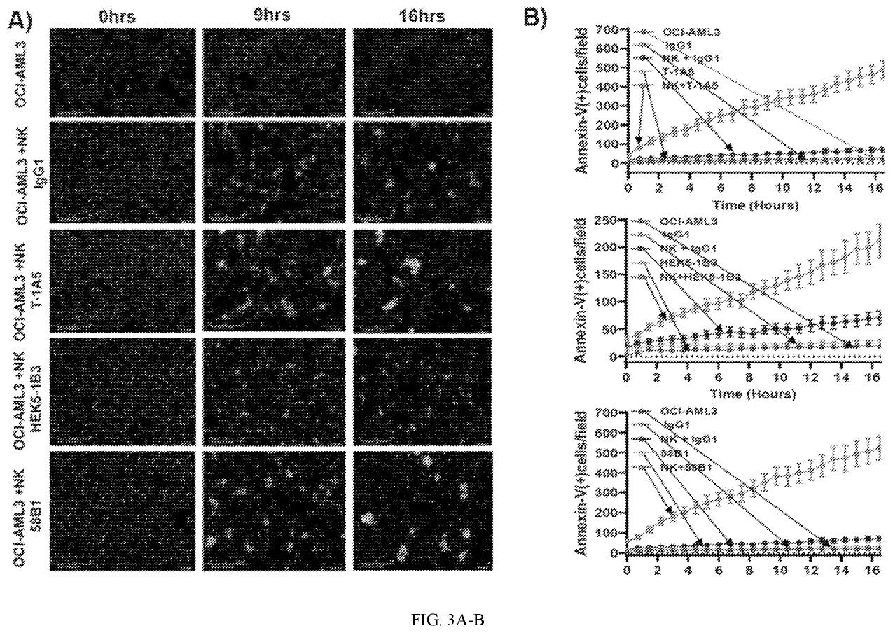

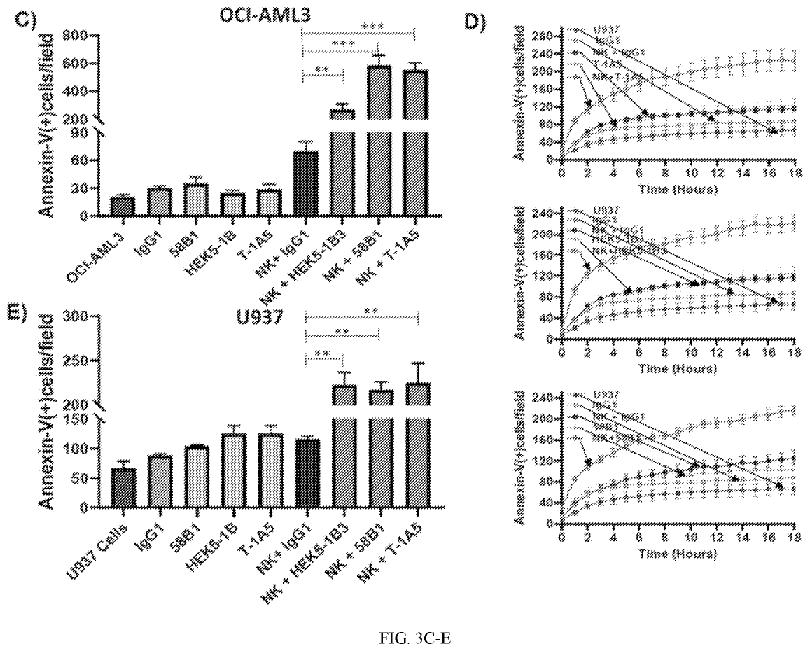

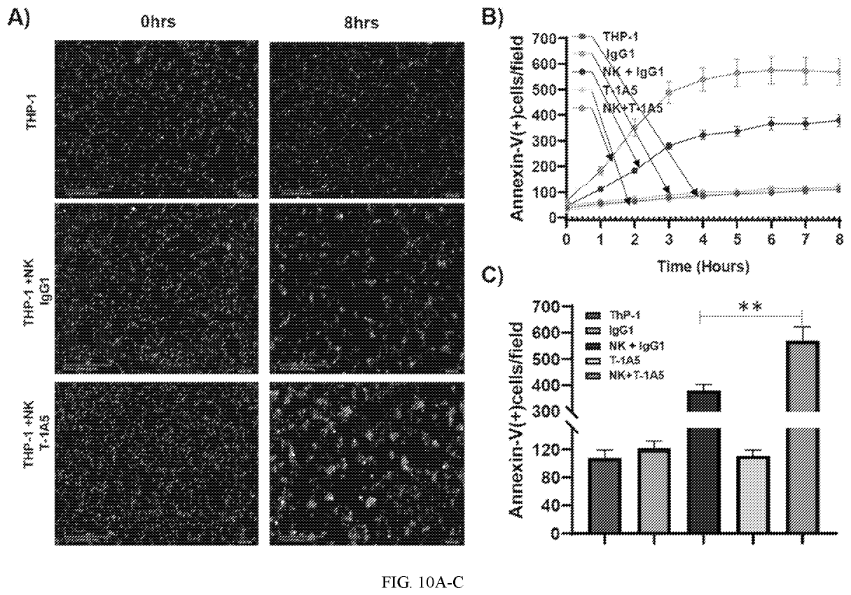

To determine the effect of anti-B7-H3 antibodies in blocking the immunomodulatory functions of B7-H3, the inventors co-cultured AML cell lines (OCI-AML3 and U937) with and without NK cells, in the presence or absence of anti-B7-H3 antibodies including T-1A5, HEK5-1B3, and 58B1 (1 μg/mL) in a 2:1 NK to target cell ratio, and the inventors measured apoptosis an using annexin V binding assay by the Incucyte live-cell imaging system. Representative images of AML cells undergoing apoptosis at various time points after treatment with anti-B7-H3 antibodies and NK cells are illustrated in FIG. 3A. In contrast to IgG1 control antibody treatment, anti-B7-H3 antibodies in combination with activated NK cells showed a significant increase in annexin V binding within a few hours. Contrastingly, IgG1 control or anti-B7-H3 antibody treatment alone did not induce cell death in these cells (p<0.001) (FIG. 3B-E). Further, the inventors validated these findings in the ThP-1 AML cell line, which has high B7-H3 expression as well. As expected, the inventors observed that the combination of anti-B7-H3 antibodies with activated NK cells significantly increased annexin V binding on THP-1 cells (p<0.001) (FIG. 10A-C). Overall, these results indicate that anti-B7-H3 antibodies block the immunomodulatory functions of B7-H3 and enhance NK cell-mediated apoptosis in AML.

### 4. Anti-B7-H3 Antibodies Inhibit Leukemia Growth and Extend Survival in Animal Models

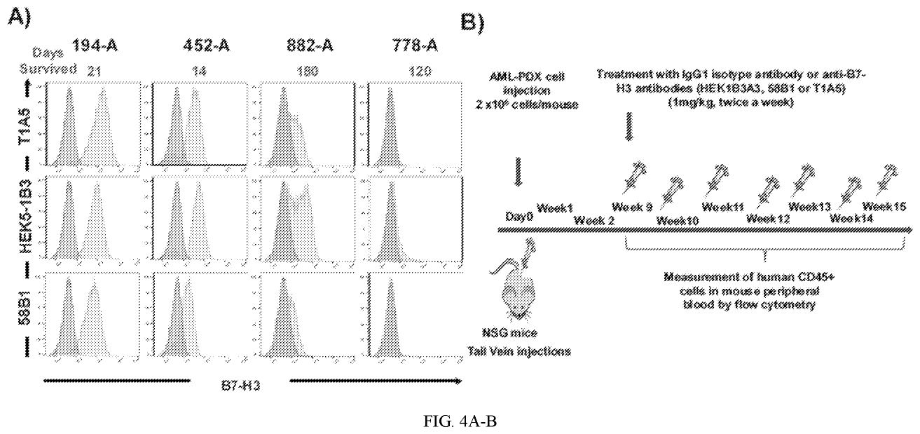

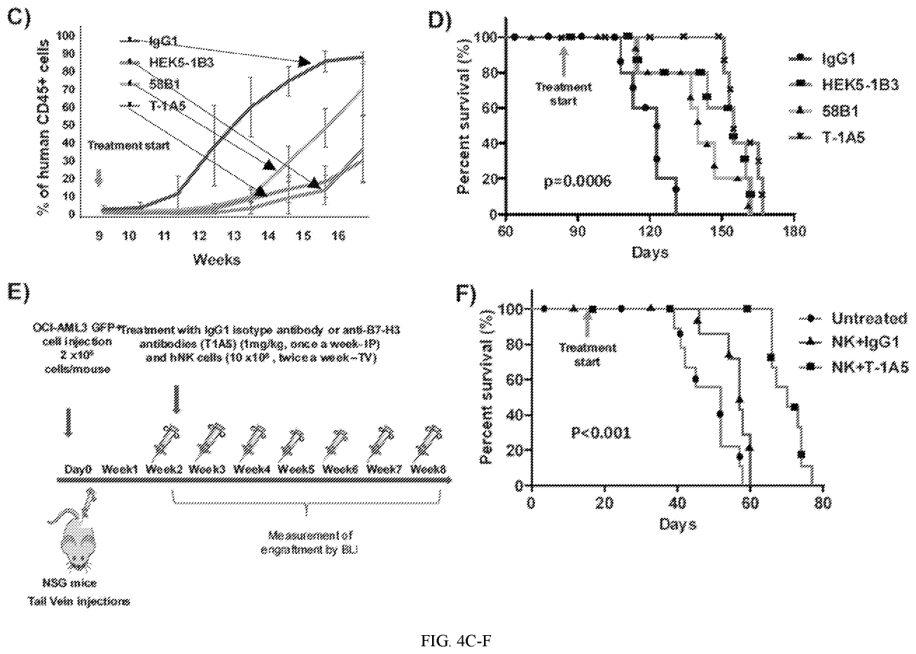

To study the effect of B7-H3 expression on leukemia growth, 4 different AML PDX models were developed. The clinical parameters are listed in Suppl. Table 2. The inventors observed variable expression of B7-H3 across the PDX models, and the models with high B7-H3 expression were found to be more aggressive (FIG. 4A). To determine which clone of the anti-B7-H3 antibodies is most effective against primary human AML blasts, the inventors treated AML PDX cells developed in the inventors' laboratory with all 3 anti-B7-H3 antibodies mentioned above. Nine weeks after injection, when the percentage of AML blasts in PB reached >1%, mice were intraperitoneally injected with IgG1 isotype antibody or anti-B7-H3 antibodies at 1 mg/kg/day twice a week for 15 weeks (FIG. 4B). AML growth was determined on a weekly basis by measuring human CD45-positive cells in PB of mice using flow cytometry. The inventors observed a significant decrease in AML growth in mice treated with anti-B7-H3 antibodies compared to mice treated with IgG1 antibody (FIG. 4C). In addition, the inventors found that anti-B7-H3 antibody treatment significantly improved overall survival of mice compared to control treatment (p<0.0001) (FIG. 4D). Moreover, antibody generated from T-1A5 clone provided better survival advantage compared to other two antibodies.

Next, to study the cumulative effect of anti-B7-H3 mAbs and NK cells in xenograft models, the inventors implanted OCI-AML3 cells expressing firefly luciferase (2×10⁶ cells/mouse) in NSG mice via tail vein injections. Seven days later, when the BLI of tumors reached 1.5×10⁹ photons/second, mice were treated IP with human NK cells (10×10⁶ cells/mouse) twice a week in combination with single treatment per week of IgG1 or an anti-B7-H3 mAb (T-1A5) at 1 mg/kg (FIG. 4E). Moreover, Kaplan-Meier survival analysis revealed that mice treated with NK cells in combination with T-1A5 had a significant survival advantage compared to mice left untreated or given NK cells in combination with IgG (p<0.001) (FIG. 4F). These results indicate that B7-H3 blockade using T-1A5 antibody enhances NK cell-mediated cytotoxicity in AML cells and extends the survival of mice bearing leukemia.

### 5. Characterization of Chimeric Anti-B7-H3 Antibody (ChT-1A5)

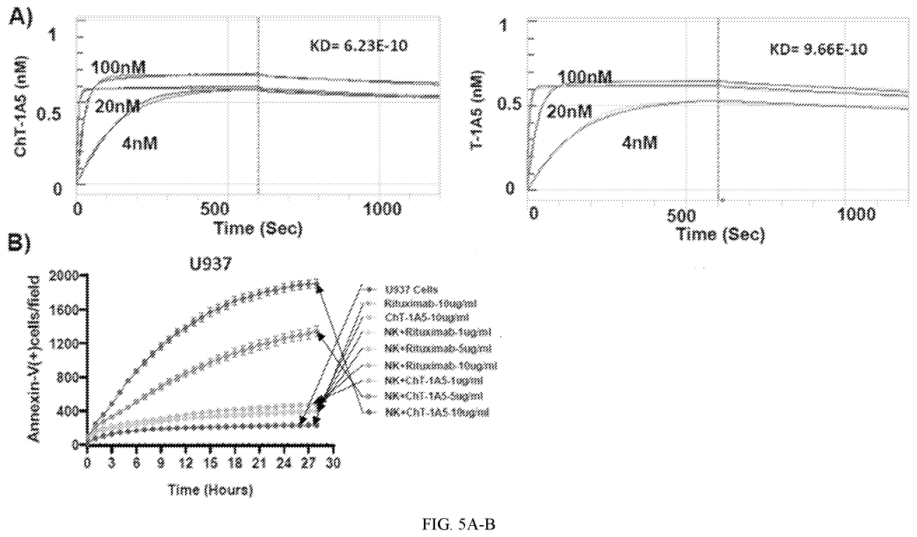

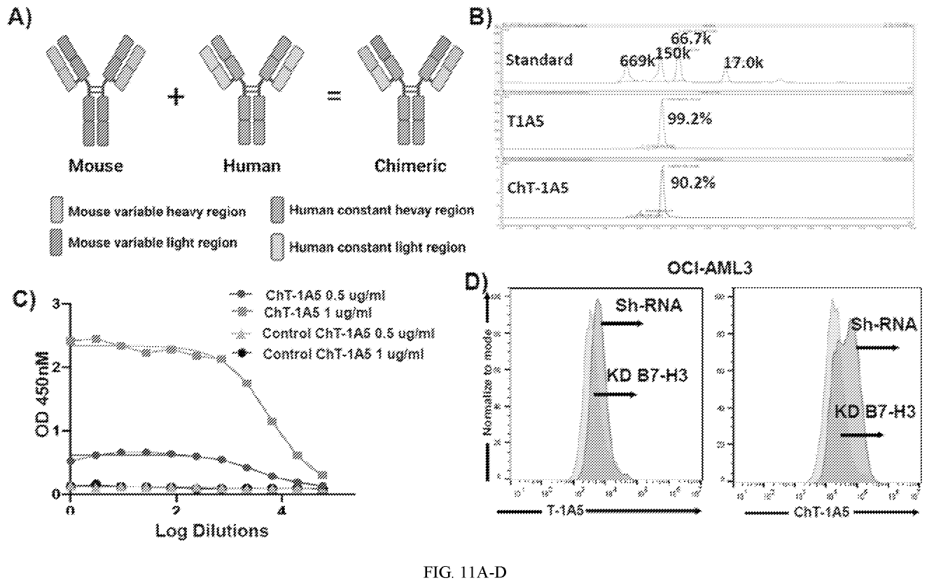

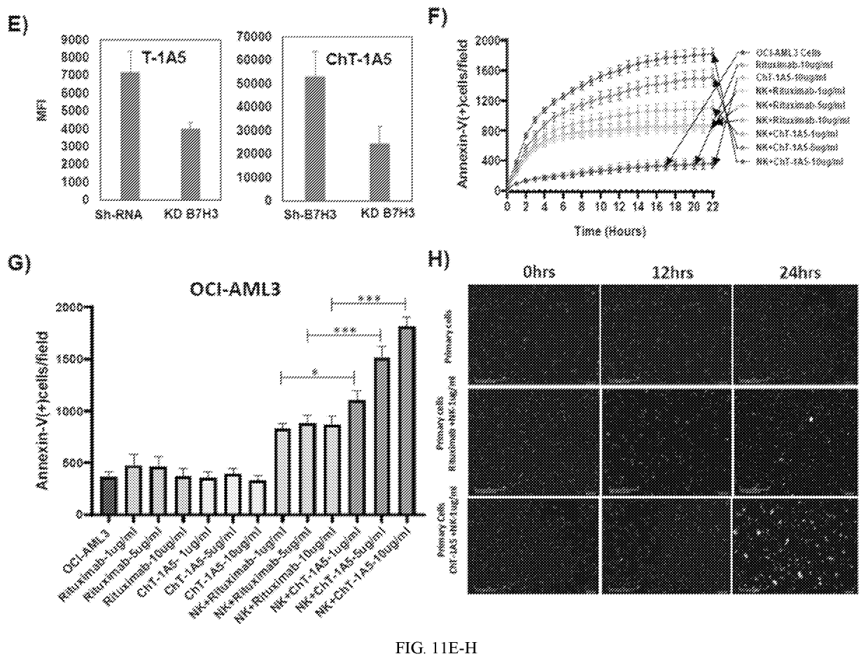

To develop an antibody for therapy of B7-H3-positive AML, the inventors generated a human-mouse chimeric antibody (FIG. 11A) based on the VL and VH antibody sequences derived from the clone T-1A5 (ChT-1A5) and measured its purity using UPHLC. The results suggest that ChT-1A5 antibody has purity of >98% (FIG. 11B). Further, the inventors measured its binding efficiency using ELISA and found strong binding to B7-H3 protein (FIG. 11C). Additionally, the inventors validated the sensitivity of ChT-1A5 and mouse T-1A5 mAbs using flow cytometry in B7-H3 knock down OCI-AML3 cells. Mean fluorescent intensity of ChT-1A5 (MFI-20,000) was observed to be fivefold higher than anti-B7-H3-T-1A5 antibody (MFI-4000) (FIG. 4D-E). To assess the binding affinity of the antibodies, pharmacodynamics studies were performed using the Octet RED384 system. The inventors found strong binding of both ChT-1A5 and T-1A5 antibodies to the recombinant human B7-H3 protein, with dissociation constants (Kd) of 6.23×10⁻¹⁰ M and 9.66×10⁻¹⁰ M for ChT-1A5 and mouse T-1A5 antibodies, respectively (FIG. 5A).

### 6. ChT-1A5 Induced NK Cell-Mediated Antibody-Dependent Cellular Cytotoxicity in AML Cell Lines and Primary Cells

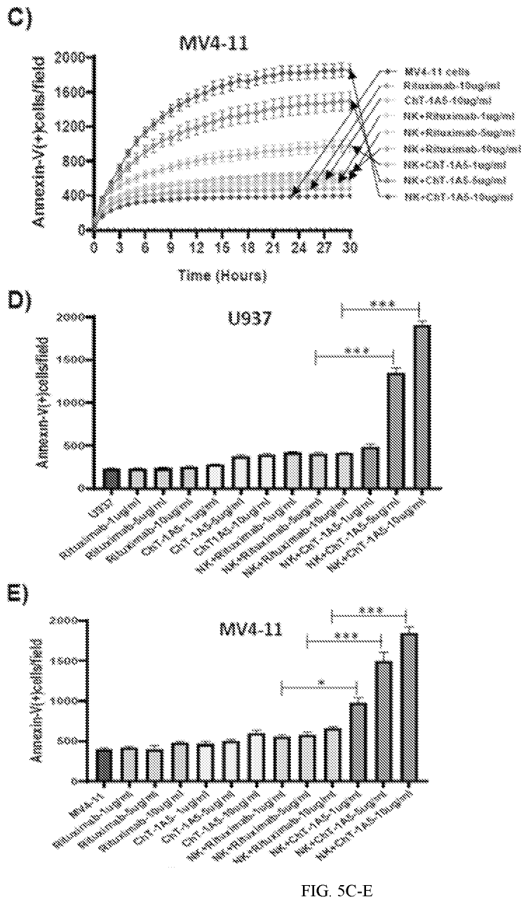

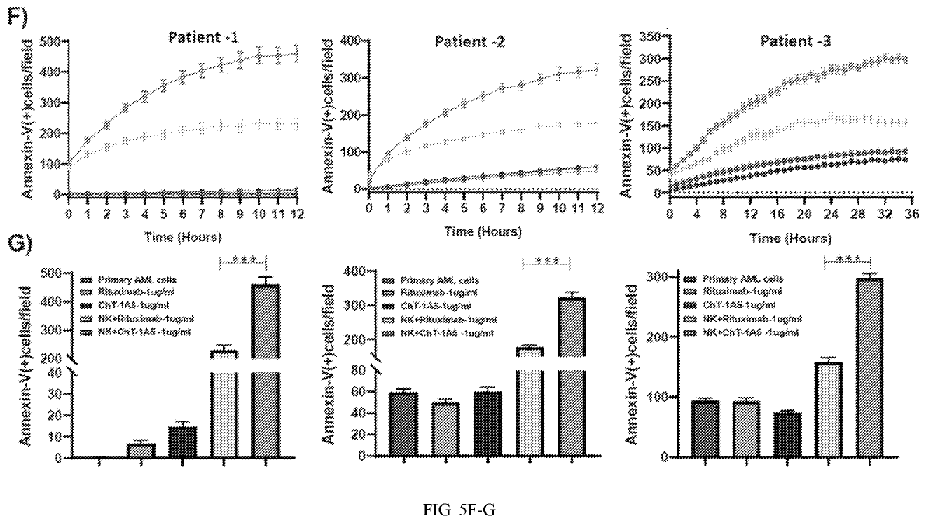

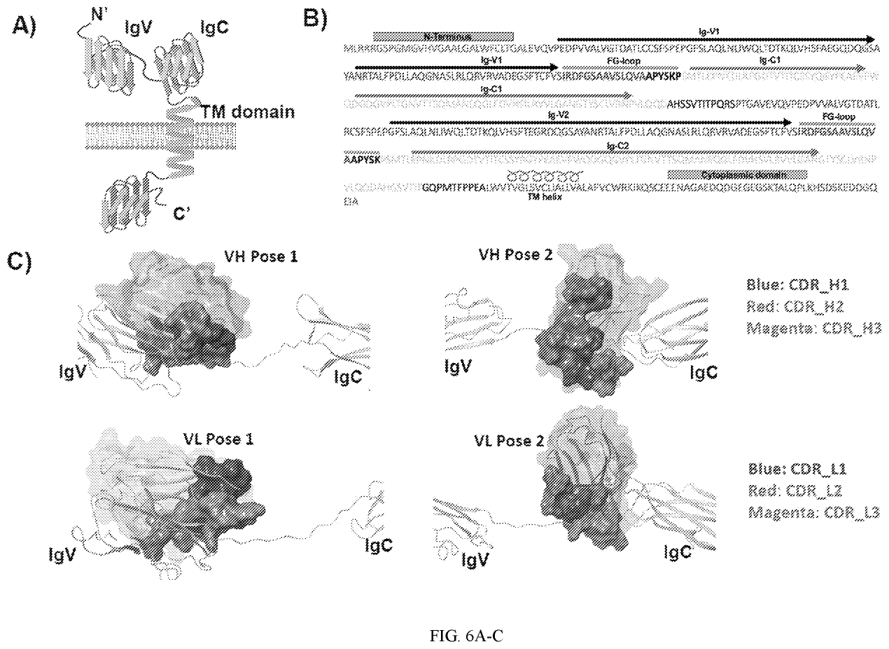

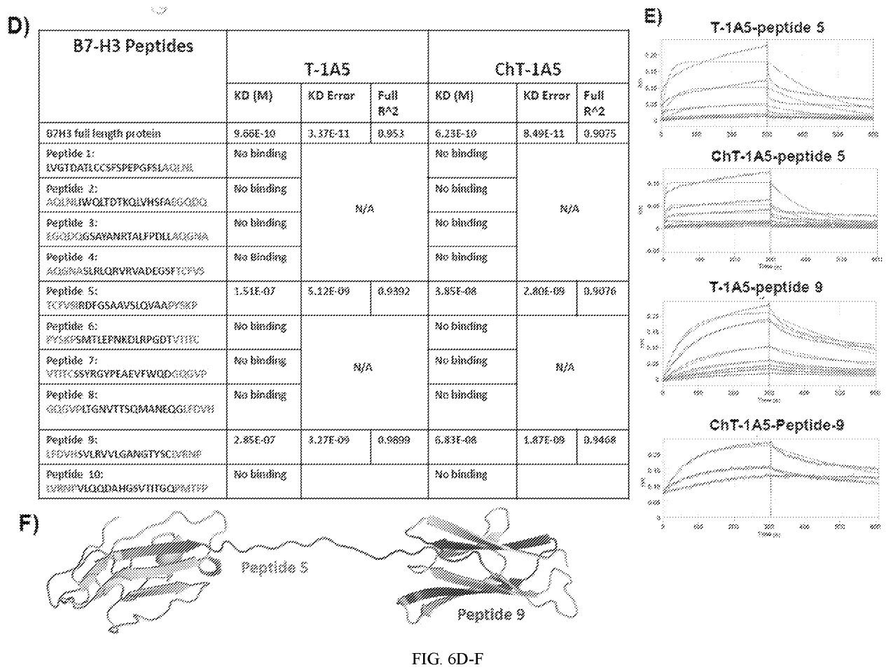

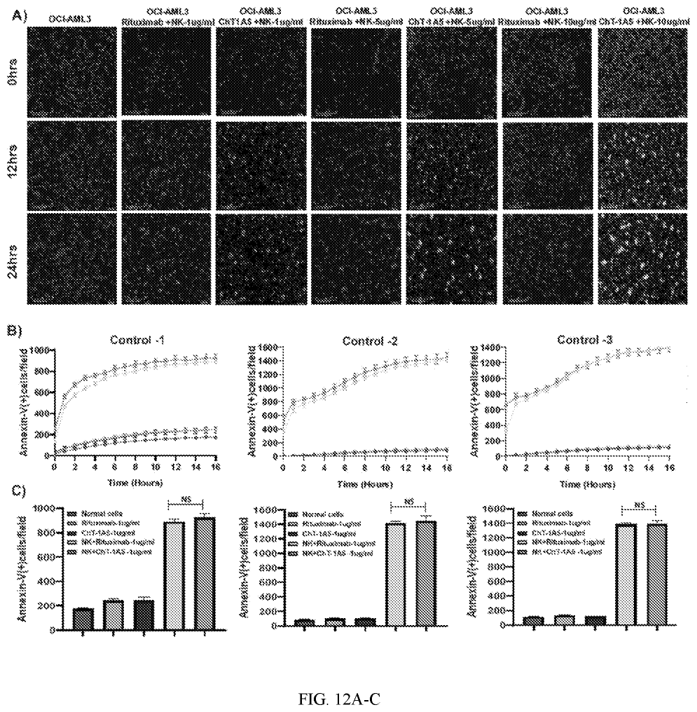

Since ChT-1A5 has a human Fc (fragment crystallizable) region, the inventors hypothesized that ChT-1A5 induces ADCC in B7-H3-positive AML cells. The inventors treated AML cell lines (OCI-AML3, MV4-11, and U937) with ChT-1A5 or rituximab (1, 5, and 10 μg/mL) in the presence or absence of NK cells at a 8:1 NK to target cell ratio. Rituximab is a chimeric anti-CD20 antibody that was used as a non-specific control antibody. NK cell-induced apoptosis was measured by annexin V binding assay using the Incucyte live-cell imaging system. The inventors found a dose-dependent increase in apoptosis of leukemic cells treated with ChT-1A5 antibody and NK cells, while rituximab with NK cells did not show significant outcomes (FIG. 5B, C FIG. 11F). As shown in FIG. 6D, E FIG. 11G, at time point 28 (MV4-11 and U937) and 22 (OCI-AML3) hours, treatment with ChT-1A5 or rituximab alone or in combination with NK cells had no effect on killing leukemic cells, whereas ChT-1A5 with activated NK-cells showed significant apoptosis in all 3 AML cell lines. Representative images of OCI-AML3 cells undergoing apoptosis at different time points in different treatment conditions are shown in FIG. 12A. Next to determine the effect of ChT-1A5 on primary AML cells, the inventors treated AML patient-derived PBMCs with ChT-1A5 plus NK cells or rituximab plus NK cells. The inventors found that the combination of chT-1A5 and NK cells induced significantly higher apoptosis in leukemic cells compared to rituximab plus NK, in a time-dependent manner (FIG. 4F, G; FIG. 11H). In contrast, the inventors did not find any such difference in PB cells derived from healthy donors treated with ChT-1A5 plus NK cells or rituximab plus NK cells (FIG. 12B, C). These findings suggest that ChT-1A5 antibody induces ADCC in both primary AML cells as well as cell lines expressing B7-H3, but not in cells derived from healthy donors.

### 7. Monoclonal Antibodies T-1A5 and ChT-1A5 Bind to the FG Loop Region of B7-H3

A recent study found that the FG loop region on the mouse B7-H3 protein is responsible for B7-H3's immunomodulatory function. Since T-1A5 is able to block B7-H3, the inventors hypothesized that T-1A5 binds to the FG loop region of the human B7-H3 protein. To identify the antibody binding sites for the T-1A5 antibody on the B7-H3 protein, the inventors marked the already reported structural elements of human B7-H3 (FIG. 6A, B). In humans, the extracellular domain of B7-H3 comprises 2 isoforms, i.e., 2Ig and 4Ig. For ease of studying, the inventors used a homology modeling approach and generated a 3D model of 2Ig using the crystal structure of murine B7-H3 as a template that shares 88.61% of its sequence identity. Next, the predicted models of VH or VL domains of T-1A5 antibody were docked (FIG. 6C). The 2 best poses of VH and VL docked on either IgV or IgC domains of B7-H3 were further analyzed. Interestingly, pose 1 of VH or VL was docked at the intersection of IgV and the FG loop, while pose 2 represented the binding towards the FG loop intersection with IgC. These predicted interactions indicated binding of the T-1A5 antibody to specific domains of B7-H3.

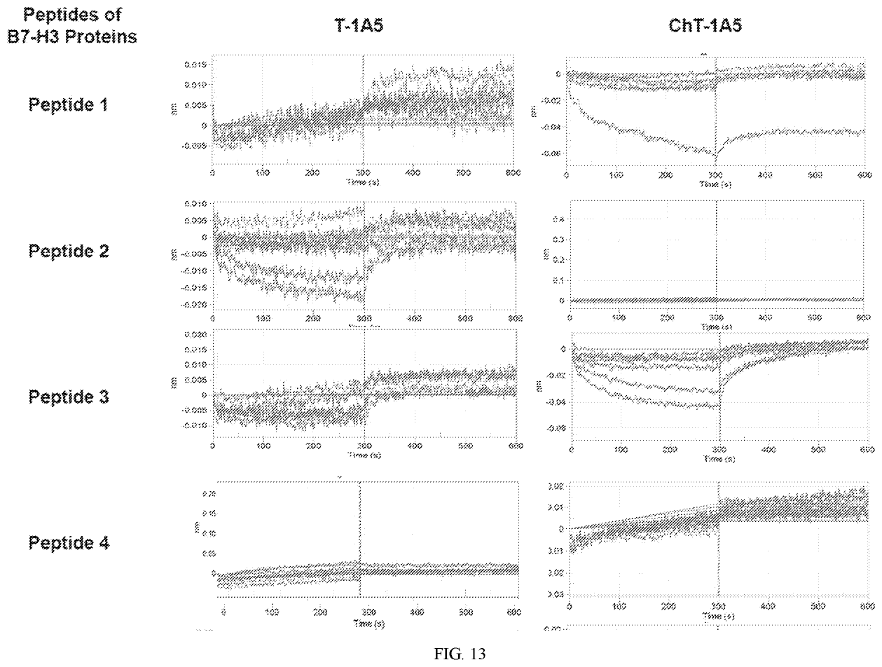

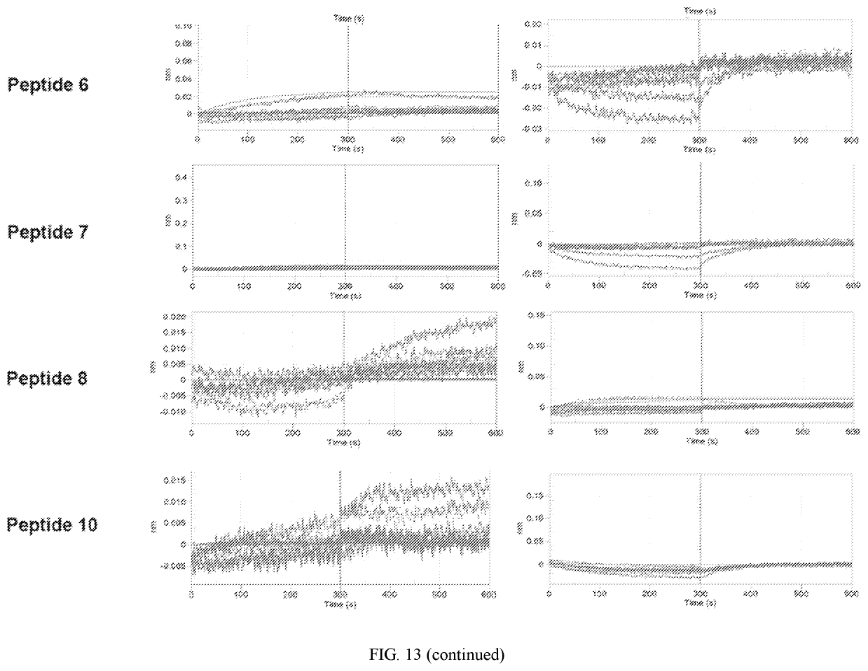

To validate these findings, epitope mapping was performed using peptide fragments derived from human B7-H3 protein. The inventors generated 10 peptides from the extracellular domain of B7-H3 with a span of 25 amino acids and an overlap of 5 amino acids with each subsequent peptide. Interestingly, a biolayer interferometry assay using the Octet system showed that peptide 5 and peptide 9 had stronger binding affinity with both the mouse antibody (T-1A5) and the chimeric antibody (ChT1A5; FIG. 6D, E), while the other peptide did not bind to either antibody (FIG. 13). Peptide 5 showed Kd values of 3.04×10⁻⁷ and 3.85×10⁻⁸ with T-1A5 and chT-1A5 respectively, while peptide 9 showed Kd values of 2.85×10⁻⁷ and 6.83×10⁻⁸ with T-1A5 and ChT-1A5, respectively. It is plausible that peptides show lower affinity than the B7-H3 full protein, with KD values 9.66×10⁻¹⁰ and 6.23×10⁻¹⁰ with T-1A5 and ChT-1A5, respectively. However, both peptide 5 and peptide 9 had higher affinity towards ChT-1A5 compared to T-1A5, which is also evident from the Kd of the full B7-H3 protein. Strikingly, the inventors' further analysis of the B7-H3 model showed that peptide 5 (red) was present in the IgV domain spanning through the FG loop and that peptide 9 (blue) was present in IgC domain (FIG. 6F). Collectively, the results suggest that the structural conformation of both Ig domains linked with the FG loop of B7-H3 and that this is the binding site for T-1A5 or ChT-1A5 antibodies.

8. ChT-1A5 in Combination with Activated NK Cells Inhibits Leukemia Growth in B7-H3-Positive AML PDX Model

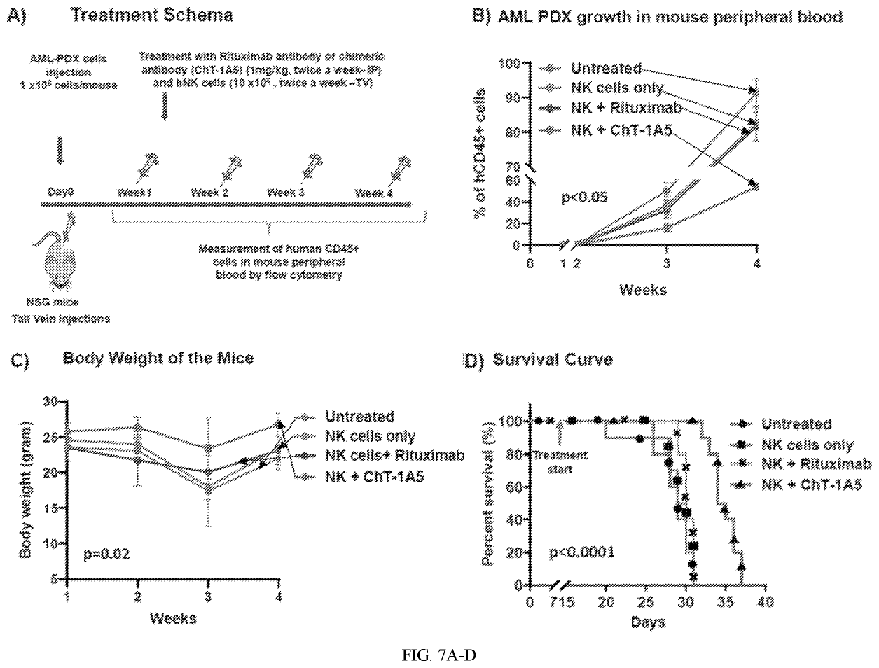

To evaluate the effect of the ChT-1A5 antibody in combination with activated NK cells, the inventors injected AML PDX cells (1×10⁶ cells/mouse) developed in the inventors' laboratory into NSG mice. One week after injection, when the percentage of AML blasts in PB reached >1%, the mice were randomly distributed in 4 groups: untreated, NK cells, NK cells plus ChT-1A5, and NK cells plus rituximab. The mice were treated intraperitoneally with chimeric anti-B7-H3 antibody (ChT-1A5) at 1 mg/kg/day in combination with NK (10×10⁶ cells/mouse; tail vein) twice a week for 4 weeks. (FIG. 7A). In all the experimental groups, AML growth was determined weekly by measuring human CD45-positive cells in PB of mice using flow cytometry. Mice treated with both ChT-1A5 and NK cells showed a significant delay in leukemia growth compared to the rest of the experimental mice groups (FIG. 7B). The overall weight of the untreated mice was significantly (p=0.02) reduced when compared to treated group (FIG. 7C). Also, Kaplan-Meier survival analysis revealed a considerable survival advantage of ChT-1A5 and NK cell-treated mice compared to the other groups (FIG. 7D). These findings demonstrated that chimeric ChT-1A5 antibody limits leukemia growth and extends survival of mice by directing NK cells to target B7-H3 through induction of ADCC. Thus, treatment with anti-B7-H3 antibody in combination with NK cells could be a potential immunotherapeutic approach for the treatment of AML.

### D. Discussion

In the new era of cancer treatment, the immunotherapy approach has gained momentum. Targeted immunotherapies against immune checkpoint inhibitors such as CTLA-4 and PD-L1 have shown promising survival outcomes in solid tumors (44, 45). However, this method of cancer treatment has been less explored with respect to hematological malignancies. Therefore, the inventors studied the immunomodulatory role of checkpoint molecule B7-H3 in AML. These results indicate that the B7-H3 molecule can serve as a new antibody-based immunotherapy target in AML.

The inventors found that ˜60% of patients with AML overexpressed B7-H3 in AML blast cells, which is associated with poor prognosis. This result compares well with the previously published reports of high B7-H3 expression and its association with poor clinical outcomes in AML patients (46, 47).

In this study, in vitro, the inventors observed that blockade of B7-H3 using mAbs alone inhibited leukemia growth in 1 of the PDX models (FIG. 4D). Considering that NSG mice lack most of the functional NK and T cells, the inventors suspect that residual monocytes or macrophages could induce ADCC or antibody-dependent cellular phagocytosis with the help of T-1A5 antibody. Moreover, in the xenograft mouse model, the combination of B7-H3 blockade with NK cells reduced leukemia growth and prolonged survival. In this experiment, it was clear that B7-H3 blockade augmented NK cell responses and inhibited leukemia growth. In solid tumors, it has been reported that blocking of B7-H3 using mAbs increases NK cell and CD8 positive T cell tumor infiltration and reduces cancer progression (10, 52, 53). These findings show for the first time that the anti-B7-H3 antibodies block immunomodulatory function of B7-H3 and inhibit leukemia growth in vivo.

This data suggest that both T-1A5 and ChT-1A5 bind to peptide 5 and peptide 9 of the B7-H3 protein, which cover most of the FG loop and IgC domain. These data provide the mechanism by which the T-1A5 antibody blocks immunomodulatory functions of B7-H3. Considering that peptide 5 and peptide 9 are well separated and do not have any overlapping sequences, it is possible that the antibodies' epitope is a conformational epitope rather than a sequential epitope. These unique binding characteristics make this antibody a novel immune checkpoint inhibitor for cancer therapy. However, identification of the B7-H3 receptor might provide more clues on the effects of this antibody on immune cell activation, which is a subject of further investigation.

ADCC is a mechanism of cell-mediated immune defense through the antibody Fc region (55). NK cells are key components in the ADCC function of the antibody Fc (56). Several investigators have tested anti-B7-H3 antibodies in combination with NK cells and found that this combination reduced tumor progression through ADCC in solid tumors (10, 54). These results revealed for the first time that treatment with ChT-1A5 combined with NK cells substantially delays progression and apoptosis of leukemic cells by enhancing ADCC in AML cell lines and primary cells. Moreover, treatment of AML PDXs with ChT-1A5 in combination with activated NK cells synergistically decreased leukemia growth and increased mouse survival, in accordance with reports of the effects of anti-B7-H3 treatment in solid tumors in vivo. Since the antibody has blocking activity, the chimeric antibody could induce cell death by activation of NK and T cell-mediated cellular cytotoxicity as well as induce ADCC through Fc binding to the Fc receptor on the NK cells. Its effect on inducing macrophage-mediated antibody-dependent cell phagocytosis (ADCP) needs further investigation.

In conclusion, this study provides a proof-of-concept for the efficacy of targeting B7-H3 in AML. The inventors demonstrated that blocking of B7-H3 resulted in alteration of B7-H3 immunomodulatory functions and induces NK cell-mediated apoptosis. Also, targeting B7-H3 with ChT-1A5 antibody induced ADCC in leukemia cells. Therefore, the present study suggests that NK cells have potential to improve anti-17-B3 mAb therapeutic efficacy through ADCC. The xenograft and PDX model studies also suggested that B7-H3 is a novel immune checkpoint molecule in AML; therefore, the combinationofChT-A antibody with activatedNK cells couldpotentially benefit AML patients, especially high-risk patients.

### E. Tables

Supplementary Table 1. Association of B7-H3 expression with baseline parameters.

| | | | B7-H3 (MFI) | |
|---|---|---|---|---|
| | | | Mean ± Standard | |
| Parameters | | N | Deviation | P value |
| Gender | Female | 25 | 4457 ± 2676 | 0.203 |
| | Male | 34 | 3714 ± 1755 | |
| Subtype | M0 | 6 | 4729 ± 4300 | 0.48 |
| | M1 | 28 | 4010 ± 1606 | |
| | M2 | 14 | 3689 ± 1917 | |
| | Others | 11 | 3547 ± 2074 | |
| Treatment | No | 17 | 3908 ± 1828 | 0.79 |
| | Yes | 42 | 4078 ± 2357 | |
| ELN score | 1. Good | 12 | 2994 ± 1024 | 0.05 |
| | 2. Intermediate | 12 | 3532 ± 1865 | |
| | 3. Adverse | 35 | 4554 ± 2463 | |
| Death | No | 24 | 3351 ± 1640 | 0.04 |
| | Yes | 35 | 4494 ± 2432 | |
| Relapse | No | 43 | 4087 ± 2323 | 0.887 |
| | Yes | 16 | 3873 ± 1903 | |

Supplementary Table 2. Clinical parameters of AML PDX models.

| S. No. | PID | Age | Sex | Subtype | Cytogenetics/Mutations |
|---|---|---|---|---|---|
| 1 | 194-A | 56 | M | M5a | 46XY, inversion 7, FLT3-ITD |
| 2 | 452-A | 42 | F | M0 | t (6:11)(q27; q23), MLL |
| | | | | | rearrangement |
| 3 | 882-A | 68 | M | M5a | Diploid, FLT3-ITD |
| 4 | 778-A | 72 | M | M1 | Diploid, MLL, |
| | | | | | NPM1, DNMT3, IDH1, |
| | | | | | FLT3-ITD, NRAS |

### Example 2: B7-H3 Antibody (T-1A5) Induces NK- and T Cell-Mediated Cytotoxicity in Breast Cancer Cells

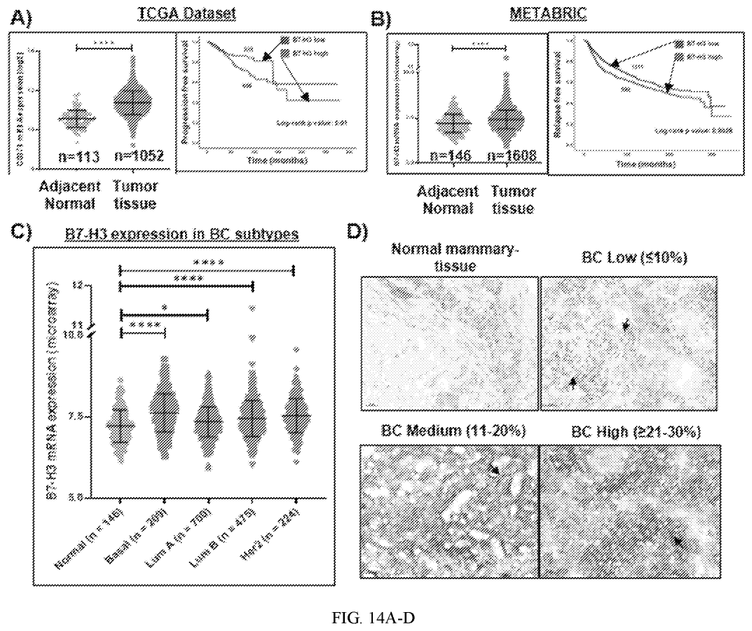

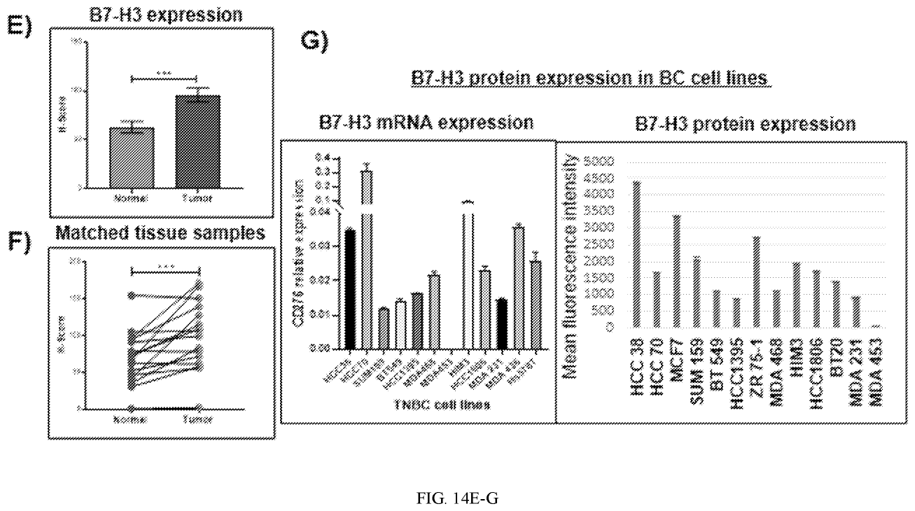

B7-H3 is upregulated in primary breast tumors and BC cell lines. To investigate the expression of B7-H3 in primary breast tumors, the inventors evaluated RNA-seq data from over 1000 primary and metastatic breast tumors and adjacent normal tissues from the TCGA and metabric databases. The inventors found that B7-H3 was significantly overexpressed (P<0.0001) in these tumors (FIG. 14A, B). A survival analysis by the log-rank test indicated that patients with B7-H3^{high} tumors had significantly lower rates of progression-free (P=0.01; FIG. 14A) and relapse-free (P=0.0026; FIG. 14B) survival than patients with B7-H3^{low} tumors. Moreover, B7-H3 is significantly upregulated in all of the BC subtypes including basal, luminal A, luminal B and Her2-enriched with highest expression in basal type BC (FIG. 14C). Next, to assess B7-H3 protein expression in BC, the inventors performed immunohistochemistry on frozen primary tumor tissues (n=50) and adjacent normal tissue (n=23) from TNBC patients. In line with the findings from publicly available datasets, the inventors observed variable B7-H3 expression in TNBC patient samples. Out of 50 samples, ˜45 cases (90%) had elevated levels of B7-H3 staining, with variable staining intensity (P<0.001; FIG. 14D). In contrast, mammary ducts in the adjacent normal tissue had low or no staining for B7-H3 (FIG. 14D, E). Compared to the matched normal tissue, B7-H3 expression was substantially higher in tumor tissue, suggesting that B7-H3 is a tumor-specific marker in TNBC patients (N=16, P<0.001; FIG. 14F). Moreover, the inventors assessed B7-H3 expression in over 13 BC cell lines, including TNBC and ER⁺, PR⁺, and HER2⁺ cell lines, as well as TNBC PDX-derived cells. Relative mRNA quantification and flow cytometry analysis demonstrated variable levels of B7-H3 expression in BC cell lines (FIG. 14G). Interestingly, both mRNA and protein expression of B7-H3 were found to be absent in MDA-MB-453 TNBC cells (FIG. 14G). These results suggest that B7-H3 is upregulated in several BC subtypes and is associated with poor overall survival.

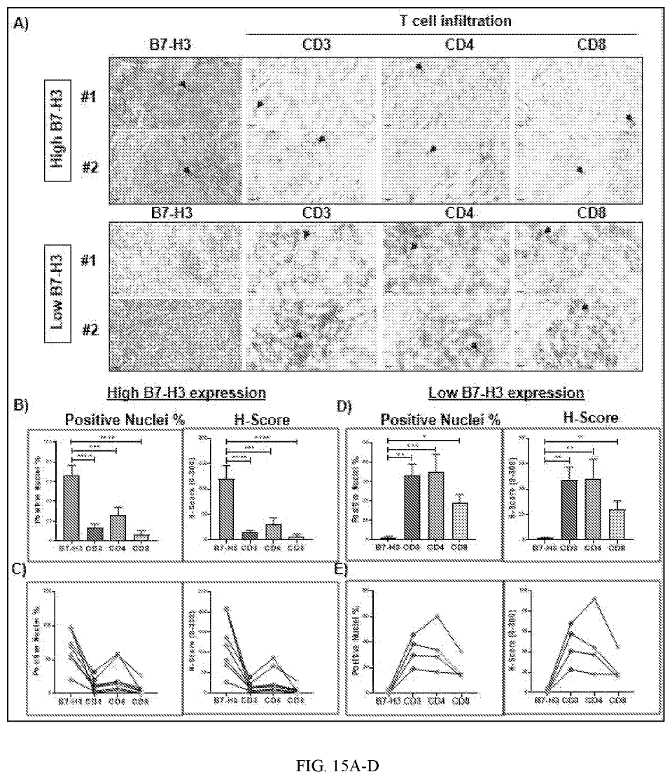

B7-H3 inhibits T-cell infiltration into primary breast tumors. To investigate the immunomodulatory effect of B7-H3 in BC, the inventors performed immunohistochemistry for T-cell markers in subsets of frozen patient tissues with high and low levels of B7-H3 expression. Expression of T-cell markers such as CD3, CD4, and CD8 was assessed in these samples. Interestingly, the inventors found an inverse correlation between B7-H3 expression and expression levels of CD3⁺, CD4⁺, and CD8⁺ T cells. Patients with high B7-H3 expression had significantly lower numbers of CD3⁺, CD4⁺, and CD8⁺ T-cell, whereas patients with low B7-H3 expression had significantly higher numbers (P<0.001; FIG. 15A, B, D). Quantitative analysis demonstrated that in comparison to other T-cell markers, B7-H3 expression level had the most significant impact on CD3⁺ T cells in these patients (P<0.0001; FIG. 15B-E). The inventors also discovered that B7-H3 directly modulated T-cell infiltration in primary TNBC tissues by analyzing matched expression of B7-H3 with T-cell markers in the same tissue samples. (FIG. 15C, E). These findings indicate that B7-H3 plays an immunomodulatory role by suppressing immune activation and infiltration of T cells into the TME.

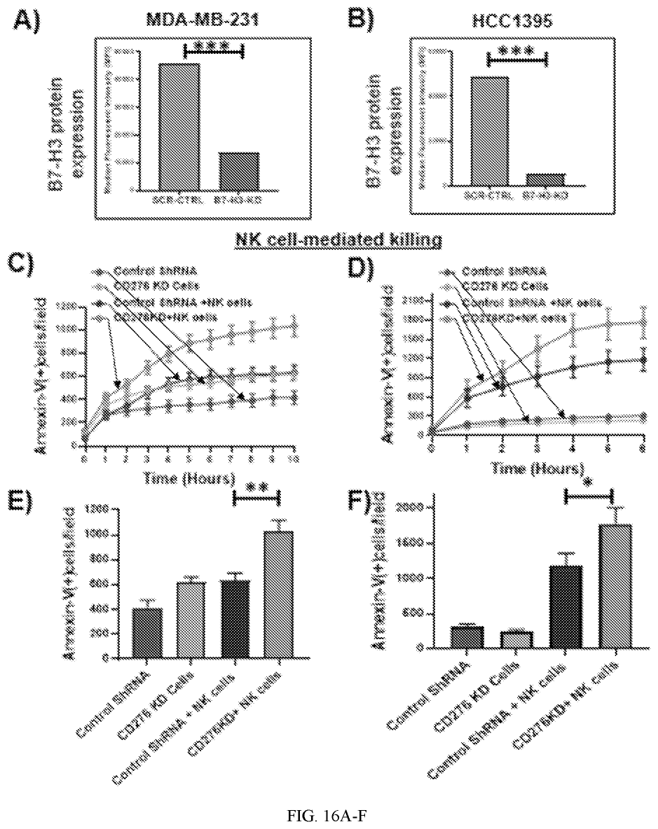

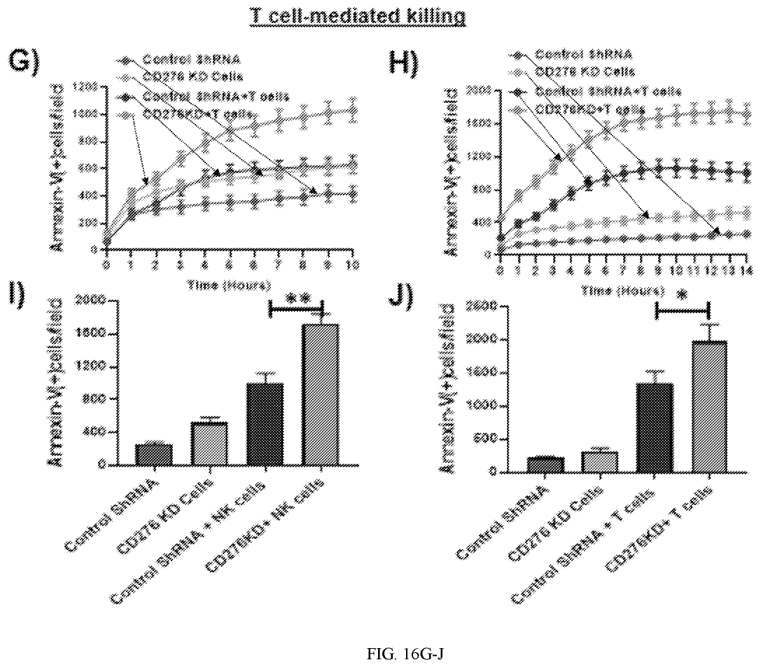

Inhibition of B7-H3 expression enhances NK- and T cell-mediated killing of BC cells. To investigate the role of B7-H3 in immunomodulation in BC cells, the inventors performed stable lentiviral knockdown (KD) of B7-H3 in MDA-MB-231 and HCC1395 BC cell lines. B7-H3 expression analysis by flow cytometry revealed that there was 70% to 80% knockdown in both cell lines (FIG. 16A, B). Next, to determine the effect of B7-H3 knockdown on NK- and T-cell activity, the inventors co-cultured control and B7-H3-KD BC cells in the presence and absence of NK and T cells and measured the induction of apoptosis in BC cells through annexin V binding using an IncuCyte live-cell imaging system. The inventors found a time-dependent increase in NK cell-mediated apoptosis in both MDA-MB-231 and HCC1395 cells. Interestingly, B7-H3 knockdown significantly enhanced NK cell-induced apoptosis compared to control shRNA-treated cells in both cell lines (FIG. 16C, D). Quantitative analysis revealed a 40% to 60% increase in NK cell-mediated killing in MDA-MB-231 and HCC1395 B7-H3-knockdown cell lines (P<0.001 and P<0.01) (FIG. 16E, F). Moreover, the inventors found significantly higher T cell-mediated killing in B7-H3-knockdown MDA-MB 231 and HCC1395 BC cells than in control shRNA-treated cells in a time-dependent manner (FIG. 16G-J). Overall, these findings suggest that the knockdown of B7-H3 stimulates immune-cell function in BC.

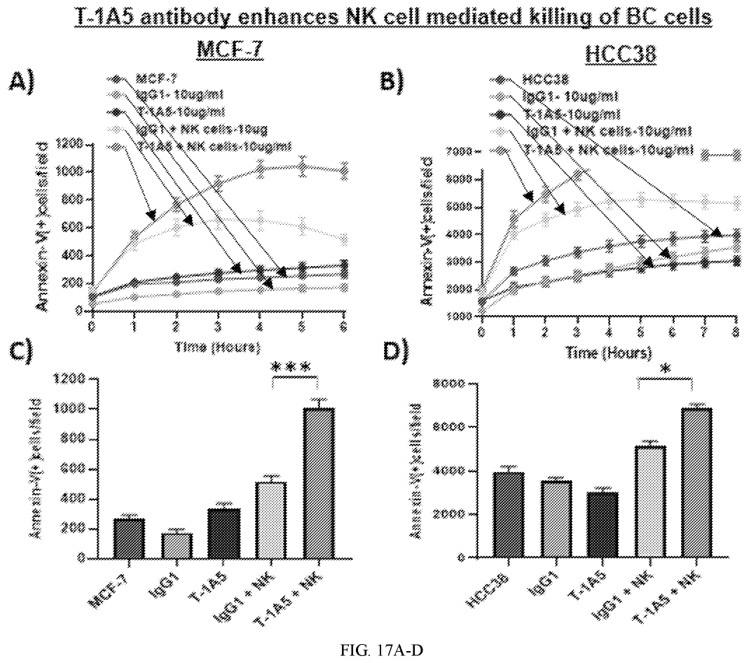

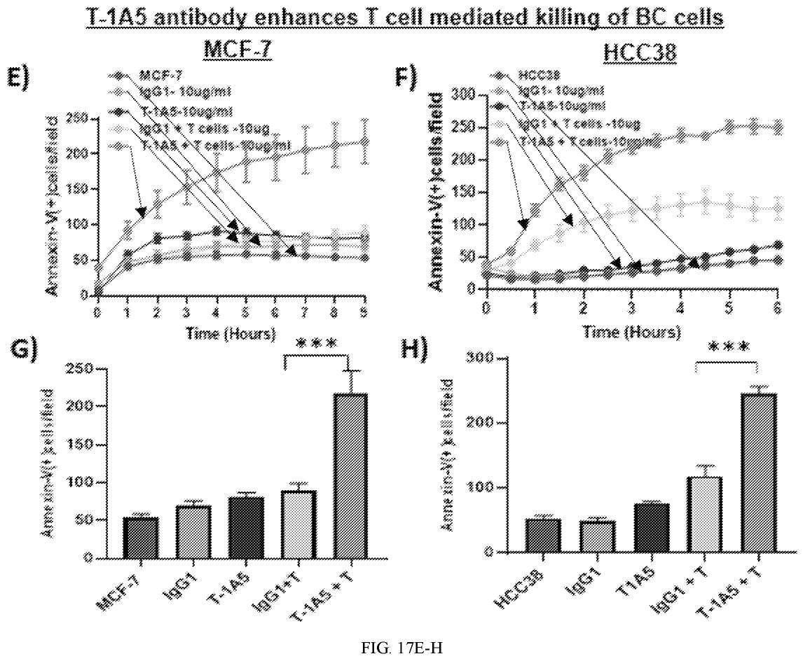

Anti-B7-H3 blocking antibody enhances immune cell-mediated apoptosis in BC cell lines. To block the immunomodulatory functions of B7-H3, the inventors generated a novel antibody (clone T-1A5, isotype IgG1). To determine the effect of this T-1A5 antibody on immune-cell function, the inventors co-cultured BC cells (MCF-7 and HCC38) with and without NK cells at a 15:1 NK-to-target cell ratio in the presence or absence of the T-1A5 antibody. NK cell-induced apoptosis of BC cells was measured by imaging annexin V binding on red fluorescent protein (RFP)-expressing BC cell lines using the IncuCyte live-cell imaging system. The inventors found that cells treated with T1-A5 in combination with activated NK cells showed a significant increase in annexin V binding within a few hours of co-culture, reaching a plateau in the next 10-12 hours. In contrast, the inventors did not observe significant apoptosis in IgG1-treated samples (isotype control) (FIG. 17A-D). Next, to determine the effect of B7-H3 blockade on T cell-induced cell death, the inventors co-cultured RFP-expressing HCC38 and MCF-7 cells with T cells at a 10:1 T cell-to-target cell ratio in the presence of the anti-B7-H3 antibody T-1A5 or the IgG1 control antibody. The inventors found significantly higher T cell-mediated apoptosis in HCC38 and MCF-7 cells treated with the T-1A5 antibody compared to the IgG1 control (FIG. 17E-H). Overall, these results indicate that blockade of the immunomodulatory functions of B7-H3 using the anti-B7-H3 antibody T-1A5 enhances immune cell-mediated apoptosis in BC cells.

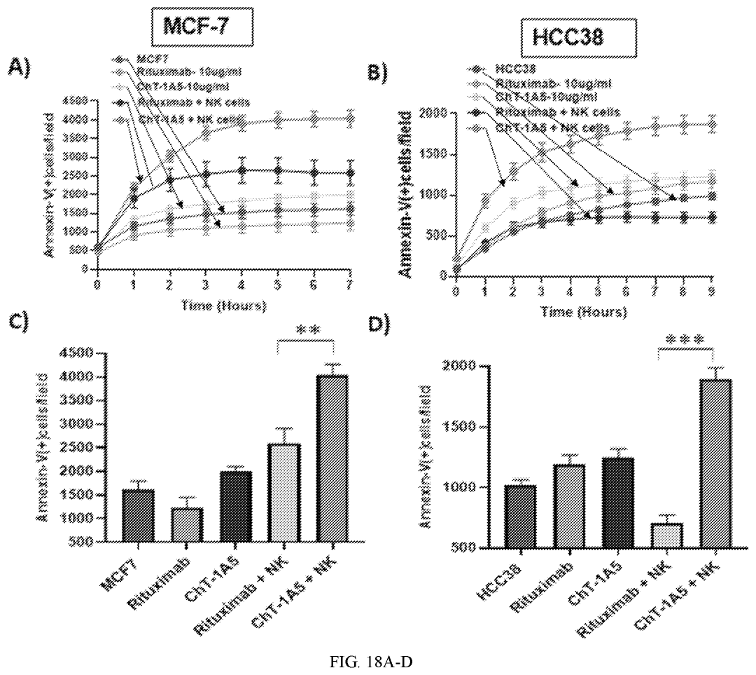

Chimeric T-1A5 antibody induced NK cell-mediated ADCC in BC cell lines. To develop the T-1A5 antibody as a therapeutic tool for B7-H3-positive BC, the inventors generated a human-mouse chimeric antibody (chT-1A5) and analyzed its effect on the induction of ADCC in B7-H3-positive BC. The inventors treated BC cell lines with high B7-H3 expression (MCF7 and HCC38) with chT-1A5 or rituximab (10 μg/mL) in the presence or absence of activated NK cells at a 15:1 NK-to-target cell ratio. Rituximab is a chimeric anti-CD20 antibody that was used as a nonspecific control antibody. The inventors found a significant increase in apoptosis in BC cells treated with the chT-1A5 antibody and NK cells, whereas treatment with rituximab and NK cells did not show significant outcomes (FIG. 18A, B). As shown in FIGS. 18C and 18D, after 10 hours, treatment with chT-1A5 or rituximab alone did not affect the killing of BC cells, whereas chT-1A5 with activated NK cells induced significant apoptosis in MCF-7 and HCC38 cells in a time-dependent manner. These findings suggest that the chT-1A5 antibody induces ADCC in BC cell lines expressing B7-H3.

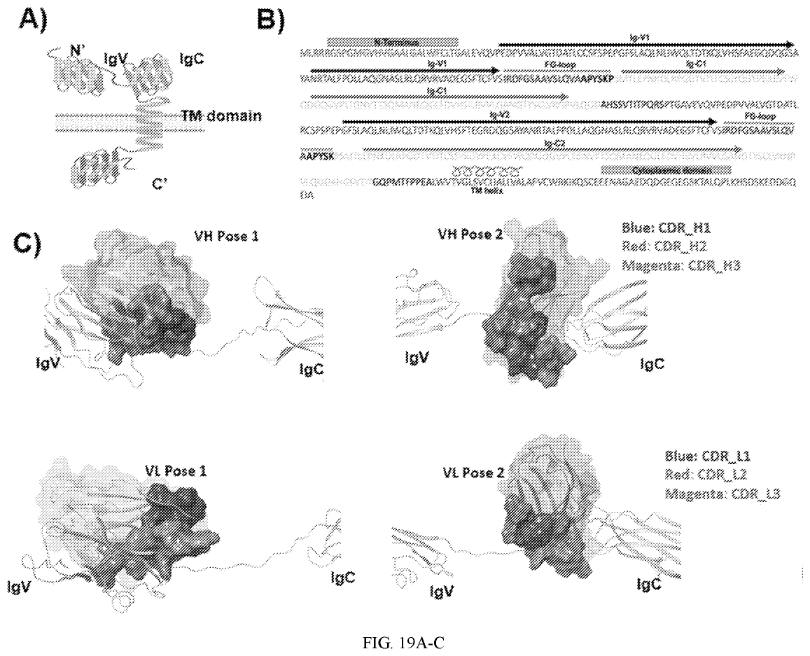

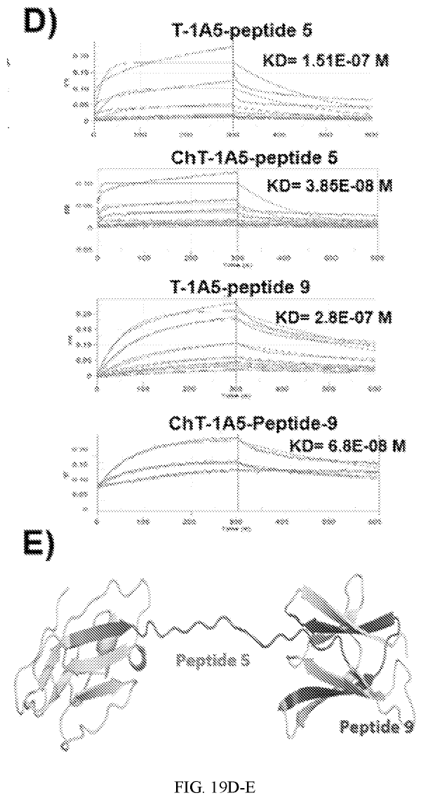

Monoclonal antibodies T-1A5 and chT-1A5 bind to the FG loop region of B7-H3. A recent study found that the FG loop region on the mouse B7-H3 protein is responsible for B7-H3's immunomodulatory function. To identify the antibody binding sites for the T-1A5 antibody on the B7-H3 protein, the inventors marked the already-reported structural elements of human B7-H3 (FIG. 19A, B). Next, the inventors generated a structural homology modeling of B7-H3 based on the crystal structure of murine B7-H3 as a template to predict how the variable heavy (VH) or variable light chain domains of T-1A5 antibody were docked on the B7-H3 protein (FIG. 19C). The 2 best poses of the VH and VL domains that were docked on either the IgV or IgC domains of B7-H3 were further analyzed. Interestingly, pose 1 of VH or VL was docked at the intersection of IgV and the FG loop, while pose 2 represented binding towards the FG loop intersection with IgC. These predicted interactions indicated binding of the T-1A5 antibody to specific domains of B7-H3. Further, the inventors performed epitope mapping to identify the antibody binding sites on B7-H3. The inventors generated 10 peptides from the extracellular domain of B7-H3 with a span of 25 amino acids and an overlap of 5 amino acids with each subsequent peptide. Interestingly, a biolayer interferometry assay using the Octet system showed that peptide 5 and peptide 9 had strongest binding affinity with both the mouse antibody (T-1A5) and the chimeric antibody (chT-1A5; FIG. 19D). Strikingly, further analysis of the B7-H3 model showed that peptide 5 (red) was present in the IgV domain spanning through the FG loop and that peptide 9 (blue) was present in the IgC domain (FIG. 19E). Taken together, these results suggest that the FG loop of B7-H3 is the binding site for both T-1A5 and chT-1A5 antibodies.

### Example 3: Blocking B7-H3 Using the T-1A5 Antibody Synergistically Enhances Venetocolax-Induced Apoptosis in AML Cells

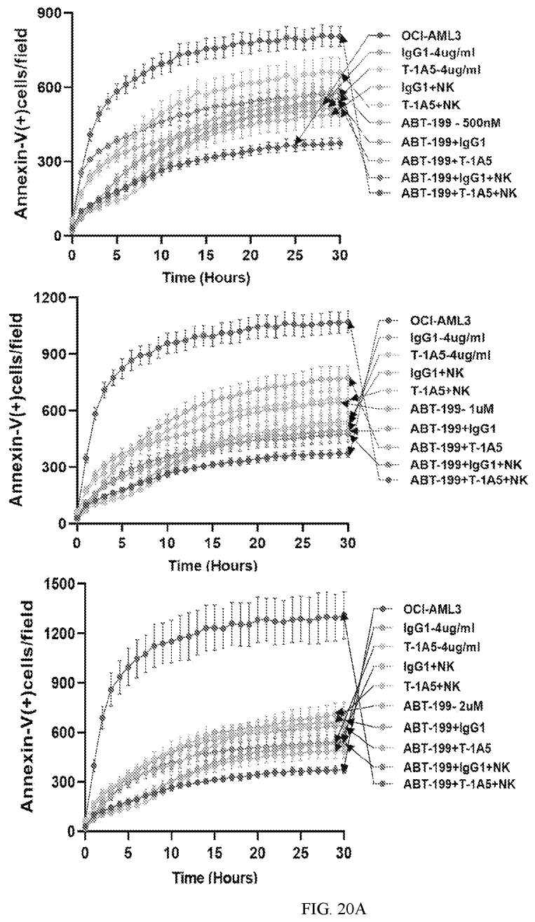

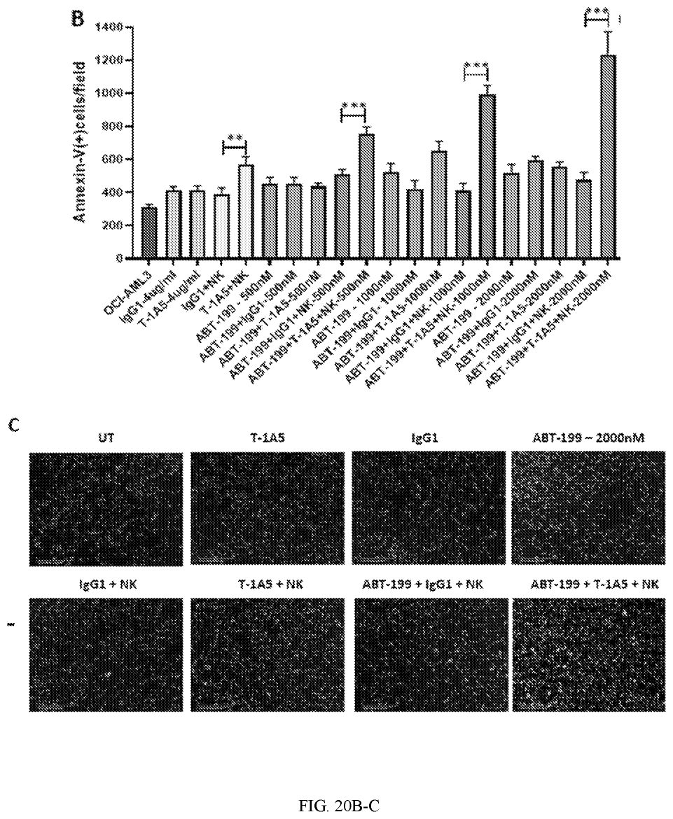

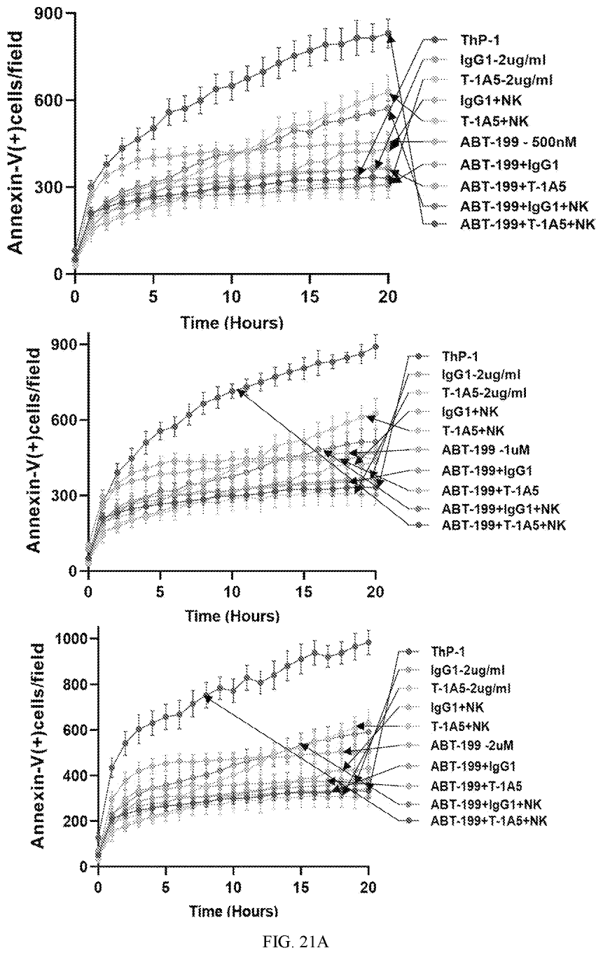

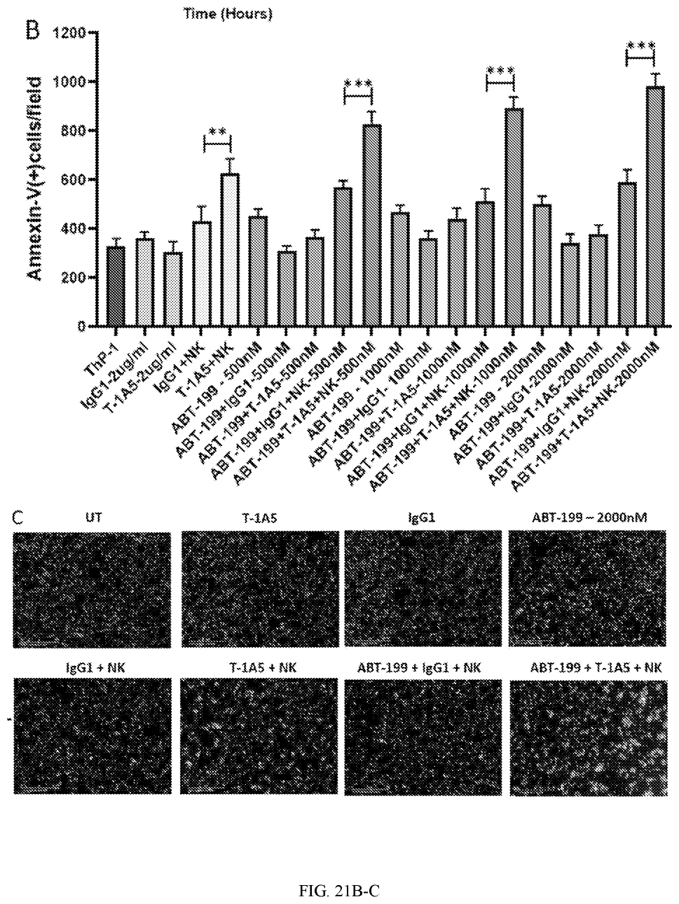

The BCL2 inhibitor, Venetoclax has recently been shown to enhance NK cell-mediated killing of cancer cells. Here we tested the combination of B7-H3 blocking antibody (T-1A5) with venetoclax to measure NK cell-mediated killing of AML cells. OCI-AML3 or THP1 cells labeled with the Cytolight-red reagent were treated with T-1A5 alone or in combination with ABT-199 (Venetoclax) at different concentrations in the presence or absence of NK cells at a 4:1 NK:target cell ratio. We observed a dose-dependent synergistic anti-leukemic effect with the combination of T-1A5 and Venetoclax against B7-H3+ AML cells, while cells treated with IgG1 and ABT-199 plus NK cells showed no significant increase in apoptosis (FIG. 20A). A bar graph representing annexin-v binding at the 8-hour time point for all treatment groups has been shown in FIG. 20B (P<0.001). Representative images of annexin-v binding on OCI-AML3 and THP-1 cells undergoing apoptosis (green staining) at 24-hour time points from different treatment conditions are shown in FIG. 20C. These data suggest that the combination of T-1A5 and venetoclax synergistically enhance NK cell-mediated killing of AML cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the references cited herein, to the extent that they provide exemplary procedural or other details supplementary to those set forth herein, are specifically incorporated herein by reference.
1. Jordan C T. Unique molecular and cellular features of acute myelogenous leukemia stem cells. Leukemia. 2002; 16(4):559-62.
2. Yamamoto J F, Goodman M T. Patterns of leukemia incidence in the United States by subtype and demographic characteristics, 1997-2002. Cancer Causes Control. 2008; 19(4):379-90.
3. Shallis R M, Wang R, Davidoff A, Ma X, Zeidan A M. Epidemiology of acute myeloid leukemia: Recent progress and enduring challenges. Blood Rev. 2019; 36:70-87.
4. Shah A, Andersson T M, Rachet B, Bjorkholm M, Lambert PC. Survival and cure of acute myeloid leukaemia in England, 1971-2006: a population-based study. Br J Haematol. 2013; 162(4):509-16.
5. Yang H, Rosove M H, Figlin R A. Tumor lysis syndrome occurring after the administration of rituximab in lymphoproliferative disorders: high-grade non-Hodgkin's lymphoma and chronic lymphocytic leukemia. Am J Hematol. 1999; 62(4):247-50.
6. Heinhuis K M, Ros W, Kok M, Steeghs N, Beijnen J H, Schellens J H M. Enhancing antitumor response by combining immune checkpoint inhibitors with chemotherapy in solid tumors. Ann Oncol. 2019; 30(2):219-35.
7. Sun J, Fu F, Gu W, Yan R, Zhang G, Shen Z, et al. Origination of new immunological functions in the costimulatory molecule B7-H3: the role of exon duplication in evolution of the immune system. PLoS One. 2011; 6(9):e24751.
8. Steinberger P, Majdic O, Derdak S V, Pfistershammer K, Kirchberger S, Klauser C, et al. Molecular characterization of human 41g-B7-H3, a member of the B7 family with four Ig-like domains. J Immunol. 2004; 172(4):2352-9.
9. Vigdorovich V, Ramagopal U A, Lázár-Molnár E, Sylvestre E, Lee J S, Hofmeyer K A, et al. Structure and T cell inhibition properties of B7 family member, B7-H3. Structure. 2013; 21(5):707-17.
10. Lee Y H, Martin-Orozco N, Zheng P, Li J, Zhang P, Tan H, et al. Inhibition of the B7-H3 immune checkpoint limits tumor growth by enhancing cytotoxic lymphocyte function. Cell Res. 2017; 27(8):1034-45.
11. Leitner J, Klauser C, Pickl W F, Stockl J, Majdic O, Bardet A F, et al. B7-H3 is a potent inhibitor of human T-cell activation: No evidence for B7-H3 and TREML2 interaction. Eur J Immunol. 2009; 39(7):1754-64.
12. Prasad D V, Nguyen T, Li Z, Yang Y, Duong J, Wang Y, et al. Murine B7-H3 is a negative regulator of T cells. J Immunol. 2004; 173(4):2500-6.
13. Suh W K, Gajewska B U, Okada H, Gronski M A, Bertram E M, Dawicki W, et al. The B7 family member B7-H3 preferentially down-regulates T helper type 1-mediated immune responses. Nat Immunol. 2003; 4(9):899-906.
14. Ueno T, Yeung M Y, McGrath M, Yang S, Zaman N, Snawder B, et al. Intact B7-H3 signaling promotes allograft prolongation through preferential suppression of Th1 effector responses. Eur J Immunol. 2012; 42(9):2343-53.
15. Chapoval A I, Ni J, Lau J S, Wilcox R A, Flies D B, Liu D, et al. B7-H3: a costimulatory molecule for T cell activation and IFN-gamma production. Nat Immunol. 2001; 2(3):269-74.
16. Jiang J, Jiang J, Liu C, Zhang G, Gao L, Chen Y, et al. Enhancement of membrane B7-H3 costimulatory molecule but reduction of its soluble form in multiple sclerosis. J Clin Immunol. 2013; 33(1):118-26.
17. Inamura K, Yokouchi Y, Kobayashi M, Sakakibara R, Ninomiya H, Subat S, et al. Tumor B7-H3 (CD276) expression and smoking history in relation to lung adenocarcinoma prognosis. Lung Cancer. 2017; 103:44-51.
18. Loos M, Hedderich D M, Friess H, Kleeff J. B7-h3 and its role in antitumor immunity. Clin Dev Immunol. 2010; 2010:683875.
19. Picarda E, Ohaegbulam K C, Zang X. Molecular Pathways: Targeting B7-H3 (CD276) for Human Cancer Immunotherapy. Clin Cancer Res. 2016; 22(14):3425-31.
20. Seaman S, Zhu Z, Saha S, Zhang X M, Yang M Y, Hilton M B, et al. Eradication of Tumors through Simultaneous Ablation of CD276/B7-H3-Positive Tumor Cells and Tumor Vasculature. Cancer Cell. 2017; 31(4):501-15 e8.
21. Bin Z, Guangbo Z, Yan G, Huan Z, Desheng L, Xueguang Z. Overexpression of B7-H3 in CD133+ colorectal cancer cells is associated with cancer progression and survival in human patients. J Surg Res. 2014; 188(2):396-403.
22. Hofmeyer K A, Ray A, Zang X. The contrasting role of B7-H3. Proc Natl Acad Sci USA. 2008; 105(30):10277-8.
23. Benzon B, Zhao S G, Haffner M C, Takhar M, Erho N, Yousefi K, et al. Correlation of B7-H3 with androgen receptor, immune pathways and poor outcome in prostate cancer: an expression-based analysis. Prostate Cancer Prostatic Dis. 2017; 20(1):28-35.
24. Qin X, Zhang H, Ye D, Dai B, Zhu Y, Shi G. B7-H3 is a new cancer-specific endothelial marker in clear cell renal cell carcinoma. Onco Targets Ther. 2013; 6:1667-73.
25. Tang X, Zhao S, Zhang Y, Wang Y, Zhang Z, Yang M, et al. B7-H3 as a Novel CAR-T Therapeutic Target for Glioblastoma. Mol Ther Oncolytics. 2019; 14:279-87.
26. Nehama D, Di Ianni N, Musio S, Du H, Patane M, Pollo B, et al. B7-H3-redirected chimeric antigen receptor T cells target glioblastoma and neurospheres. EBioMedicine. 2019; 47:33-43.
27. Lu H, Shi T, Wang M, Li X, Gu Y, Zhang X, et al. B7-H3 inhibits the IFN-gamma-dependent cytotoxicity of Vgamma9Vdelta2 T cells against colon cancer cells. Oncoimmunology. 2020; 9(1):1748991.
28. Loo D, Alderson R F, Chen F Z, Huang L, Zhang W, Gorlatov S, et al. Development of an Fc-enhanced anti-B7-H3 monoclonal antibody with potent antitumor activity. Clin Cancer Res. 2012; 18(14):3834-45.
29. Fauci J M, Sabbatino F, Wang Y, Londono-Joshi A I, Straughn J M, Jr., Landen C N, et al. Monoclonal antibody-based immunotherapy of ovarian cancer: targeting ovarian cancer cells with the B7-H3-specific mAb 376.96. Gynecol Oncol. 2014; 132(1):203-10.
30. Kasten B B, Arend R C, Katre A A, Kim H, Fan J, Ferrone S, et al. B7-H3-targeted (212)Pb radioimmunotherapy of ovarian cancer in preclinical models. Nucl Med Biol. 2017; 47:23-30.
31. Yuan B, El Dana F, Ly S, Yan Y, Ruvolo V, Shpall E J, et al. Bone marrow stromal cells induce an ALDH+ stem cell-like phenotype and enhance therapy resistance in AML through a TGF-β-p38-ALDH2 pathway. PLoS One. 2020; 15(11):e0242809.
32. Battula V L, Treml S, Bareiss P M, Gieseke F, Roelofs H, de Zwart P, et al. Isolation of functionally distinct mesenchymal stem cell subsets using antibodies against CD56, CD271, and mesenchymal stem cell antigen-1. Haematologica. 2009; 94(2):173-84.
33. Battula V L, Le P M, Sun J C, Nguyen K, Yuan B, Zhou X, et al. AML-induced osteogenic differentiation in mesenchymal stromal cells supports leukemia growth. JCI Insight. 2017; 2(13).
34. Hu J, Vien L T, Xia X, Bover L, Li S. Generation of a monoclonal antibody against the glycosylphosphatidylinositol-linked protein Rae-1 using genetically engineered tumor cells. Biol Proced Online. 2014; 16(1):3.
35. Qin H, Wei G, Sakamaki I, Dong Z, Cheng W A, Smith D L, et al. Novel BAFF-Receptor Antibody to Natively Folded Recombinant Protein Eliminates Drug-Resistant Human B-cell Malignancies In Vivo. Clin Cancer Res. 2018; 24(5):1114-23.
36. Voo K S, Bover L, Harline M L, Vien L T, Facchinetti V, Arima K, et al. Antibodies targeting human OX40 expand effector T cells and block inducible and natural regulatory T cell function. J Immunol. 2013; 191(7):3641-50.
37. Uhlen M, Bandrowski A, Carr S, Edwards A, Ellenberg J, Lundberg E, et al. A proposal for validation of antibodies. Nat Methods. 2016; 13(10):823-7.
38. Yeung T L, Leung C S, Yip K P, Sheng J, Vien L, Bover L C, et al. Anticancer Immunotherapy by MFAP5 Blockade Inhibits Fibrosis and Enhances Chemosensitivity in Ovarian and Pancreatic Cancer. Clin Cancer Res. 2019; 25(21):6417-28.
39. Vera J, Savoldo B, Vigouroux S, Biagi E, Pule M, Rossig C, et al. T lymphocytes redirected against the kappa light chain of human immunoglobulin efficiently kill mature B lymphocyte-derived malignant cells. Blood. 2006; 108(12):3890-7.
40. Pule M A, Straathof K C, Dotti G, Heslop H E, Rooney C M, Brenner M K. A chimeric T cell antigen receptor that augments cytokine release and supports clonal expansion of primary human T cells. Mol Ther. 2005; 12(5):933-41.
41. Dohner H, Estey E, Grimwade D, Amadori S, Appelbaum F R, Buchner T, et al. Diagnosis and management of AML in adults: 2017 ELN recommendations from an international expert panel. Blood. 2017; 129(4):424-47.
42. Tyner J W, Tognon C E, Bottomly D, Wilmot B, Kurtz S E, Savage S L, et al. Functional genomic landscape of acute myeloid leukaemia. Nature. 2018; 562(7728):526-31.
43. Ly S, Yuan B, Grimm S, Andreeff M, Buhring H J, Battula V L, B7—H3,an immune checkpoint proteinis overexpressed in AML and the blocking monoclonal antibodies enhanceNKcell-mediated apoptosis in AMLcells, in: clinical Research (excludingClinicalTrials). American Association for Cancer Research AM2019-3248:3248-3248
44. Curiel T J, Wei S, Dong H, Alvarez X, Cheng P, Mottram P, et al. Blockade of B7-H1 improves myeloid dendritic cell-mediated antitumor immunity. Nat Med. 2003; 9(5):562-7.
45. McDermott D F, Atkins M B. PD-1 as a potential target in cancer therapy. Cancer Med. 2013; 2(5):662-73.
46. Guery T, Roumier C, Berthon C, Renneville A, Preudhomme C, Quesnel B. B7-H3 protein expression in acute myeloid leukemia. Cancer Med. 2015; 4(12):1879-83.
47. Toffalori C, Zito L, Gambacorta V, Riba M, Oliveira G, Bucci G, et al. Immune signature drives leukemia escape and relapse after hematopoietic cell transplantation. Nat Med. 2019; 25(4):603-11.
48. Leitner J, Klauser C, Pickl W F, Stöckl J, Majdic O, Bardet A F, et al. B7-H3 is a potent inhibitor of human T-cell activation: No evidence for B7-H3 and TREML2 interaction. Eur J Immunol. 2009; 39(7):1754-64.
49. Zhang J, Liu L, Han S, Li Y, Qian Q, Zhang Q, et al. B7-H3 is related to tumor progression in ovarian cancer. Oncol Rep. 2017; 38(4):2426-34.
50. Chen L, Chen J, Xu B, Wang Q, Zhou W, Zhang G, et al. B7-H3 expression associates with tumor invasion and patient's poor survival in human esophageal cancer. Am J Transl Res. 2015; 7(12):2646-60.
51. Brustmann H, Igaz M, Eder C, Brunner A. Epithelial and tumor-associated endothelial expression of B7-H3 in cervical carcinoma: relation with CD8+ intraepithelial lymphocytes, FIGO stage, and phosphohistone H3 (PHH3) reactivity. Int J Gynecol Pathol. 2015; 34(2):187-95.
52. Lu H, Shi T, Wang M, Li X, Gu Y, Zhang X, et al. B7-H3 inhibits the IFN-γ-dependent cytotoxicity of Vγ9Vδ2 T cells against colon cancer cells. Oncoimmunology. 2020; 9(1):1748991.
53. Cai D, Li J, Liu D, Hong S, Qiao Q, Sun Q, et al. Tumor-expressed B7-H3 mediates the inhibition of antitumor T-cell functions in ovarian cancer insensitive to PD-1 blockade therapy. Cell Mol Immunol. 2020; 17(3):227-36.
54. Ahmed M, Cheng M, Zhao Q, Goldgur Y, Cheal S M, Guo H F, et al. Humanized Affinity-matured Monoclonal Antibody 8H9 Has Potent Antitumor Activity and Binds to F G Loop of Tumor Antigen B7-H3. J Biol Chem. 2015; 290(50):30018-29.
55. Ochoa M C, Minute L, Rodriguez I, Garasa S, Perez-Ruiz E, Inogés S, et al. Antibody-dependent cell cytotoxicity: immunotherapy strategies enhancing effector N K cells. Immunol Cell Biol. 2017; 95(4):347-55.
56. Seidel U J, Schlegel P, Lang P. Natural killer cell mediated antibody-dependent cellular cytotoxicity in tumor immunotherapy with therapeutic antibodies. Front Immunol. 2013; 4:76.

What is claimed is:

1. An antibody or antigen binding fragment comprising a heavy chain variable region (VH) and a light chain variable region (VL), wherein the VH comprises a HCDR1, HCDR2, and HCDR3 from the VH of SEQ ID NO:3 and wherein the VL comprises a LCDR1, LCDR2, and LCDR3 from the VL of SEQ ID NO:13.

2. An antibody or antigen binding fragment comprising a VH and a VL, wherein the VH comprises a HCDR1, HCDR2, and HCDR3 having at least 80% sequence identity with a HCDR1, HCDR2, and HCDR3 from the VH of SEQ ID NO:3 and wherein the VL comprises a LCDR1, LCDR2, and LCDR3 having at least 80% sequence identity with a LCDR1, LCDR2, and LCDR3 from the VL of SEQ ID NO:13.

3. An antibody or antigen binding fragment comprising a VH and a VL, wherein the VH comprises a HCDR1, HCDR2, and HCDR3 comprising the amino acid sequence of SEQ ID NOS:4-6, respectively, and wherein the VL comprises a LCDR1, LCDR2, and LCDR3 comprising the amino acid sequence of SEQ ID NOS:14-16, respectively.

4. An antibody or antigen binding fragment comprising a VH and a VL, wherein the VH comprises a HCDR1, HCDR2, and HCDR3 having at least 80% sequence identity to SEQ ID NOS:4-6, respectively, and wherein the VL comprises a LCDR1, LCDR2, and LCDR3 having at least 80% sequence identity to SEQ ID NOS:14-16, respectively.

5. The antibody or antigen binding fragment of any one of claims 1-4, wherein the VH comprises an amino acid sequence with at least 80% sequence identity to SEQ ID NO:3 and/or the VL comprises an amino acid sequence with at least 80% sequence identity to SEQ ID NO:13.

6. The antibody or antigen binding fragment of any one of claims 1-5, wherein the antibody or antigen binding fragment comprises a heavy chain framework region 1-4 and light chain framework region 1-4 and wherein the heavy chain framework region 1-4 comprises an amino acid sequence with at least 80% sequence identity to SEQ ID NOS:7-10, respectively, and the light chain framework region 1-4 comprises an amino acid sequence with at least 80% sequence identity to SEQ ID NOS:17-20, respectively.

7. The antibody or antigen binding fragment of claim 6, wherein the heavy chain framework region 1-4 comprises the amino acid sequence of SEQ ID NOS:7-10, respectively, and the light chain framework region 1-4 comprises the amino acid sequence of SEQ ID NOS:17-20, respectively.

8. The antibody or antigen binding fragment of any one of claims 1-7, wherein the antibody or antigen binding fragment comprises an amino acid sequence with at least 70% sequence identity to one of SEQ ID NOS:1-20 and/or an amino acid sequence with 1 substitution relative to SEQ ID NOS:1-20.

9. The antibody of any one of claims 1-8, wherein the antibody comprises a heavy chain and a light chain and wherein the heavy chain comprises an amino acid sequence with at least 70% sequence identity to SEQ ID NO:1 and the light chain comprises an amino acid sequence with at least 70% sequence identity to SEQ ID NO:11.

10. The antibody or antigen binding fragment of any one of claims 1-9, wherein the antibody or antigen binding fragment comprises a signal peptide.

11. The antibody or antigen binding fragment of claim 10, wherein the signal peptide comprises SEQ ID NO:2 or 12 or an amino acid sequence having at least 80% sequence identity to SEQ ID NO:2 or 12.

12. The antibody of any one of claims 1-11, wherein the antibody is human, chimeric, or humanized.

13. The antibody or antigen-binding fragment of any one of claims 1-12, wherein the antibody, or antigen binding fragment binds B7H3 with a K_{D} of about 10⁻⁶ nM to about 10⁻¹² pM.

14. The antibody or antigen binding fragment of claim 13, wherein the antibody is a blocking antibody.

15. The antibody or antigen binding fragment of any one of claims 1-14, wherein the antibody or antigen binding fragment is a neutralizing antibody.

16. The antibody or antigen binding fragment of any one of claims 1-15, wherein the antibody is a human antibody, humanized antibody, recombinant antibody, chimeric antibody, an antibody derivative, a veneered antibody, a diabody, a monoclonal antibody, a single domain antibody, or a single chain antibody.

17. The antibody of claim 16, wherein the antibody is a chimeric antibody.

18. The antibody of claim 17, wherein the antibody comprises a heavy chain comprising the amino acid sequence of SEQ ID NO:113 or an amino acid sequence with at least 70% sequence identity to SEQ ID NO:113 and a light chain comprising the amino acid sequence of SEQ ID NO:114 or an amino acid sequence with at least 70% sequence identity to SEQ ID NO:114

19. The antigen binding fragment of any one of claims 1-15, wherein the antigen binding fragment is a single chain variable fragment (scFv), F(ab′)₂, Fab′, Fab, Fv, or rIgG.

20. A polypeptide comprising the antigen binding fragment of any one of claims 1-19.

21. A polypeptide comprising a single chain variable fragment (scFv) comprising a heavy chain variable region (VH) and a light chain variable region (VL) wherein the VH comprises a HCDR1, HCDR2, and HCDR3 from a heavy chain variable region of SEQ ID NO:3 and wherein the VL comprises a LCDR1, LCDR2, and LCDR3 from a VL of SEQ ID NO:13.

22. A polypeptide comprising a single chain variable fragment (scFv) comprising a VH and a VL wherein the VH comprises a HCDR1, HCDR2, and HCDR3 having at least 80% sequence identity with a HCDR1, HCDR2, and HCDR3 from a VH of SEQ ID NO:3 and wherein the VL comprises a LCDR1, LCDR2, and LCDR3 having at least 80% sequence identity with a LCDR1, LCDR2, and LCDR3 from a VL of SEQ ID NO:13.

23. A polypeptide comprising a single chain variable fragment (scFv) comprising a VH and a VL wherein the VH comprises a HCDR1, HCDR2, and HCDR3 comprising the amino acid sequence of SEQ ID NOS:4-6, respectively, and wherein the VL comprises a LCDR1, LCDR2, and LCDR3 comprising the amino acid sequence of SEQ ID NOS:14-16, respectively.

24. A polypeptide comprising a single chain variable fragment (scFv) comprising a VH and a VL wherein the VH comprises a HCDR1, HCDR2, and HCDR3 having at least 80% sequence identity to SEQ ID NOS:4-6, respectively, and wherein the VL comprises a LCDR1, LCDR2, and LCDR3 having at least 80% sequence identity to SEQ ID NOS:14-16, respectively.

25. The polypeptide of any one of claims 21-24, wherein the VH comprises an amino acid sequence with at least 80% sequence identity to SEQ ID NO:3 and/or the VL comprises an amino acid sequence with at least 80% sequence identity to SEQ ID NO:13

26. The polypeptide of any one of claims 21-25, wherein the scFv comprises a heavy chain framework region 1-4 and light chain framework region 1-4 and wherein the heavy chain framework region 1-4 comprises an amino acid sequence with at least 80% sequence identity to SEQ ID NOS:7-10, respectively, and the light chain framework region 1-4 comprises an amino acid sequence with at least 80% sequence identity to SEQ ID NOS:17-20, respectively.

27. The polypeptide of claim 26, wherein the heavy chain framework region 1-4 comprises the amino acid sequence of SEQ ID NOS:7-10, respectively, and the light chain framework region 1-4 comprises the amino acid sequence of SEQ ID NOS:17-20, respectively.

28. The polypeptide of any one of claims 21-27, wherein the VH is amino proximal to the VL.

29. The polypeptide of any one of claims 21-27, wherein the VL is amino proximal to the VH.

30. The polypeptide of any one of claims 21-29, wherein VH region and VL region are separated by a peptide linker.

31. The polypeptide of claim 31, wherein the peptide linker is a glycine-serine linker.

32. The polypeptide of claim 30 or 31, wherein the peptide linker is at least 4 amino acids.

33. The polypeptide of any one of claims 21-32, wherein the polypeptide further comprises a second scFv.

34. The polypeptide of claim 33, wherein the second scFv specifically binds to CD3.

35. The polypeptide of any one of claims 21-33, wherein the polypeptide further comprises a CD3 binding region.

36. The polypeptide of claim 35, wherein the CD3 binding region comprises a scFv that specifically binds to CD3.

37. A chimeric antigen receptor (CAR) comprising:
a) an extracellular binding domain comprising the polypeptide of any one of claims 21-36;
b) a transmembrane domain; and,
c) a cytoplasmic region comprising a costimulatory domain and a primary intracellular signaling domain.

38. The CAR of claim 37, wherein the cytoplasmic region further comprises a co-stimulatory region between the transmembrane domain and the cytoplasmic region.

39. The CAR of claim 37 or 32, wherein the transmembrane domain comprises a transmembrane domain of CD28.

40. The CAR of any one of claims 37-39, wherein the primary intracellular signaling domain comprises a CD28 or CD3 zeta signaling domain.

41. The CAR of any one of claims 37-40, wherein the CAR comprises a peptide spacer between the extracellular binding domain and the transmembrane domain.

42. The CAR of claim 41, wherein the peptide spacer comprises less than 50 amino acids.

43. The CAR of claim 41 or 42, wherein the peptide spacer comprises more than 50 amino acids.

44. The CAR of any one of claims 41-43, wherein the peptide spacer comprises the hinge region of an IgG molecule.

45. The CAR of any one of claims 41-44, wherein the peptide spacer comprises the hinge and CH₂CH₃ region of an IgG molecule.

46. The CAR of any one of claim 37-45, wherein the CAR is multispecific.

47. The CAR of claim 46, wherein the CAR is bispecific.

48. The CAR of claim 47, wherein the CAR comprises an antigen binding domain, wherein the antigen binding domain is an anti-CD3, anti-CD56, anti-CD8 antigen binding domain, and combinations thereof.

49. A composition comprising the antibody or antigen binding fragment of any one of claims 1-19, the polypeptide of any one of claims 20-36, or the CAR of any one of claims 37-48.

50. The composition of claim 49, wherein the composition comprises a pharmaceutical excipient.

51. The composition of claim 49 or 50, wherein the composition further comprises an adjuvant.

52. The composition of any one of claims 1-51, wherein the composition comprises at least two antibodies or antigen binding fragments.

53. One or more nucleic acids encoding the antibody or antigen binding fragment of any one of claims 1-19, the polypeptide of any one of claims 20-36, or the CAR of any one of claims 37-48.

54. A nucleic acid encoding an antibody heavy chain or VH, wherein the nucleic acid has at least 70% sequence identity to SEQ ID NO:21 or 23.

55. A nucleic acid encoding an antibody light chain or VL, wherein the nucleic acid has at least 70% sequence identity to SEQ ID NO:24 or 26.

56. A vector comprising the nucleic acid of any one of claims 53-55.

57. A cell comprising the antibody or antigen binding fragments of any one of claims 1-19, polypeptide of any one of claims 20-36, CAR of any one of claims 37-48, nucleic acid of any one of claims 53-55 or the vector of claim 56.

58. The cell of claim 57, wherein the cell is a human cell, B cell, T cell, Chinese hamster ovary, engineered cell, or immune cell.

59. The cell of claim 57 or 58, wherein the immune cell is a T cell or NK cell and the engineered cell is an engineered T cell or an engineered NK cell.

60. The cell of claim 59, wherein the T cell is a CD8⁺ T cell, CD4+ T cell, iNKT, or γδ T cell.

61. A method of a making a cell comprising transferring the nucleic acid(s) of any one of claims 53-55 or the vector of claim 56 into a cell.

62. The method of claim 61, wherein the method further comprises culturing the cell under conditions that allow for expression of a polypeptide from the nucleic acid.

63. The method of claim 62, wherein the method further comprising isolating the expressed polypeptide.

64. The method of any one of claims 61-63, wherein the cell is a human cell, B cell, T cell, Chinese hamster ovary, engineered cell, or immune cell.

65. The method of any one of claims 61-64, wherein the immune cell is a T cell or NK cell and the engineered cell is an engineered T cell or an NK cell.

66. The method of claim 65, wherein the T cell is a CD8⁺ T cell, CD4+ T cell, iNKT, or γδ T cell.

67. A method for making a polypeptide comprising transferring the nucleic acid(s) of any one of claims 53-55 or the vector of claim 56 into a cell under conditions sufficient to express polypeptides from the nucleic acids.

68. The method of claim 67, wherein the method further comprises isolating the expressed polypeptides.

69. A method for treating or preventing cancer in a subject, the method comprising administering to the subject, the antibody or antigen binding fragment of any one of claims 1-19, the polypeptide of any one of claims 20-36, the CAR of any one of claims 37-48, the composition of any one of claims 49-52, or the cell of any one of claims 57-60.

70. A method of stimulating an immune response in a subject, the method comprising administering the antibody or antigen binding fragment of any one of claims 1-19, the polypeptide of any one of claims 20-36, the CAR of any one of claims 37-48, the composition of any one of claims 49-52, or the cell of any one of claims 57-60.

71. A method for treating acute myelogenous leukemia in a subject, the method comprising administering to the subject an anti-B7-H3 antibody in combination with a BCL-2 inhibitor.

72. The method of claim 71, wherein the BCL-2 inhibitor comprises venetocolax.

73. The method of claim 71 or 72, wherein the anti-B7-H3 antibody comprises an antibody according to any one of claims 1-19.

74. The method of any one of claims 69-73, wherein the subject is a human subject.

75. The method of any one of claims 69-74, wherein the subject has been diagnosed with the cancer.

76. The method of any one of claims 69-75, wherein the cancer comprises a hematologic malignancy.

77. The method of claim 76, wherein the cancer comprises leukemia.

78. The method of claim 77, wherein the leukemia comprises acute myelogenous leukemia.

79. The method of any one of claims 69-75, wherein the cancer comprises breast cancer.

80. The method of claim 79, wherein the breast cancer comprises a B7-H3 expressing breast cancer.

81. The method of claim 79 or 80, wherein the breast cancer comprises a B7-H3^{high}-expressing breast cancer.

82. The method of any one of claims 79-82, wherein the breast cancer comprises basal, luminal A, luminal B, triple negative breast cancer, or Her2-enriched breast cancer.

83. The method of any one of claims 69-82, wherein the cancer comprises a B7-H3+-expressing cancer.

84. The method of any one of claims 69-83, wherein the cancer comprises a B7-H3^{high}-expressing cancer.

85. The method of any one of claims 69-84, wherein the subject has been determined to have high expression of B7-H3 in a biological sample from the subject compared to a control.

86. The method of claim 85, wherein the control comprises the level of expression of B7-H3 in non-cancerous cells.

87. The method of claim 85 or 86, wherein the biological sample from the subject comprises breast cancer cells and wherein the control comprises the level of expression of B7-H3 in non-cancerous mammary ducts.

88. The method of any one of claims 69-87, wherein cells are administered to the subject and wherein the cells are autologous.

89. The method of any one of claims 69-87, wherein cells are administered to the subject and wherein the cells are allogenic.

90. The method of any one of claims 69-89, wherein the subject has previously been treated for the cancer.

91. The method of claim 90, wherein the subject has been determined to be resistant to the previous treatment.

92. The method of any one of claims 69-91, wherein the method further comprises the administration of an additional therapy.

93. The method of claim 92, wherein the additional therapy comprises chemotherapy.

94. The method of claim 93, wherein the chemotherapy comprises cytarabine and/or daunorubicin.

95. The method of any one of claims 92-94, wherein the additional therapy comprises a targeted therapy.

96. The method of claim 95, wherein the targeted therapy comprises one or more of a FLT3 inhibitor, IDH inhibitor, BCL-2 inhibitor, or a hedgehog pathway inhibitor.

97. The method of claim 95 or 96, wherein the targeted therapy comprises one or more of midostaurin, gilteritinib, ivosidenib, enasidenib, gemtuzumab ozogamicin, venetoclax, and glasdegib.

98. The method of claim 97, wherein the targeted therapy comprises venetoclax.

99. The method of any eon of claims 92-98, wherein the combination of the antibody or antigen binding fragment, polypeptide, CAR, composition, or cell and the additional therapy enhances NK cell-mediated killing of cancer cells.

100. The method of any one of claims 69-99, wherein the cancer comprises stage I, II, III, or IV cancer.

101. The method of any one of claims 69-100, wherein the cancer comprises metastatic and/or recurrent cancer.

102. The method of any one of claims 69-101, wherein the cancer is a B7H3-positive cancer.

103. The method of any one of claims 69-102, wherein the subject has been determined to have B7H3-positive cancer cells.

104. A method for evaluating a sample from a subject, the method comprising contacting a biological sample from the subject, or extract thereof, with at least one antibody, antigen binding fragment, or polypeptide of any one of claims 1-36.

105. The method of claim 104, wherein the at least one antibody, antigen binding fragment, or polypeptide is operatively linked to a detectable label.

106. The method of claim 104 or 105, wherein the method further comprises incubating the antibody, antigen binding fragment, or polypeptide under conditions that allow for the binding of the antibody, antigen binding fragment, or polypeptide to antigens in the biological sample or extract thereof.

107. The method of any one of claims 104-106, wherein the method further comprises detecting the binding of an antigen to the antibody, antigen binding fragment, or polypeptide.

108. The method of any one of claims 104-107, wherein the method further comprises contacting the biological sample with at least one capture antibody, antigen, or polypeptide.

109. The method of claim 108, wherein the at least one capture antibody, antigen binding fragment, or polypeptide comprises at least one antibody, antigen binding fragment, or polypeptide of claims 1-36.

110. The method of claim 108 or 109, wherein the capture antibody is linked to a solid support.

111. The method of any one of claims 104-110, wherein the biological sample comprises a blood sample, tissue sample, or a sample obtained by a biopsy.

112. A method for treating cancer in a subject comprising administering to the subject an anticancer treatment after a biological sample from the subject has been analyzed for B7-H3 expression.

113. The method of claim 112, wherein the anticancer treatment comprises a B7-H3 targeting agent.

114. The method of claim 112 or 113, wherein the biological sample has been determined to have high expression of B7-H3.

115. The method of claim 114, wherein the biological sample has been determined to have high high expression of B7-H3 relative to a control, wherein the control is a cut-off value or wherein the control is level of B7-H3 expression in a biological sample from a subject or the average level of B7-H3 expression in biological samples from subjects determined to not have cancer.

116. The method of any one of claims 112-115, wherein the cancer comprises a hematologic malignancy.

117. The method of claim 116, wherein the cancer comprises leukemia.

118. The method of claim 117, wherein the leukemia comprises acute myelogenous leukemia.

119. The method of any one of claims 112-115, wherein the cancer comprises breast cancer.

120. The method of claim 119, wherein the breast cancer comprises a B7-H3 expressing breast cancer.

121. The method of any one of claims 119-120, wherein the breast cancer comprises basal, luminal A, luminal B, triple negative breast cancer, or Her2-enriched breast cancer.

122. The method of any one of claims 112-121, wherein the cancer comprises a B7-H3+-expressing cancer.

123. The method of any one of claims 112-122, wherein the cancer comprises a B7-H3^{high}-expressing cancer.

124. The method of any one of claims 112-123, wherein the cancer comprises a solid tumor.

125. The method of any one of claims 112-124, wherein the method further comprises administering at least one additional anticancer treatment.

126. The method of any one of claims 112-125, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

127. The method of any one of claims 112-126, wherein the biological sample comprises a tissue sample, a cancerous sample, a tumor sample, or a sample obtained from a biopsy.

128. A method for prognosing a subject having cancer or for predicting a cancer subject's response to an anticancer treatment, the method comprising evaluating a biological sample from the subject for B7-H3 expression.

129. The method of claim 128, wherein the anticancer treatment comprises a B7-H3 targeting agent.

130. The method of claim 128 or 129, wherein the biological sample has been evaluated as having high B7-H3 expression.

131. The method of claim 130, wherein the subject is prognosed as high risk for unfavorable clinical outcomes.

132. The method of claim 130 or 131, wherein the subject is predicted to respond an anticancer treatment comprising a B7-H3 targeting therapy.

133. The method of claim 128 or 129, wherein the biological sample has been evaluated as having low B7-H3 expression.

134. The method of claim 133, wherein the subject is prognosed as having favorable clinical outcomes.

135. The method of claim 133 or 134, wherein the subject is treated with an anticancer treatment and wherein the anticancer treatment excludes a B7-H3 targeting agent.

136. The method of any one of claims 131-135, wherein the clinical outcomes comprise one or more of overall survival, disease-free survival, progression-free survival, time-to-progression, and objective response rate.

137. The method of any one of claims 128-136, wherein the cancer comprises a hematologic malignancy.

138. The method of claim 137, wherein the cancer comprises leukemia.

139. The method of claim 138, wherein the leukemia comprises acute myelogenous leukemia.

140. The method of any one of claims 128-136, wherein the cancer comprises breast cancer.

141. The method of claim 140, wherein the breast cancer comprises a B7-H3 expressing breast cancer.

142. The method of any one of claims 140-141, wherein the breast cancer comprises basal, luminal A, luminal B, triple negative breast cancer, or Her2-enriched breast cancer.

143. The method of any one of claims 128-142, wherein the cancer comprises a B7-H3+-expressing cancer.

144. The method of any one of claims 128-143, wherein the cancer comprises a B7-H3^{high}-expressing cancer.

145. The method of any one of claims 128-144, wherein the cancer comprises a solid tumor.

146. The method of any one of claims 128-145, wherein the method further comprises administering at least one additional anticancer treatment.

147. The method of claim 146, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

148. The method of any one of claims 128-147, wherein the biological sample comprises a tissue sample, a cancerous sample, a tumor sample, or a sample obtained from a biopsy.

149. The method of any one of claims 113-148, wherein the B7-H3 targeting agent comprises an antibody, a polypeptide, an antigen binding fragment of an antibody, a single chain variable fragment (scFv) or a chimeric antigen receptor (CAR).

150. The method of any one of claims 113-149, wherein the B7-H3 targeting agent comprises a cell comprising a nucleic acid encoding an anti-B7H3 antibody, anti-B7H3 scFv, polypeptide, or CAR.

151. The method of claim 149 or 150, wherein the CAR comprises
a) an extracellular binding domain;
b) a transmembrane domain; and,
c) a cytoplasmic region comprising a costimulatory domain and a primary intracellular signaling domain.

152. The method of any one of claims 149-151, wherein the antibody, polypeptide, antigen binding fragment of an antibody, scFv, or extracellular binding comain of the CAR comprises a heavy chain variable region (VH) and a light chain variable region (VL), wherein the VH comprises a HCDR1, HCDR2, and HCDR3 from the VH of SEQ ID NO:3 and wherein the VL comprises a LCDR1, LCDR2, and LCDR3 from the VL of SEQ ID NO:13.

153. The method of any one of claims 149-152, wherein the antibody, polypeptide, antigen binding fragment of an antibody, scFv, or extracellular binding comain of the CAR comprises a VH and a VL, wherein the VH comprises a HCDR1, HCDR2, and HCDR3 having at least 80% sequence identity with a HCDR1, HCDR2, and HCDR3 from the VH of SEQ ID NO:3 and wherein the VL comprises a LCDR1, LCDR2, and LCDR3 having at least 80% sequence identity with a LCDR1, LCDR2, and LCDR3 from the VL of SEQ ID NO:13.

154. The method of any one of claims 149-153, wherein the antibody, polypeptide, antigen binding fragment of an antibody, scFv, or extracellular binding comain of the CAR comprises a VH and a VL, wherein the VH comprises a HCDR1, HCDR2, and HCDR3 comprising the amino acid sequence of SEQ ID NOS:4-6, respectively, and wherein the VL comprises a LCDR1, LCDR2, and LCDR3 comprising the amino acid sequence of SEQ ID NOS:14-16, respectively.

155. The method of any one of claims 149-154, wherein the antibody, polypeptide, antigen binding fragment of an antibody, scFv, or extracellular binding comain of the CAR comprises a VH and a VL, wherein the VH comprises a HCDR1, HCDR2, and HCDR3 having at least 80% sequence identity to SEQ ID NOS:4-6, respectively, and wherein the VL comprises a LCDR1, LCDR2, and LCDR3 having at least 80% sequence identity to SEQ ID NOS:14-16, respectively.

156. The method of any one of claims 149-155, wherein the VH comprises an amino acid sequence with at least 80% sequence identity to SEQ ID NO:3 and/or the VL comprises an amino acid sequence with at least 80% sequence identity to SEQ ID NO:13.

157. The method of any one of claims 149-156, wherein the antibody, polypeptide, antigen binding fragment of an antibody, scFv, or extracellular binding comain of the CAR comprises a heavy chain framework region 1-4 and light chain framework region 1-4 and wherein the heavy chain framework region 1-4 comprises an amino acid sequence with at least 80% sequence identity to SEQ ID NOS:7-10, respectively, and the light chain framework region 1-4 comprises an amino acid sequence with at least 80% sequence identity to SEQ ID NOS:17-20, respectively.

158. The method of claim 157, wherein the heavy chain framework region 1-4 comprises the amino acid sequence of SEQ ID NOS:7-10, respectively, and the light chain framework region 1-4 comprises the amino acid sequence of SEQ ID NOS:17-20, respectively.

159. The method of any one of claims 149-158, wherein the antibody, polypeptide, antigen binding fragment of an antibody, scFv, or extracellular binding comain of the CAR comprises an amino acid sequence with at least 70% sequence identity to one of SEQ ID NOS:1-20 and/or an amino acid sequence with 1 substitution relative to SEQ ID NOS:1-20.

160. The method of any one of claims 149-160, wherein the antibody, polypeptide, antigen binding fragment of an antibody, scFv, or extracellular binding comain of the CAR comprises a heavy chain and a light chain and wherein the heavy chain comprises an amino acid sequence with at least 70% sequence identity to SEQ ID NO:1 and the light chain comprises an amino acid sequence with at least 70% sequence identity to SEQ ID NO:11.

161. The method of any one of claims 149-160, wherein the antibody, polypeptide, antigen binding fragment of an antibody, scFv, or extracellular binding comain of the CAR comprises a signal peptide.

162. The method of claim 161, wherein the signal peptide comprises SEQ ID NO:2 or 12 or an amino acid sequence having at least 80% sequence identity to SEQ ID NO:2 or 12.

163. The method of any one of claims 149-162, wherein the B7-H3 targeting agent is an antibody and wherein the antibody is human, chimeric, or humanized.

164. The method of any one of claims 149-163, wherein the B7-H3 targeting agent is an antibody and wherein the antibody, or antigen binding fragment binds B7H3 with a K_{D} of about 10⁻⁶ nM to about 10⁻¹² pM.

165. The method of any one of claims 149-164, wherein the B7-H3 targeting agent is an antibody and wherein the antibody is a blocking antibody or a neutralizing antibody.

166. The method of any one of claims 149-162, wherein the B7-H3 targeting agent is a polypeptide, scFv, or CAR comprising an extracellular binding domain and wherein the VH is amino proximal to the VL.

167. The method of any one of claims 149-162, wherein the B7-H3 targeting agent is a polypeptide, scFv, or CAR comprising an extracellular binding domain and wherein the VL is amino proximal to the VH.

168. The method of any one of claims 149-167, wherein the B7-H3 targeting agent is a polypeptide, scFv, or CAR comprising an extracellular binding domain and wherein VH region and VL region are separated by a peptide linker.

169. The method of claim 168, wherein the peptide linker is a glycine-serine linker.

170. The method of claim 168 or 169, wherein the peptide linker is at least 4 amino acids.

171. The method of any one of claims 149-170, wherein the polypeptide, scFv, or CAR further comprises a second antigen binding region.

172. The method of claim 171, wherein the second antigen binding region specifically binds to CD3.

173. The method of any one of claims 149-171, wherein the polypeptide, scFv, or CAR further comprises a CD3 binding region.

174. The method of claim 173, wherein the CD3 binding region comprises a scFv that specifically binds to CD3.

175. The method of any one of claims 150-174, wherein the cell is a human cell, B cell, T cell, Chinese hamster ovary, engineered cell, or immune cell.

176. The method of claim 175, wherein the immune cell is a T cell or NK cell and the engineered cell is an engineered T cell or an engineered NK cell.

177. The method of claim 175, wherein the T cell is a CD8⁺ T cell, CD4+ T cell, iNKT, or γδ T cell.

178. A method comprising evaluating the expression level of B7-H3 in a biological sample from a subject having cancer.

179. The method of claim 178, wherein the biological sample has been evaluated as having high B7-H3 expression.

180. The method of claim 178, wherein the biological sample has been evaluated as having low B7-H3 expression.

181. The method of any one of claims 178-180, wherein the cancer comprises a hematologic malignancy.

182. The method of claim 181, wherein the cancer comprises leukemia.

183. The method of claim 182, wherein the leukemia comprises acute myelogenous leukemia.

184. The method of any one of claims 178-183, wherein the cancer comprises breast cancer.

185. The method of claim 184, wherein the breast cancer comprises a B7-H3 expressing breast cancer.

186. The method of any one of claims 184-185, wherein the breast cancer comprises basal, luminal A, luminal B, triple negative breast cancer, or Her2-enriched breast cancer.

187. The method of any one of claims 178-186, wherein the cancer comprises a B7-H3+-expressing cancer.

188. The method of any one of claims 178-187, wherein the cancer comprises a B7-3^{high}-expressing cancer.

189. The method of any one of claims 178-188, wherein the cancer comprises a solid tumor.

190. The method of any one of claims 178-189, wherein the biological sample comprises a tissue sample, a cancerous sample, a tumor sample, or a sample obtained from a biopsy.

191. The method of any one of claims 178-190, wherein the method further comprises comparing the level of expression to a control.

192. The method of claim 191, wherein the control is a cut-off value or wherein the control is level of B7-H3 expression in a biological sample from a subject or the average level of B7-H3 expression in biological samples from subjects determined to not have cancer.

193. An isolated peptide comprising at least 70% sequence identity to a peptide of SEQ ID NO:105 or 109.

194. The peptide of claim 193, wherein the peptide comprises at least 6 contiguous amino acids of a peptide of SEQ ID NO:105 or 109.

195. The peptide of claim 193 or 194, wherein the peptide is 13 amino acids or fewer in length.

196. The peptide of claim 195, wherein the peptide consists of 9 amino acids.

197. The peptide of any one of claims 193-196, wherein the peptide is immunogenic.

198. The peptide of any one of claims 193-197, wherein the peptide is modified.

199. The peptide of claim 198, wherein the modification comprises conjugation to a molecule.

200. The peptide of claim 198 or 199, wherein the molecule comprises an antibody, a lipid, an adjuvant, or a detection moiety.

201. The peptide of any of claims 193-200, wherein the peptide has at least 90% sequence identity to a peptide of SEQ ID NO:105 or 109.

202. The peptide of any of claims 193-201, wherein the peptide has 1, 2 or 3 substitutions relative to a peptide of SEQ ID NO:105 or 109.

203. The peptide of any one of claims 193-201, wherein the peptide comprises 100% sequence identity to a peptide of SEQ ID NO:105 or 109.

204. A pharmaceutical composition comprising the isolated peptide of any one of claims 193-203.

205. The pharmaceutical composition of claim 204, wherein the pharmaceutical composition is formulated for parenteral administration, intravenous injection, intramuscular injection, inhalation, or subcutaneous injection.

206. The pharmaceutical composition of claim 204 or 205, wherein the peptide is comprised in a liposome, lipid-containing nanoparticle, or in a lipid-based carrier.

207. The pharmaceutical composition of claim 206, wherein the pharmaceutical preparation is formulated for injection or inhalation as a nasal spray.

208. The pharmaceutical composition of any one of claims 204-207, wherein the composition is formulated as a vaccine.

209. The pharmaceutical composition of any one of claims 204-208, wherein the composition further comprises an adjuvant.

210. A nucleic acid encoding for the peptide of any one of claims 193-203.

211. An expression vector comprising the nucleic acid of claim 210.

212. A host cell comprising the nucleic acid of claim 210 or the expression vector of claim 211.

213. An in vitro isolated dendritic cell comprising the peptide of any one of claims 193-203, the nucleic acid of claim 210, or the expression vector of claim 211.

214. The dendritic cell of claim 213, wherein the dendritic cell is a mature dendritic cell.

215. A peptide-specific binding molecule, wherein the molecule specifically binds to a peptide of any one of claim 193-203.

216. The binding molecule of claim 215, wherein the binding molecule is an antibody, TCR mime antibody, scFv, camellid, aptamer, or DARPIN.

217. A method of making a cell comprising transferring the nucleic acid of claim 210 or the expression vector of claim 211 into the cell.

218. The method of claim 217, wherein the method further comprises isolating the expressed peptide or polypeptide.

219. A method of producing peptide-specific immune effector cells comprising:
(a) obtaining a starting population of immune effector cells; and
(b) contacting the starting population of immune effector cells with a peptide of any one of claims 193-203, thereby generating peptide-specific immune effector cells.

220. The method of claim 219, wherein contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface.

221. The method of claim 220, wherein the APCs are dendritic cells.

222. The method of any one of claims 219-221, wherein the immune effector cells are T cells, peripheral blood lymphocytes, NK cells, invariant NK cells, NKT cells.

223. The method of any one of claims 219-222, wherein the immune effector cells have been differentiated from mesenchymal stem cell (MSC) or induced pluripotent stem (iPS) cells.

224. The method of claim 222, wherein the T cells are CD8⁺ T cells, CD4⁺ T cells, or γδ T cells.

225. The method of claim 222, wherein the T cells are cytotoxic T lymphocytes (CTLs).

226. The method of any one of claims 219-225, wherein obtaining comprises isolating the starting population of immune effector cells from peripheral blood mononuclear cells (PBMCs).

227. The method of any one of claims 219-226, wherein the starting population of immune effector cells is obtained from a subject.

228. The method of claim 227, wherein the subject is a human.

229. The method of claim 227 or 228, wherein the subject has a cancer.

230. The method of claim 229, wherein the cancer comprises tumor cells that are positive for expression of the peptide.

231. The method of any one of claims 219-230, wherein the cancer comprises a hematologic malignancy.

232. The method of claim 231, wherein the cancer comprises leukemia.

233. The method of claim 232, wherein the leukemia comprises acute myelogenous leukemia.

234. The method of any one of claims 219-230, wherein the cancer comprises breast cancer.

235. The method of claim 234, wherein the breast cancer comprises a B7-H3 expressing breast cancer.

236. The method of any one of claims 234-235, wherein the breast cancer comprises basal, luminal A, luminal B, triple negative breast cancer, or Her2-enriched breast cancer.

237. The method of any one of claims 219-236, wherein the cancer comprises a B7-H3^{high}-expressing cancer.

238. The method of any one of claims 221-237, wherein the method further comprises introducing the peptide or a nucleic acid encoding the peptide into the dendritic cells prior to the co-culturing.

239. The method of claim 238, where the peptide or nucleic acids encoding the peptide are introduced by electroporation.

240. The method of claim 238, wherein the peptide or nucleic acids encoding the peptide are introduced by adding the peptide or nucleic acid encoding the peptide to the dendritic cell culture media.

241. The method of claim 238, wherein the immune effector cells are co-cultured with a second population of dendritic cells into which the peptide or the nucleic acid encoding the peptide has been introduced.

242. The method of claim 238, wherein a population of CD8 or CD4-positive and peptide MHC tetramer-positive T cells are purified from the immune effector cells following the co-culturing.

243. The method of claim 242, wherein a clonal population of peptide-specific immune effector cells are generated by limiting or serial dilution followed by expansion of individual clones by a rapid expansion protocol.

244. The method of claim 243, wherein the method further comprises cloning of a T cell receptor (TCR) from the clonal population of peptide-specific immune effector cells.

245. The method of claim 244, wherein cloning of the TCR is cloning of a TCR alpha and a beta chain.

246. The method of claim 244 or claim 245, wherein the TCR is cloned using a 5′-Rapid amplification of cDNA ends (RACE) method.

247. The method of claim 246, wherein the cloned TCR is subcloned into an expression vector.

248. The method of claim 247, wherein the expression vector is a retroviral or lentiviral vector.

249. The method of claim 248, where a host cell is transduced with the expression vector to generate an engineered cell that expresses the TCR.

250. The method of claim 249, wherein the host cell is an immune cell.

251. The method of any one of claims 221-250, wherein the immune cell is a NK cell or T cell and the engineered cell is an engineered NK cell or an engineered T cell.

252. The method of claim 251, wherein the T cell is a CD8⁺ T cell, CD4+ T cell, iNKT, or γδ T cell and the engineered cell is an engineered T cell.

253. The method of claim 252, wherein the starting population of immune effector cells is obtained from a subject with cancer and the host cell is allogeneic or autologous to the subject.

254. The method of claim 253, wherein the cancer is positive for expression of the peptide.

255. The method of claim 251 or 252, wherein a population of CD8 or CD4-positive and peptide MHC tetramer-positive engineered T cells are purified from the transduced host cells.

256. The method of claim 242, wherein a clonal population of peptide-specific engineered T cells are generated by limiting or serial dilution followed by expansion of individual clones by a rapid expansion protocol.

257. A peptide-specific engineered T cell produced according to any one of the methods of claims 219-230 or 249-256.

258. A pharmaceutical composition comprising the peptide-specific T cells produced according to any one of the methods of claims 219-230 or 249-256.

259. A method of treating or preventing cancer in a subject, the method comprising administering an effective amount of the peptide of any one of claims 193-203, the pharmaceutical composition of any one of claims 204-209 or 258, the nucleic acid or expression vector of claim 210 or 211, the dendritic cell of any one of claims 213-214, or the peptide-specific T cells of claim 257 to the subject.

260. A method of stimulating an immune response in a subject, the method comprising administering an effective amount of the peptide of any one of claims 193-203, the pharmaceutical composition of any one of claims 204-209 or 258, the nucleic acid or expression vector of claim 210 or 211, the dendritic cell of any one of claims 213-214, or the peptide-specific T cells of claim 257 to the subject.

261. The method of claim 259 or 260, wherein the subject is a human.

262. The method of any one of claims 259-261, wherein the peptide-specific T cells are autologous or allogeneic.

263. The method of any one of claims 259-262, further comprising administering at least a second therapeutic agent.

264. The method of claim 263, wherein the second therapeutic agent is an anticancer agent.

265. The method of any one of claims 259-264, wherein the subject has been diagnosed with cancer.

266. The method of claim 265, wherein the cancer comprises a cancer that is positive for expression of the peptide.

267. The method of any one of claims 259-266, wherein the cancer comprises a hematologic malignancy.

268. The method of claim 267, wherein the cancer comprises leukemia.

269. The method of claim 268, wherein the leukemia comprises acute myelogenous leukemia.

270. The method of any one of claims 259-266, wherein the cancer comprises breast cancer.

271. The method of claim 270, wherein the breast cancer comprises a B7-H3 expressing breast cancer.

272. The method of any one of claims 270-271, wherein the breast cancer comprises basal, luminal A, luminal B, triple negative breast cancer, or Her2-enriched breast cancer.

273. The method of any one of claims 259-272, wherein the cancer comprises a B7-H3^{high}-expressing cancer.

274. The method of any one of claims 259-273, wherein treating comprises one or more of reducing tumor size; increasing the overall survival rate; reducing the risk of recurrence of the cancer; reducing the risk of progression; and/or increasing the chance of progression-free survival, relapse-free survival, and/or recurrence-free survival.

275. A method of cloning a peptide-specific T cell receptor (TCR), the method comprising
(a) obtaining a starting population of immune effector cells;
(b) contacting the starting population of immune effector cells with the peptide of any one of claims 193-203, thereby generating peptide-specific immune effector cells;
(c) purifying immune effector cells specific to the peptide, and
(d) isolating a TCR sequence from the purified immune effector cells.

276. The method of claim 275, wherein contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface.

277. The method of claim 276, wherein the APCs are dendritic cells.

278. The method of claim 275, wherein the immune effector cells are T cells, peripheral blood lymphocytes, NK cells, invariant NK cells, NKT cells.

279. The method of claim 275, wherein the immune effector cells have been differentiated from mesenchymal stem cell (MSC) or induced pluripotent stem (iPS) cells.

280. The method of claim 278, wherein the T cells are CD8⁺ T cells, CD4⁺ T cells, or γδ T cells.

281. The method of claim 278, wherein the T cells are cytotoxic T lymphocytes (CTLs).

282. The method of any one of claims 275-282, wherein obtaining comprises isolating the starting population of immune effector cells from peripheral blood mononuclear cells (PBMCs).

283. The method of any of claims 275-282, wherein the starting population of immune effector cells is obtained from a subject.

284. The method of claim 283, wherein the subject is a human.

285. The method of claim 284, wherein the subject has cancer.

286. The method of claim 284, wherein the cancer comprises a hematologic malignancy or a solid tumor.

287. The method of any one of claims 277-286, wherein the method further comprises introducing the peptide or a nucleic acid encoding the peptide into the dendritic cells prior to the co-culturing.

288. The method of claim 287, where the peptide or nucleic acid encoding the peptide are introduced by electroporation.

289. The method of claim 287, wherein the peptide or nucleic acid encoding the peptide are introduced by adding the peptide or nucleic acid encoding the peptide to the media of the dendritic cells.

290. The method of claim 287, wherein the immune effector cells are co-cultured with a second population of dendritic cells into which the peptide or a nucleic acid encoding the peptide has been introduced.

291. The method of claim 287, wherein purifying is defined as purifying a population of CD4- or CD8-positive and peptide MHC tetramer-positive T cells from the immune effector cells following the co-culturing.

292. The method of claim 291, wherein the population of CD4- or CD8-positive and peptide MHC tetramer-positive T cells are purified by fluorescence activated cell sorting (FACS).

293. The method of claim 292, wherein purifying further comprises generation of a clonal population of peptide-specific immune effector cells by limiting or serial dilution of sorted cells followed by expansion of individual clones by a rapid expansion protocol.

294. The method of claim 293, wherein isolating is defined as cloning of a T cell receptor (TCR) from the clonal population of peptide-specific immune effector cells.

295. The method of any one of claims 275-294, wherein the method further comprises sequencing the TCR alpha and/or beta gene(s) and/or performing grouping of lymphocyte interactions by paratope hotspots (GLIPH) analysis.

296. The method of claim 294 or 295, wherein cloning of the TCR is cloning of a TCR alpha and a beta chain.

297. The method of claim 296, wherein the TCR alpha and beta chains are cloned using a 5′-Rapid amplification of cDNA ends (RACE) method.

298. The method of claim 297, wherein the cloned TCR is subcloned into an expression vector.

299. The method of claim 298, wherein the expression vector comprises a linker domain between the TCR alpha sequence and TCR beta sequence.

300. The method of claim 299, wherein the linker domain comprises a sequence encoding one or more peptide cleavage sites.

301. The method of claim 300, wherein the one or more cleavage sites are a Furin cleavage site and/or a P2A cleavage site.

302. The method of claim 301, wherein the TCR alpha sequence and TCR beta sequence are linked by an IRES sequence.

303. The method of any of claims 298-302, wherein the expression vector is a retroviral or lentiviral vector.

304. The method of claim 303, where a host cell is transduced with the expression vector to generate an engineered cell that expresses the TCR alpha and beta chains.

305. The method of claim 304, wherein the host cell is an immune cell.

306. A kit comprising the peptide of any one of claims 193-203 in a container.

307. The kit of claim 306, wherein the peptide is comprised in a pharmaceutical preparation.

308. The kit of claim 307, wherein the pharmaceutical preparation is formulated for parenteral administration or inhalation.
